SEQUENCE LISTING (58 sequences)

SEQ ID NO: 1 - RNA, 22 nt, Homo sapiens
aauugcacgguauccaucugua

SEQ ID NO: 2 - RNA, 22 nt, Homo sapiens
cacuagauugugagcuccugga

SEQ ID NO: 3 - RNA, 22 nt, Homo sapiens
ugagguaggagguuguauaguu

SEQ ID NO: 4 - RNA, 23 nt, Homo sapiens
ccuccguguuaccuguccucuag

SEQ ID NO: 5 - RNA, 22 nt, Homo sapiens
cuuucagucggauguuuacagc

SEQ ID NO: 6 - RNA, 22 nt, Homo sapiens
uuccugggcuucuccucuguag

SEQ ID NO: 7 - RNA, 22 nt, Homo sapiens
cuuucagucggauguuugcagc

SEQ ID NO: 8 - RNA, 22 nt, Homo sapiens
uguccucuagggccugcagucu

SEQ ID NO: 9 - RNA, 22 nt, Homo sapiens
cuauacgaccugcugccuuucu

SEQ ID NO: 10 - RNA, 22 nt, Homo sapiens
uuaucagaaucuccagggguac

SEQ ID NO: 11 - RNA, 22 nt, Homo sapiens
ucucugggccugugucuuaggc

SEQ ID NO: 12 - RNA, 22 nt, Homo sapiens
uuggcuggucucugcuccgcag

SEQ ID NO: 13 - RNA, 21 nt, Homo sapiens
ucacagugaaccggucucuuu

SEQ ID NO: 14 - RNA, 21 nt, Homo sapiens
cuccguuugccuguuucgcug

SEQ ID NO: 15 - RNA, 22 nt, Homo sapiens
uccgucucaguuacuuuauagc

SEQ ID NO: 16 - RNA, 21 nt, Homo sapiens
cuagacugaagcuccuugagg

SEQ ID NO: 17 - RNA, 22 nt, Homo sapiens
cugcccuagucuagcugaagcu

SEQ ID NO: 18 - RNA, 22 nt, Homo sapiens
uacccauugcauaucggaguug

SEQ ID NO: 19 - RNA, 22 nt, Homo sapiens
ugagguaguagauuguauaguu

SEQ ID NO: 20 - RNA, 22 nt, Homo sapiens
ugagguaguagguuguauaguu

SEQ ID NO: 21 - RNA, 23 nt, Homo sapiens
ucuacagugcacgugucuccagu

SEQ ID NO: 22 - RNA, 23 nt, Homo sapiens
agcagcauuguacagggcuauga

SEQ ID NO: 23 - RNA, 22 nt, Homo sapiens
uuucuauuucucaguggggcuc

SEQ ID NO: 24 - RNA, 22 nt, Homo sapiens
gcugacuccuaguccagggcuc

SEQ ID NO: 25 - DNA, 44 bp, Homo sapiens
ctcaactggtgtcgtggagtcggcaattcagttgagtacagatg

SEQ ID NO: 26 - DNA, 44 bp, Homo sapiens
ctcaactggtgtcgtggagtcggcaattcagttgagtccaggag

SEQ ID NO: 27 - DNA, 44 bp, Homo sapiens
ctcaactggtgtcgtggagtcggcaattcagttgagaactatac

SEQ ID NO: 28 - DNA, 44 bp, Homo sapiens
ctcaactggtgtcgtggagtcggcaattcagttgagctagagga

SEQ ID NO: 29 - DNA, 44 bp, Homo sapiens
ctcaactggtgtcgtggagtcggcaattcagttgaggctgtaaa

SEQ ID NO: 30 - DNA, 44 bp, Homo sapiens
ctcaactggtgtcgtggagtcggcaattcagttgagctacagag

SEQ ID NO: 31 - DNA, 44 bp, Homo sapiens
ctcaactggtgtcgtggagtcggcaattcagttgagagaaaggg

SEQ ID NO: 32 - DNA, 44 bp, Homo sapiens
ctcaactggtgtcgtggagtcggcaattcagttgaggtacccct

SEQ ID NO: 33 - DNA, 44 bp, Homo sapiens
ctcaactggtgtcgtggagtcggcaattcagttgagctgcggag

SEQ ID NO: 34 - DNA, 44 bp, Homo sapiens
ctcaactggtgtcgtggagtcggcaattcagttgagcagcgaaa

SEQ ID NO: 35 - DNA, 44 bp, Homo sapiens
ctcaactggtgtcgtggagtcggcaattcagttgaggctataaa

SEQ ID NO: 36 - DNA, 31 bp, Homo sapiens
acactccagctgggaattgcacggtatccat

SEQ ID NO: 37 - DNA, 31 bp, Homo sapiens
acactccagctgggcactagattgtgagctc

SEQ ID NO: 38 - DNA, 31 bp, Homo sapiens
acactccagctgggtgaggtaggaggttgta

SEQ ID NO: 39 - DNA, 31 bp, Homo sapiens
acactccagctgggcctccgtgttacctgtc

SEQ ID NO: 40 - DNA, 31 bp, Homo sapiens
acactccagctgggctttcagtcggatgttt

SEQ ID NO: 41 - DNA, 31 bp, Homo sapiens
acactccagctgggcttcctgggcttctcct

SEQ ID NO: 42 - DNA, 31 bp, Homo sapiens
acactccagctgggcctatacgacctgctgc

SEQ ID NO: 43 - DNA, 31 bp, Homo sapiens
acactccagctgggttatcagaatctccagg

SEQ ID NO: 44 - DNA, 31 bp, Homo sapiens
acactccagctgggcttggctggtctctgct

SEQ ID NO: 45 - DNA, 31 bp, Homo sapiens
acactccagctgggcctccgtttgcctgttt

SEQ ID NO: 46 - DNA, 31 bp, Homo sapiens
acactccagctgggctccgtctcagttactt

SEQ ID NO: 47 - DNA, 20 bp, Homo sapiens
ctcaactggtgtcgtggagt

SEQ ID NO: 48 - DNA, 18 bp, Homo sapiens
ttcagttgagtacagatg

SEQ ID NO: 49 - DNA, 18 bp, Homo sapiens
ttcagttgagtccaggag

SEQ ID NO: 50 - DNA, 18 bp, Homo sapiens
ttcagttgagaactatac

SEQ ID NO: 51 - DNA, 18 bp, Homo sapiens
ttcagttgagctagagga

SEQ ID NO: 52 - DNA, 18 bp, Homo sapiens
ttcagttgaggctgtaaa

SEQ ID NO: 53 - DNA, 18 bp, Homo sapiens
ttcagttgagctacagag

SEQ ID NO: 54 - DNA, 18 bp, Homo sapiens
ttcagttgagagaaaggg

SEQ ID NO: 55 - DNA, 18 bp, Homo sapiens
ttcagttgaggtacccct

SEQ ID NO: 56 - DNA, 18 bp, Homo sapiens
ttcagttgagctgcggag

SEQ ID NO: 57 - DNA, 18 bp, Homo sapiens
ttcagttgagcagcgaaa

SEQ ID NO: 58 - DNA, 18 bp, Homo sapiens
ttcagttgaggctataaa

(19) United States
(12) Patent Application Publication
(10) Pub. No.: US 2020/0308647 A1
(43) Pub. Date: Oct. 1, 2020

(54) MIRNAS AS BIOMARKERS FOR ALZHEIMER'S DISEASE

(71) Applicant: Hummingbird Diagnostics GMBH, Heidelberg (DE)

(72) Inventors: Andreas KELLER, Püttlingen (DE); Markus BEIER, Weinheim (DE)

(21) Appl. No.: 16/753,286

(22) PCT Filed: Oct. 23, 2018
(86) PCT No.: PCT/EP2018/079020

§ 371 Date: Apr. 2, 2020

(30) Foreign Application Priority Data
Oct. 25, 2017 (EP) 17198253.1

(51) Int. Cl. C12Q 1/6883

(52) U.S. Cl. CPC C12Q 1/6883; G01N 2800/2821; C12Q 2600/178; C12Q 2600/158

(57) ABSTRACT

The present invention relates to methods for determining Alzheimer's Disease (AD) in a patient. Further, the present invention relates to uses of polynucleotides for detecting mi RNAs in a blood sample isolated from a patient for determining Alzheimer's Disease in the patient. Furthermore, the invention relates to kits for determining Alzheimer's Disease in a patient.

### CROSS REFERENCE TO RELATED APPLICATIONS

This application is a U.S. National Stage of PCT/EP2018/079020, International Filing Date Oct. 23, 2018 which claims priority to European Patent Application No. 17198253.1, filing date Oct. 25, 2017, each of which is incorporated herein by reference.

### REFERENCE TO SUBMISSION OF A SEQUENCE LISTING AS A TEXT FILE

The Sequence Listing written in file 095697-1187528_Sequence_Listing.txt created on Apr. 1, 2020, 8.68 bytes, machine format IBM-PC, MS-Windows operating system, is hereby incorporated by reference in its entirety for all purposes.

The present invention relates to methods for determining Alzheimer's Disease (AD) in a patient. Further, the present invention relates to uses of polynucleotides for detecting miRNAs in a blood sample isolated from a patient for determining Alzheimer's Disease in the patient. Furthermore, the invention relates to kits for determining Alzheimer's Disease in a patient.

### BACKGROUND OF THE INVENTION

Molecular diagnostics has increasingly gained in importance. It has found an entry into the clinical diagnosis of diseases (inter alia detection of infectious pathogens, detection of mutations of the genome, detection of diseased cells and identification of risk factors for predisposition to a disease). In particular, through the determination of gene expression in biological samples such as bodily fluids and tissues, nucleic acid analysis opens up very promising new possibilities in the study and diagnosis of diseases.

Nucleic acids of interest to be detected include genomic DNA, expressed mRNA and other RNAs such as microRNAs (abbreviated miRNAs). MiRNAs are a new class of small RNAs with various biological functions. They are short (average of 20-24 nucleotide) ribonucleic acid (RNA) molecules found in eukaryotic cells. Several hundred different species of miRNAs (i.e. several hundred different sequences) have been identified in mammals. They are important for post-transcriptional gene-regulation and bind to complementary sequences on target messenger RNA transcripts (mRNAs), which can lead to translational repression or target degradation and gene silencing. As such they can also be used as biologic markers for research, diagnosis, and therapy purposes.

Alzheimer's Disease (AD), also known in medical literature as Alzheimer disease, is the most common form of dementia. Alzheimer's Disease is characterized by loss of neurons and synapses in the cerebral cortex and certain subcortical regions and leads to a gross degeneration in these regions. In AD protein misfolding and aggregation (formation of so-called “plaques”) in the brain is caused by accumulation of abnormally folded A-beta and tau proteins in the affected tissues.

Early symptoms of AD are often mistaken to be age-related problems. In early stages of AD, the most common symptom is difficulty in remembering recent events. When AD is suspected, the diagnosis is usually confirmed with functional tests that evaluate behavior and cognitive abilities, often followed by imaging analysis of the brain. Imaging methods used for this purpose include computed tomography (CT), magnetic resonance imaging (MRI), single photon emission computed tomography (SPECT), and positron emission tomography (PET). However, examination of the brain tissue (post mortem) is needed for a definite diagnosis. In patients already having dementia, SPECT appears to be superior in differentiating Alzheimer's Disease from other possible causes, compared with the usual attempts employing mental testing and medical history analysis. Nevertheless, the above described diagnosis of AD is time consuming, expensive, and difficult.

MiRNAs have already been suggested as non-invasive biomarkers for the diagnosis of Alzheimer's Disease. In particular, the detection of miRNAs in a biological sample of a patient in order to determine whether the patient suffers from Alzheimer's Disease has already been recommended. However, the diagnostic power of the described miRNA based diagnostics tests and the reliability of the data obtained therefrom leaves much to be desired. In particular, the reliable and early diagnosis of AD based on non-invasive molecular biomarkers remains a challenge. In particular, the miRNA signatures presently suggested only allow the analysis of a specific group of people (regionally limited, country-specific).

Therefore, there exists an unmet need for a powerful and reliable diagnostic test for AD. This test should allow to measure region-unspecific, in particular cohorts from different countries. Another clinical need is to guide the therapy and to monitor the disease status of patients.

The present invention meets these needs. Especially, the present inventors found new Alzheimer signatures that hold in different countries and for groups with different ethnical background.

### SUMMARY OF THE INVENTION

In a first aspect, the present invention relates to a method for determining Alzheimer's Disease in a patient comprising the steps of:
(i) determining the level (of each) of at least 3 miRNAs comprised in a set in a blood sample isolated from a patient, wherein the first miRNA has a nucleotide sequence according to SEQ ID NO: 1 (hsa-miR-363-3p), and
(ii) comparing the level (of each) of the at least 3 miRNAs comprised in the set with a reference level of said at least three miRNAs, wherein the comparison allows to determine Alzheimer's Disease in the patient.

In a second aspect, the present invention relates to a method for determining Alzheimer's Disease in a patient comprising the steps of:
(i) determining the level (of each) of at least 3 miRNAs in a blood sample isolated from a patient, wherein the at least three miRNAs are comprised in a set selected from the group consisting of:
   (a) SEQ ID NO: 1, SEQ ID NO: 2, and SEQ ID NO: 9,
   (b) SEQ ID NO: 1, SEQ ID NO: 2, and SEQ ID NO: 4,
   (c) SEQ ID NO: 1, SEQ ID NO: 2, and SEQ ID NO: 5,
   (d) SEQ ID NO: 1, SEQ ID NO: 2, and SEQ ID NO: 6,
   (e) SEQ ID NO: 1, SEQ ID NO: 2, SEQ ID NO: 6, and SEQ ID NO: 9,
   (f) SEQ ID NO: 1, SEQ ID NO: 2, SEQ ID NO: 5, and SEQ ID NO: 12,
   (g) SEQ ID NO: 1, SEQ ID NO: 2, SEQ ID NO: 3, SEQ ID NO: 6, SEQ ID NO: 8, and SEQ ID NO: 10,
   (h) SEQ ID NO: 1, SEQ ID NO: 2, SEQ ID NO: 3, SEQ ID NO: 6, and SEQ ID NO: 8,
   (i) SEQ ID NO: 1, SEQ ID NO: 2, SEQ ID NO: 5, SEQ ID NO: 10, SEQ ID NO: 12, and SEQ ID NO: 13,
   (j) SEQ ID NO: 1, SEQ ID NO: 2, SEQ ID NO: 3, SEQ ID NO: 5, SEQ ID NO: 6, and SEQ ID NO: 11,
   (k) SEQ ID NO: 1, SEQ ID NO: 2, SEQ ID NO: 3, SEQ ID NO: 4, SEQ ID NO: 7, SEQ ID NO: 8, and SEQ ID NO: 11,
   (l) SEQ ID NO: 1, SEQ ID NO: 2, SEQ ID NO: 12, and SEQ ID NO: 13,
   (m) SEQ ID NO: 1, SEQ ID NO: 2, SEQ ID NO: 5, SEQ ID NO: 9, and SEQ ID NO: 12,
   (n) SEQ ID NO: 1, SEQ ID NO: 2, SEQ ID NO: 7, SEQ ID NO: 9, and SEQ ID NO: 12,
   (o) SEQ ID NO: 1, SEQ ID NO: 2, SEQ ID NO: 9, SEQ ID NO: 10, and SEQ ID NO: 12,
   (p) SEQ ID NO: 1, SEQ ID NO: 2, and SEQ ID NO: 12,
   (q) SEQ ID NO: 1, SEQ ID NO: 2, and SEQ ID NO: 14,
   (r) SEQ ID NO: 1, SEQ ID NO: 2, SEQ ID NO: 9, and SEQ ID NO: 12,
   (s) SEQ ID NO: 1, SEQ ID NO: 2, and SEQ ID NO: 15,
   (t) SEQ ID NO: 1, SEQ ID NO: 3, SEQ ID NO: 4, SEQ ID NO: 7, and SEQ ID NO: 11,
   (u) SEQ ID NO: 1, SEQ ID NO: 3, SEQ ID NO: 5, SEQ ID NO: 8, SEQ ID NO: 9, and SEQ ID NO: 10,
   (v) SEQ ID NO: 1, SEQ ID NO: 3, SEQ ID NO: 4, SEQ ID NO: 5, SEQ ID NO: 7, SEQ ID NO: 11, and SEQ ID NO: 16,
   (w) SEQ ID NO: 1, SEQ ID NO: 3, SEQ ID NO: 5, SEQ ID NO: 6, SEQ ID NO: 8, SEQ ID NO: 16, and SEQ ID NO: 17,
   (x) SEQ ID NO: 1, SEQ ID NO: 3, SEQ ID NO: 5, SEQ ID NO: 8, SEQ ID NO: 16, SEQ ID NO: 17, and SEQ ID NO: 18, and
   (y) SEQ ID NO: 1, SEQ ID NO: 3, SEQ ID NO: 6, SEQ ID NO: 7, SEQ ID NO: 8, SEQ ID NO: 16, and SEQ ID NO: 17,
   and
(ii) comparing the level (of each) of the at least 3 miRNAs comprised in the set with a reference level of said at least three miRNAs, wherein the comparison allows to determine Alzheimer's Disease in the patient.

In a third aspect, the present invention relates to the use of at least three polynucleotides for detecting at least three miRNAs comprised in a set in a blood sample isolated from a patient for determining Alzheimer's Disease in the patient, wherein the first miRNA has a nucleotide sequence according to SEQ ID NO: 1 (hsa-miR-363-3p).

In a fourth aspect, the present invention relates to the use of at least three polynucleotides for detecting at least three miRNAs in a blood sample isolated from a patient for determining Alzheimer's Disease in the patient, wherein the at least three miRNAs are comprised in a set selected from the group consisting of:
(a) SEQ ID NO: 1, SEQ ID NO: 2, and SEQ ID NO: 9,
(b) SEQ ID NO: 1, SEQ ID NO: 2, and SEQ ID NO: 4,
(c) SEQ ID NO: 1, SEQ ID NO: 2, and SEQ ID NO: 5,
(d) SEQ ID NO: 1, SEQ ID NO: 2, and SEQ ID NO: 6,
(e) SEQ ID NO: 1, SEQ ID NO: 2, SEQ ID NO: 6, and SEQ ID NO: 9,
(f) SEQ ID NO: 1, SEQ ID NO: 2, SEQ ID NO: 5, and SEQ ID NO: 12,
(g) SEQ ID NO: 1, SEQ ID NO: 2, SEQ ID NO: 3, SEQ ID NO: 6, SEQ ID NO: 8, and SEQ ID NO: 10,
(h) SEQ ID NO: 1, SEQ ID NO: 2, SEQ ID NO: 3, SEQ ID NO: 6, and SEQ ID NO: 8,
(i) SEQ ID NO: 1, SEQ ID NO: 2, SEQ ID NO: 5, SEQ ID NO: 10, SEQ ID NO: 12, and SEQ ID NO: 13,
(j) SEQ ID NO: 1, SEQ ID NO: 2, SEQ ID NO: 3, SEQ ID NO: 5, SEQ ID NO: 6, and SEQ ID NO: 11,
(k) SEQ ID NO: 1, SEQ ID NO: 2, SEQ ID NO: 3, SEQ ID NO: 4, SEQ ID NO: 7, SEQ ID NO: 8, and SEQ ID NO: 11,
(l) SEQ ID NO: 1, SEQ ID NO: 2, SEQ ID NO: 12, and SEQ ID NO: 13,
(m) SEQ ID NO: 1, SEQ ID NO: 2, SEQ ID NO: 5, SEQ ID NO: 9, and SEQ ID NO: 12,
(n) SEQ ID NO: 1, SEQ ID NO: 2, SEQ ID NO: 7, SEQ ID NO: 9, and SEQ ID NO: 12,
(o) SEQ ID NO: 1, SEQ ID NO: 2, SEQ ID NO: 9, SEQ ID NO: 10, and SEQ ID NO: 12,
(p) SEQ ID NO: 1, SEQ ID NO: 2, and SEQ ID NO: 12,
(q) SEQ ID NO: 1, SEQ ID NO: 2, and SEQ ID NO: 14,
(r) SEQ ID NO: 1, SEQ ID NO: 2, SEQ ID NO: 9, and SEQ ID NO: 12,
(s) SEQ ID NO: 1, SEQ ID NO: 2, and SEQ ID NO: 15,
(t) SEQ ID NO: 1, SEQ ID NO: 3, SEQ ID NO: 4, SEQ ID NO: 7, and SEQ ID NO: 11,
(u) SEQ ID NO: 1, SEQ ID NO: 3, SEQ ID NO: 5, SEQ ID NO: 8, SEQ ID NO: 9, and SEQ ID NO: 10,
(v) SEQ ID NO: 1, SEQ ID NO: 3, SEQ ID NO: 4, SEQ ID NO: 5, SEQ ID NO: 7, SEQ ID NO: 11, and SEQ ID NO: 16,
(w) SEQ ID NO: 1, SEQ ID NO: 3, SEQ ID NO: 5, SEQ ID NO: 6, SEQ ID NO: 8, SEQ ID NO: 16, and SEQ ID NO: 17,
(x) SEQ ID NO: 1, SEQ ID NO: 3, SEQ ID NO: 5, SEQ ID NO: 8, SEQ ID NO: 16, SEQ ID NO: 17, and SEQ ID NO: 18, and
(y) SEQ ID NO: 1, SEQ ID NO: 3, SEQ ID NO: 6, SEQ ID NO: 7, SEQ ID NO: 8, SEQ ID NO: 16, and SEQ ID NO: 17.

In a fifth aspect, the present invention relates to a kit for determining Alzheimer's Disease in a patient comprising:
(i) means for determining the level (of each) of at least three miRNAs comprised in a set in a blood sample isolated from a patient, wherein the first miRNA has a nucleotide sequence according to SEQ ID NO: 1 (hsa-miR-363-3p), and
(ii) optionally at least three references.

In a sixth aspect, the present invention relates to a kit for determining Alzheimer's Disease in a patient comprising:
(i) means for determining the level (of each) of at least three miRNAs in a blood sample isolated from a patient, wherein the at least three miRNAs are comprised in a set selected from the group consisting of:
   (a) SEQ ID NO: 1, SEQ ID NO: 2, and SEQ ID NO: 9,
   (b) SEQ ID NO: 1, SEQ ID NO: 2, and SEQ ID NO: 4,
   (c) SEQ ID NO: 1, SEQ ID NO: 2, and SEQ ID NO: 5,
   (d) SEQ ID NO: 1, SEQ ID NO: 2, and SEQ ID NO: 6,
   (e) SEQ ID NO: 1, SEQ ID NO: 2, SEQ ID NO: 6, and SEQ ID NO: 9,
   (f) SEQ ID NO: 1, SEQ ID NO: 2, SEQ ID NO: 5, and SEQ ID NO: 12,
   (g) SEQ ID NO: 1, SEQ ID NO: 2, SEQ ID NO: 3, SEQ ID NO: 6, SEQ ID NO: 8, and SEQ ID NO: 10,
   (h) SEQ ID NO: 1, SEQ ID NO: 2, SEQ ID NO: 3, SEQ ID NO: 6, and SEQ ID NO: 8,
   (i) SEQ ID NO: 1, SEQ ID NO: 2, SEQ ID NO: 5, SEQ ID NO: 10, SEQ ID NO: 12, and SEQ ID NO: 13,
   (j) SEQ ID NO: 1, SEQ ID NO: 2, SEQ ID NO: 3, SEQ ID NO: 5, SEQ ID NO: 6, and SEQ ID NO: 11,
   (k) SEQ ID NO: 1, SEQ ID NO: 2, SEQ ID NO: 3, SEQ ID NO: 4, SEQ ID NO: 7, SEQ ID NO: 8, and SEQ ID NO: 11,
   (l) SEQ ID NO: 1, SEQ ID NO: 2, SEQ ID NO: 12, and SEQ ID NO: 13,
   (m) SEQ ID NO: 1, SEQ ID NO: 2, SEQ ID NO: 5, SEQ ID NO: 9, and SEQ ID NO: 12,
   (n) SEQ ID NO: 1, SEQ ID NO: 2, SEQ ID NO: 7, SEQ ID NO: 9, and SEQ ID NO: 12,
   (o) SEQ ID NO: 1, SEQ ID NO: 2, SEQ ID NO: 9, SEQ ID NO: 10, and SEQ ID NO: 12,
   (p) SEQ ID NO: 1, SEQ ID NO: 2, and SEQ ID NO: 12,
   (q) SEQ ID NO: 1, SEQ ID NO: 2, and SEQ ID NO: 14,
   (r) SEQ ID NO: 1, SEQ ID NO: 2, SEQ ID NO: 9, and SEQ ID NO: 12,
   (s) SEQ ID NO: 1, SEQ ID NO: 2, and SEQ ID NO: 15,
   (t) SEQ ID NO: 1, SEQ ID NO: 3, SEQ ID NO: 4, SEQ ID NO: 7, and SEQ ID NO: 11,
   (u) SEQ ID NO: 1, SEQ ID NO: 3, SEQ ID NO: 5, SEQ ID NO: 8, SEQ ID NO: 9, and SEQ ID NO: 10,
   (v) SEQ ID NO: 1, SEQ ID NO: 3, SEQ ID NO: 4, SEQ ID NO: 5, SEQ ID NO: 7, SEQ ID NO: 11, and SEQ ID NO: 16,
   (w) SEQ ID NO: 1, SEQ ID NO: 3, SEQ ID NO: 5, SEQ ID NO: 6, SEQ ID NO: 8, SEQ ID NO: 16, and SEQ ID NO: 17,
   (x) SEQ ID NO: 1, SEQ ID NO: 3, SEQ ID NO: 5, SEQ ID NO: 8, SEQ ID NO: 16, SEQ ID NO: 17, and SEQ ID NO: 18, and
   (y) SEQ ID NO: 1, SEQ ID NO: 3, SEQ ID NO: 6, SEQ ID NO: 7, SEQ ID NO: 8, SEQ ID NO: 16, and SEQ ID NO: 17,
   and
(ii) optionally at least three references.

This summary of the invention does not necessarily describe all features and/or all aspects of the present invention. Other embodiments will become apparent from a review of the ensuing detailed description.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention. In the event of a conflict between the definitions or teachings of such incorporated references and definitions or teachings recited in the present specification, the text of the present specification takes precedence.

The term “comprise” or variations such as “comprises” or “comprising” according to the present invention means the inclusion of a stated integer or group of integers but not the exclusion of any other integer or group of integers. The term “consisting essentially of” according to the present invention means the inclusion of a stated integer or group of integers, while excluding modifications or other integers which would materially affect or alter the stated integer. The term “consisting of” or variations such as “consists of” according to the present invention means the inclusion of a stated integer or group of integers and the exclusion of any other integer or group of integers.

The terms “a” and “an” and “the” and similar reference used in the context of describing the invention (especially in the context of the claims) are to be construed to cover both the singular and the plural, unless otherwise indicated herein or clearly contradicted by context.

The terms “microRNA” or “miRNA”, as used herein, refer to single-stranded RNA molecules of at least 10 nucleotides and of not more than 45 nucleotides covalently linked together. Preferably, the polynucleotides used in the present invention are molecules of 10 to 45 nucleotides or 15 to 35 nucleotides in length, more preferably of 16 to 28 nucleotides or 17 to 27 nucleotides in length, i.e. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides in length, not including optionally labels and/or elongated sequences (e.g. biotin stretches). The miRNAs regulate gene expression and are encoded by genes from whose DNA they are transcribed but miRNAs are not translated into protein (i.e. miRNAs are non-coding RNAs). The genes encoding miRNAs are longer than the processed mature miRNA molecules. The miRNAs are first transcribed as primary transcripts or pri-miRNAs with a cap and poly-A tail and processed to short, 70 nucleotide stem-loop structures known as pre-miRNAs in the cell nucleus. This processing is performed in animals by a protein complex known as the Microprocessor complex consisting of the nuclease Drosha and the double-stranded RNA binding protein Pasha. These pre-miRNAs are then processed to mature miRNAs in the cytoplasm by interaction with the endonuclease Dicer, which also initiates the formation of the RNA-induced silencing complex (RISC). When Dicer cleaves the pre-miRNA stem-loop, two complementary short RNA molecules are formed, but only one is integrated into the RISC. This strand is known as the guide strand and is selected by the argonaute protein, the catalytically active RNase in the RISC, on the basis of the stability of the 5′ end. The remaining strand, known as the miRNA*, anti-guide (anti-strand), or passenger strand, is degraded as a RISC substrate. Therefore, the miRNA*s are derived from the same hairpin structure like the “normal” miRNAs. So if the “normal” miRNA is then later called the “mature miRNA” or “guide strand”, the miRNA* is the “anti-guide strand” or “passenger strand”. This processing is referred to be the canonical miRNA processing pathway.

The terms “microRNA*” or “miRNA*”, as used herein, refer to single-stranded RNA molecules of at least 10 nucleotides and of not more than 35 nucleotides covalently linked together. Preferably, the polynucleotides used in the present invention are molecules of 10 to 45 nucleotides or 15 to 35 nucleotides in length, more preferably of 16 to 28 nucleotides or 18 to 23 nucleotides in length, i.e. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides in length, not including optionally labels and/or elongated sequences (e.g. biotin stretches). The “miRNA*s”, also known as the “anti-guide strands” or “passenger strands”, are mostly complementary to the “mature miRNAs” or “guide strands”, but have usually single-stranded overhangs on each end. There are usually one or more mispairs and there are sometimes extra or missing bases causing single-stranded “bubbles”. The miRNA*s are likely to act in a regulatory fashion as the miRNAs (see also above). In the context of the present invention, the terms “miRNA” and “miRNA*” are interchangeable used.

The term “miRBase”, as used herein, refers to a well-established repository of validated miRNAs. The miRBase is a searchable database of published miRNA sequences and annotation. Each entry in the miRBase Sequence database represents a predicted hairpin portion of a miRNA transcript (termed mir in the database), with information on the location and sequence of the mature miRNA sequence (termed miR). Both hairpin and mature sequences are available for searching and browsing, and entries can also be retrieved by name, keyword, references and annotation. All sequence and annotation data are also available for download.

The miRNAs described herein have, comprise, essentially consist of, or consist of a nucleotide sequence selected from the group consisting of SEQ II) NO: 1 to SEQ II) NO: 24.

The term “nucleotides”, as used herein, refers to structural components, or building blocks, of DNA and RNA. Nucleotides consist of a base (one of four chemicals: adenine, thymine, guanine, and cytosine) plus a molecule of sugar and one of phosphoric acid. The term “nucleosides” refers to glycosylamine consisting of a nucleobase (often referred to simply base) bound to a ribose or deoxyribose sugar. Examples of nucleosides include cytidine, uridine, adenosine, guanosine, thymidine and inosine. Nucleosides can be phosphorylated by specific kinases in the cell on the sugar's primary alcohol group (—CH2-OH), producing nucleotides, which are the molecular building blocks of DNA and RNA.

The term “polynucleotide”, as used herein, means a molecule of at least 10 nucleotides and of not more than 35 nucleotides covalently linked together. Preferably, the polynucleotides used in the present invention are molecules of 10 to 35 nucleotides or 15 to 45 nucleotides in length, more preferably of 16 to 28 nucleotides or 17 to 27 nucleotides in length, i.e. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 nucleotides in length, not including optionally spacer elements and/or elongation elements described below. The depiction of a single strand of a polynucleotide also defines the sequence of the complementary strand. Polynucleotides may be single stranded or double stranded, or may contain portions of both double stranded and single stranded sequences. The term “polynucleotide” means a polymer of deoxyribonucleotide or ribonucleotide bases and includes DNA and RNA molecules, both sense and anti-sense strands. In detail, the polynucleotide may be DNA, both cDNA and genomic DNA, RNA, cRNA or a hybrid, where the polynucleotide sequence may contain combinations of deoxyribonucleotide or ribonucleotide bases, and combinations of bases including uracil, adenine, thymine, cytosine, guanine, inosine, xanthine, hypoxanthine, isocytosine and isoguanine. Polynucleotides may be obtained by chemical synthesis methods or by recombinant methods.

In the context of the present invention, a polynucleotide as a single polynucleotide strand provides a probe (e.g. miRNA capture probe) that is capable of binding to, hybridizing with, or detecting a target of complementary sequence, such as a nucleotide sequence of a miRNA or miRNA*, through one or more types of chemical bonds, usually through complementary base pairing, usually through hydrogen bond formation. Polynucleotides in their function as probes may bind target sequences, such as nucleotide sequences of miRNAs or miRNAs*, lacking complete complementarity with the polynucleotide sequences depending upon the stringency of the hybridization condition. There may be any number of base pair mismatches which will interfere with hybridization between the target sequence, such as a nucleotide sequence of a miRNA or miRNA*, and the single stranded polynucleotide described herein. However, if the number of mutations is so great that no hybridization can occur under even the least stringent hybridization conditions, the sequences are no complementary sequences. The polynucleotide variants including polynucleotide fragments or polynucleotide mutants and the miRNA variants including miRNA fragments or miRNA mutants are further defined below. Described herein are polynucleotides in form of single polynucleotide strands as probes for binding to, hybridizing with or detecting complementary sequences of miRNAs (targets), which are selected from the group consisting of SEQ ID NO: 1 to SEQ ID NO: 24.

The polynucleotide, e.g. the polynucleotide used as a probe for detecting a miRNA or miRNA*, may be unlabeled, directly labeled, or indirectly labeled, such as with biotin to which a streptavidin complex may later bind. The polynucleotide, e.g. the polynucleotide used as a probe for detecting a miRNA or miRNA*, may also be modified, e.g. may comprise an elongation (EL) element. For use in a RAKE or MPEA assay, a polynucleotide with an elongation element may be used as a probe. The elongation element comprises a nucleotide sequence with 1 to 30 nucleotides chosen on the basis of showing low complementarity to potential target sequences, such as nucleotide sequences of miRNAs or miRNAs*, therefore resulting in not to low degree of cross-hybridization to a target mixture. Preferred is a homomeric sequence stretch Nₙ with n=1 to 30, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, and N=A or C, or T or G. Particularly preferred is a homomeric sequence stretch Nₙ with n=1 to 12, i.e. 1, 2, 3, 4, 5, 6, 7, 8, 9, 10, 11, or 12, and N=A or C, or T or G. The polynucleotide, e.g. the polynucleotide used as a probe for detecting a miRNA or miRNA*, may be present in form of a tandem, i.e. in form of a polynucleotide hybrid of two different or identical polynucleotides, both in the same orientation, i.e. 5′ to 3′ or 3′ to 5′, or in different orientation, i.e. 5′ to 3′ and 3′ to 5′. Said polynucleotide hybrid/tandem may comprise a spacer element. For use in a tandem hybridization assay, the polynucleotide hybrid/tandem as a probe may comprise a spacer (SP) element. The spacer element represents a nucleotide sequence with n=0 to 12, i.e. 0, 1, 2, 3, 4, 5, 6, 7, 8, 9, 10, 11, or 12, nucleotides chosen on the basis of showing low complementarity to potential target sequences, such as nucleotide sequences of miRNAs or anti-miRNAs, therefore resulting in not to low degree of cross-hybridization to a target mixture. It is preferred that n is 0, i.e. that there is no spacer between the two miRNA sequence stretches.

The term “differential expression” of a nucleic acid molecule, as used herein, refers to a qualitative and/or quantitative difference in the temporal and/or local nucleic acid molecule expression pattern, e.g. within and/or among blood samples. Thus, a differentially expressed nucleic acid molecule may qualitatively have its expression altered, including an activation or inactivation in, for example, blood from a diseases subject versus blood from a healthy subject. The difference in nucleic acid molecule expression may also be quantitative, e.g. in that expression is modulated, i.e. either up-regulated, resulting in an increased amount of the nucleic acid molecule, or down-regulated, resulting in a decreased amount of the nucleic acid molecule. The degree to which nucleic acid molecule expression differs need only be large enough to be quantified via standard expression characterization techniques, e.g. by quantitative hybridization (e.g. to a microarray, to beads), amplification (PCR, RT-PCR, qRT-PCR, high-throughput RT-PCR), ELISA for quantitation, next generation sequencing (e.g. ABI SOLID, Illumina Genome Analyzer, Roche 454 GS FL, BGISEQ), flow cytometry (e.g. LUMINEX) and the like.

The term “label”, as used herein, means a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and other entities which can be made detectable. A label may be incorporated into nucleic acids at any position, e.g. at the 3′ or 5′ end or internally. The polynucleotide for detecting a miRNA (polynucleotide probe) and/or the miRNA itself may be labeled.

The term “stringent hybridization conditions”, as used herein, means conditions under which a first nucleic acid sequence (e.g. polynucleotide in its function as a probe for detecting a miRNA or miRNA*) will hybridize to a second nucleic acid sequence (e.g. target sequence such as nucleotide sequence of a miRNA or miRNA*), such as in a complex mixture of nucleic acids. Stringent conditions are sequence-dependent and will be different in different circumstances. Stringent conditions may be selected to be about 5 to 10° C. lower than the thermal melting point (Tm) for the specific sequence at a defined ionic strength pH. The Tm may be the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0 M sodium ion, such as about 0.01 to 1.0 M sodium ion concentration (or other salts) at pH 7.0 to 8.3 and the temperature is at least about 20° C. for short probes (e.g., about 10-35 nucleotides) and up to 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.; or 6×SSPE, 10% formamide, 0.01%, Tween 20, 0.1×TE buffer, 0.5 mg/ml BSA, 0.1 mg/ml herring sperm DNA, incubating at 42° C. with wash in 05×SSPE and 6×SSPE at 45° C.

The term “antisense”, as used herein, refers to nucleotide sequences which are complementary to a specific DNA or RNA sequence. The term “antisense strand” is used in reference to a nucleic acid strand that is complementary to the “sense” strand.

Residues in two or more polynucleotides are said to “correspond” to each other if the residues occupy an analogous position in the polynucleotide structures. It is well known in the art that analogous positions in two or more polynucleotides can be determined by aligning the polynucleotide sequences based on nucleic acid sequence or structural similarities. Such alignment tools are well known to the person skilled in the art and can be, for example, obtained on the World Wide Web, for example, ClustalW or Align using standard settings, preferably for Align EMBOSS::needle, Matrix: Blosum62, Gap Open 10.0, Gap Extend 0.5.

The term “level”, as used herein, refers to an amount (measured for example in grams, mole, or counts such as ion or fluorescence counts) or concentration (e.g. absolute or relative concentration) of the miRNAs described herein, in particular of the miRNAs having, comprising, essentially consisting of, or consisting of a nucleotide sequence selected from the group consisting of SEQ ID NO: 1 to SEQ ID NO: 24.

The term “level”, as used herein, also comprises scaled, normalized, or scaled and normalized amounts or values. Preferably, the level determined herein is the expression level.

The term “sensitivity”, as used herein, refers to the number of true positive patients (%) with regard to the number of all patients (100%). The patients may be subjects having AD. The sensitivity is calculated by the following formula: Sensitivity=TP/(TP+FN) (TP=true positives; FN=false negatives).

The term “specificity”, as used herein, relates to the number of true negative patients (%) with regard to the number of all healthy subjects (100%). The specificity is calculated by the following formula: Specificity=TN/(TN+FP) (TN=true negatives; FP=false positives).

The term “accuracy”, as used herein, means a statistical measure for the correctness of classification or identification of sample types. The accuracy is the proportion of true results (both true positives and true negatives).

The result of each analysis group is usually calculated from a plurality of isolated samples, i.e. from at least 2 isolated samples, preferably from between 2 and 20, more preferably from between 10 and 60, and even more preferably from between 50 and 100 isolated samples, e.g. selected from the group consisting of subjects not suffering from AD (i.e. subjects being healthy with respect to AD) and subjects suffering from AD. The methods of the present invention can be carried out in combination with other methods for determining AD in a patient to increase the overall sensitivity and/or specificity. The calculation/detection of the level of the miRNAs mentioned herein allows the determination of AD in a patient. In particular, the determination of Alzheimer's Disease comprises diagnosing whether the patient suffers from Alzheimer's Disease, determining whether Alzheimer's Disease is present in the patient, determining whether Alzheimer's Disease is absent in the patient, and/or determining the course of Alzheimer's Disease in the patient.

The term “AUC”, as used herein, relates to an abbreviation for the area under a curve. In particular, it refers to the area under a Receiver Operating Characteristic (ROC) curve. The term “Receiver Operating Characteristic (ROC) curve”, as used herein, refers to a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve is a measure for the accuracy of a diagnostic test (the larger the area the better, optimum is 1, a random test would have a ROC curve lying on the diagonal with an area of 0.5 (see, for reference, for example, J P. Egan. Signal Detection Theory and ROC Analysis).

The term “Alzheimer's Disease (AD)”, as used herein, refers to a neurodegenerative disease. It is the most common form of dementia. Alzheimer's Disease is characterized by loss of neurons and synapses in the cerebral cortex and certain subcortical regions and leads to a gross degeneration in these regions. In Alzheimer's Disease, protein misfolding and aggregation (formation of so-called “plaques”) in the brain is caused by accumulation of abnormally folded A-beta and tau proteins in the affected tissues. The most common early symptom is difficulty in remembering recent events (short-term memory loss). As the disease advances, symptoms can include problems with language, disorientation, mood swings, loss of motivation, not managing self-care and behavioral issues. As a person's condition declines, they often withdraw from family and society. Gradually, bodily functions are lost, ultimately leading to death. AD can be difficult to diagnose accurately. A probable diagnosis is based on the history of the illness and cognitive testing with medical imaging and blood tests to rule out other possible causes. Initial symptoms are often mistaken for normal aging. Examination of the brain tissue (post mortem) is needed for a definite diagnosis. Some diagnostic tests based on biomarkers such as miRNAs are known. The present inventors, however, found new Alzheimer miRNA signatures that hold in different countries and for groups with different ethnical background. Thus, said miRNA signatures allow a reliable determination of AD in a patient. In particular, the new signatures allow to measure cohorts from different countries, which improves the translational process in different aspects. First, required cohort sizes can be obtained more easy since patients from different countries can be included. Second, the regulatory processes can be simplified to a certain aspect. Third, the same assay format and layout can be used in different countries, resulting in lower cost. In the same direction, the same computational solutions can be applied.

The term “determining Alzheimer's Disease in a patient”, as used herein, preferably covers diagnosing whether the patient suffers from Alzheimer's Disease, determining whether Alzheimer's Disease is present in the patient, determining whether Alzheimer's Disease is absent in the patient, and/or determining the course of Alzheimer's Disease in the patient.

The term “diagnosing a patient (suspected of having AD) as having Alzheimer' disease (AD)”, as used herein, means determining whether a patient suffers from AD. Thus, the patient may be diagnosed as suffering from AD.

The term “determining the presence of AD in a patient (suspected of having AD)”, as used herein, means determining whether a patient shows signs of AD. Thus, it may be determined, whether AD is present in the patient.

The term “determining the absence of AD in a patient (suspected of having AD)”, as used herein, means determining whether a patient does not show signs of AD. Thus, it may be determined, whether AD is absent in the patient.

The term “determining whether the patient is at risk for developing AD”, as used herein, means determining whether a patient will show signs of AD, in particular in the future, e.g. within the next 1, 2, 3, 4, or 5 years. Thus, it may be determined whether the patient has a predisposition to develop AD or will likely develop AD.

The term “determining the course of Alzheimer's Disease (AD) in a patient”, as used herein, means determining the development of AD over time, e.g. whether AD worsens in the patient, does not worsen/is stable in the patient, or improves in the patient over time. In particular, the patient to be tested is a patient having/suffering from AD.

The term “diagnosis”, as used herein, refers to the process of determining a possible disease or disorder and, therefore, is a process attempting to define the (clinical) condition of a patient. The level of miRNAs determined according to the present invention correlates with the (clinical) condition of a patient. Preferably, the diagnosis comprises (i) determining the occurrence of AD, especially in an (very) early phase of the disease, (ii) staging of AD, (iii) measuring the response of a patient with AD to therapeutic intervention, and/or (iv) segmentation of a patient suffering from AD.

The term “patient”, as used herein, refers to any subject for whom it is desired to know whether she or he suffers from/has Alzheimer's Disease (AD) or not.

Specifically, the term “patient”, as used herein, refers to a subject suspected to be affected by AD. The patient may be diagnosed to be affected by AD, i.e. diseased, or may be diagnosed to be not affected by AD, i.e. healthy with respect to AD. In addition, it may be determined whether AD is present or absent in the patient.

The term “patient”, as used herein, also refers to a subject that is affected by AD, i.e. diseased. The patient may be retested for AD and may be diagnosed to be still affected by AD, e.g. by a mild form or an advanced/severe form of AD.

It may further be determined whether AD worsens in the patient, is stable in the patient, or improves in the patient over time. For example, it may be determined whether a patient suffering from a mild form of AD has developed an advanced/severe form of AD over time. The term “patient” as used herein, also refers to any subject for whom it is desired to know whether she or he may/will develop AD (in the future). Specifically, the term “patient, as used herein, refers to a subject who has a predisposition to develop AD or will likely develop AD. It may be determined whether the patient has a risk to develop AD/is at risk for the development of AD or not.

It should be noted that a patient that is diagnosed as not suffering from AD, i.e. as being healthy with respect to AD, may possibly suffer from another disease not tested/known.

The patient may be any mammal, including both a human and another mammal, e.g. an animal such as a rabbit, mouse, rat, or monkey. Human subjects as patients are particularly preferred.

The term “(control) subject”, as used herein, refers to a subject known to be not affected by AD (negative control), i.e. healthy with respect to AD. The term “(control) subject”, as used herein, also refers to a subject known to be affected by AD, i.e. diseased. Said (control) subject may have developed an advanced form of AD.

It should be noted that a (control) subject which is known as not suffering from AD, i.e. as being healthy with respect to AD, may possibly suffer from another disease not tested/known. The (control) subject may be any mammal, including both a human and another mammal, e.g. an animal such as a rabbit, mouse, rat, or monkey. Human (control) subjects are particularly preferred.

The term “treatment”, in particular “therapeutic treatment”, as used herein, refers to any therapy which improves the health status and/or prolongs (increases) the lifespan of a patient. Said therapy may eliminate the disease in a patient, arrest or slow the development of a disease in a patient, inhibit or slow the development of a disease in a patient, decrease the frequency or severity of symptoms in a patient, and/or decrease the recurrence in a patient who currently has or who previously has had a disease. In case of Alzheimer's Disease (AD), the (therapeutic) treatment includes, but is not limited to, administration of a drug, cognitive training, ergotherapy, and psychotherapy. The drug may be selected from the group consisting of antidementives, antidepressants, and neuroleptics.

The term “blood sample”, as used herein, refers to any blood sample which allows the determination of Alzheimer's Disease in a patient and/or which can be used for control purposes. In particular, the term “blood sample”, as used herein, encompasses whole blood or a blood fraction such as a blood cell/cellular fraction, serum, or plasma. It is preferred that the whole blood, serum, or plasma sample has a volume of between 0.01 and 20 ml, more preferably of between 0.1 and 10 ml, even more preferably of between 0.5 and 8 ml and most preferably of between 1 and 5 ml.

Said blood sample may be provided by removing blood from a patient or (control) subject, but may also be provided by using a previously isolated sample. For example, a blood sample may be taken from a patient or (control) subject by conventional blood collection techniques.

The blood sample may further be obtained from a patient or (control) subject prior to the initiation of a therapeutic treatment, during the therapeutic treatment, and/or after the therapeutic treatment. If the blood sample is obtained from at least one (control) subject, e.g. from at least 1, 2, 3, 4, 5, 6, 7, 8, 9, 10, 20, 30, 40, 50, 60, 70, 80, 90, 100, 150, 200, 250, 300, 400, 500, or 1,000 (control) subject(s), it is designated as “reference blood sample”. Preferably, the reference blood sample is from the same source than the blood sample of the patient to be tested, e.g. both are whole blood samples or blood cell/cellular fractions. It is further preferred that both are from the same species, e.g. from a human. It is also (alternatively or additionally) preferred that the measurements of the reference blood sample of the (control) subject and the blood sample of the patient to be tested are identical, e.g. both have an identical volume. It is particularly preferred that the reference blood sample and the blood sample are from (control) subjects/patients of the same sex and similar age.

It is preferred that the whole blood sample is collected by means of a blood collection tube. It is, for example, collected in a PAXgene Blood RNA tube, in a Tempus Blood RNA tube, in an EDTA-tube, in a Na-citrate tube, Heparin-tube or in a ACD-tube (Acid citrate dextrose). Preferably, when the whole blood sample is collected, the RNA-fraction, especially the miRNA fraction, may be protected/guarded against degradation. For this purpose special collection tubes (e.g. PAXgene Blood RNA tubes from Preanalytix, Tempus Blood RNA tubes from Applied Biosystems) or additives (e.g. RNAlater from Ambion, RNAsin from Promega), that stabilize the RNA fraction and/or the miRNA fraction, may be employed.

It is also preferred that the whole blood sample is collected by means of a bloodspot technique, e.g. using a Mitra Microsampling Device. This technique requires smaller sample volumes, typically 45-60 μl for humans or less. For example, the whole blood may be extracted from the patient via a finger prick with a needle or lancet. Thus, the whole blood sample may have the form of a blood drop. Said blood drop is then placed on an absorbent probe, e.g. a hydrophilic polymeric material such as cellulose, which is capable of absorbing the whole blood. Once sampling is complete, the blood spot is dried in air before transferring or mailing to labs for processing. Because the blood is dried, it is not considered hazardous. Thus, no special precautions need be taken in handling or shipping. Once at the analysis site, the desired components, e.g. miRNAs, are extracted from the dried blood spots into a supernatant which is then further analyzed. This technique is suitable for monitoring patients having AD or being at risk for AD at home (on a home care/home sampling basis) or for screening purposes.

The term “blood cell/cellular fraction”, as used herein, refers to a blood cell/cellular portion which has been produced from whole blood by removing the extracellular fraction (serum and/or plasma). In other words, the blood cell/cellular fraction is depleted of the extracellular blood components, such as serum and/or plasma. Preferably, the blood cell/cellular portion comprises/essentially consists of/consists of erythrocytes, leukocytes, and/or thrombocytes, e.g. erythrocytes, leukocytes, and thrombocytes.

In one embodiment, the blood sample is a blood cell/cellular fraction. Preferably, the blood cell/cellular fraction comprises/essentially consists of/consists of erythrocytes, leukocytes, and/or thrombocytes, e.g. erythrocytes, leukocytes, and thrombocytes.

In one alternative embodiment, the blood sample is a blood cell sample. Preferably, the blood cell sample comprises/essentially consists of/consists of erythrocytes, leukocytes, and/or thrombocytes, e.g. erythrocytes, leukocytes, and thrombocytes.

In one another alternative embodiment, the blood sample is a blood cell preparation derived from whole blood.

The term “blood cell preparation derived from a whole blood sample”, as used herein, refers to a preparation of a whole blood sample that comprises/essentially consists of/consists of blood cells (erythrocytes, leukocytes, and/or thrombocytes, e.g. erythrocytes, leukocytes, and thrombocytes).

Blood cell preparations derived from a whole sample comprising/essentially consisting of/consisting of erythrocytes, leukocytes, and/or thrombocytes, e.g. erythrocytes, leukocytes, and thrombocytes, are preferably obtained from processing of whole blood samples collected in PAXgene Blood RNA Tubes, Tempus Blood RNA Tubes, EDTA-tubes, Na-citrate tubes or Heparin-tubes maintaining or substantially maintaining the initial cellular distribution (blood cell composition) of the whole blood sample. It is preferred that the whole blood sample is collected, e.g. in a PAXgene RNA tube, and processed according to the manufacturers protocol resulting in a blood cell preparation comprising/essentially consisting of/consisting of erythrocytes, leukocytes, and/or thrombocytes, e.g. erythrocytes, leukocytes, and thrombocytes, from which total RNA (comprising the short RNA fraction including the miRNA fraction) is isolated and which is used for determining the miRNA level in said sample according to the present invention.

In another embodiment of the invention, the blood cell preparation derived from a whole blood sample comprising/essentially consisting of/consisting of erythrocytes, leukocytes, and/or thrombocytes, e.g. erythrocytes, leukocytes, and thrombocytes, is obtained from processing of a whole blood sample collected in PAXgene Blood RNA Tubes, Tempus Blood RNA Tubes, EDTA-tubes, Na-citrate tubes or Heparin-tubes not necessarily maintaining or not necessarily substantially maintaining the initial cellular distribution (blood cell composition) of the whole blood sample.

With respect to the blood cellular fraction or blood cell preparation comprising/essentially consisting of/consisting of erythrocytes, leukocytes, and thrombocytes, it should be noted that the determined miRNA level represents the (mathematical) average of the levels of the miRNAs in the mixture of erythrocytes, leukocytes, and thrombocytes.

The term “total RNA” as used herein relates to the isolated RNA comprising the miRNA-fraction present in a blood sample, e.g. a blood cell preparation derived from a whole blood sample. Preferably, the total RNA according to the present invention contains the miRNA-fraction or contains a miRNA-enriched fraction of the isolated RNA. For example, the total RNA (comprising the miRNA-fraction or miRNA-enriched fraction) is obtained by lysis (e.g. Trizol) of the blood cells in the blood cell preparation, followed by RNA purification e.g. by phenol/chloroform extraction and/or separation based techniques (e.g. glass fiber filter column, silica-membrane column). Examples of kits for RNA isolation and purification include the miRNeasy Kits (Qiagen), PAXgene Blood miRNA Kit (Qiagen), mirVana PARIS Kit (Life Technologies), PARIS Kit (Life Technologies), Tempus Spin RNA Isolation Kit (Life Technologies).

In the context of the present invention, the term “kit of parts (in short: kit)” is understood to be any combination of at least some of the components identified herein, which are combined, coexisting spatially, to a functional unit, and which can contain further components. Said kit may allow point-of-care testing (POCT).

The term “point-of-care testing (POCT)”, as used herein, refers to a medical diagnostic testing at or near the point of care that is the time and place of patient care. This contrasts with the historical pattern in which testing was wholly or mostly confined to the medical laboratory, which entailed sending off specimens away from the point of care and then waiting hours or days to learn the results, during which time care must continue without the desired information. Point-of-care tests are simple medical tests that can be performed at the bedside. The driving notion behind POCT is to bring the test conveniently and immediately to the patient to be tested. This increases the likelihood that the patient, physician, and care team will receive the results quicker, which allows for immediate clinical management decisions to be made. POCT is often accomplished through the use of transportable, portable, and handheld instruments and test kits. Small bench analyzers or fixed equipment can also be used when a handheld device is not available—the goal is to collect the specimen and obtain the results in a very short period of time at or near the location of the patient so that the treatment plan can be adjusted as necessary before the patient leaves the hospital.

### Embodiments of the Invention

Diagnosis of AD by mental testing and medical history analysis is time consuming, expensive, and difficult. MiRNAs have already been suggested as non-invasive biomarkers for the diagnosis of Alzheimer's Disease. In particular, the detection of miRNAs in a biological sample of a patient in order to determine whether the patient suffers from Alzheimer's Disease has already been recommended. However, the diagnostic power of described miRNA based diagnostics tests and the reliability of the data obtained therefrom leaves much to be desired. In particular, the reliable and early diagnosis of AD based on non-invasive molecular biomarkers remains a challenge. Especially, the miRNA signatures suggested only allow the analysis of a specific group of people (regionally limited, country-specific). The present inventors found new Alzheimer signatures that hold in different countries and for groups with different ethnical background. In particular, the new signatures allow to measure cohorts from different countries, which improves the translational process in different aspects. First, required cohort sizes can be obtained more easy since patients from different countries can be included. Second, the regulatory processes can be simplified to a certain aspect. Third, the same assay format and layout can be used in different countries, resulting in lower cost. In the same direction, the same computational solutions can be applied.

Thus, in a first aspect, the present invention relates to a(n) (in vitro) method for determining Alzheimer's Disease in a patient which comprises the steps of:
(i) determining the level (of each) of at least 3 miRNAs, e.g. at least 3, 4, 5, or 6 miRNAs, or 7 miRNAs, comprised in a set in a blood sample from a patient, wherein the first miRNA has a nucleotide sequence according to SEQ ID NO: 1 (hsa-miR-363-3p) or a nucleotide sequence having at least 90%, preferably at least 95%, more preferably at least 99%, i.e. at least 90, 91, 92, 93, 94, 95, 96, 97, 98, or 99%, sequence identity thereto, and
(ii) comparing the level (of each) of the at least 3 miRNAs comprised in the set with a reference level of said at least three miRNAs, wherein the comparison allows to determine Alzheimer's Disease in the patient.

It is preferred that the second and third miRNAs have nucleotide sequences selected from the group consisting of SEQ ID NO: 2 to SEQ ID NO: 18. Alternatively, the nucleotide sequences of the miRNAs according to SEQ ID NO: 2 to SEQ ID NO: 18 are nucleotide sequences having at least 90%, preferably at least 95%, more preferably at least 99%, i.e. at least 90, 91, 92, 93, 94, 95, 96, 97, 98, or 99%, sequence identity thereto.

It is more preferred that the second miRNA has a nucleotide sequence according to SEQ ID NO: 2 (hsa-miR-28-3p) or SEQ ID NO: 3 (hsa-let-7e-5p) and wherein the third miRNA has a nucleotide sequence selected from the group consisting of SEQ ID NO: 3 to SEQ ID NO: 18 under the proviso that the second and third miRNAs are different.

It is even more preferred that the second miRNA has a nucleotide sequence according to SEQ ID NO: 2 and the third miRNA has a nucleotide sequence selected from the group consisting of SEQ ID NO: 3 to SEQ ID NO: 15.

It is also even more preferred that the second miRNA has a nucleotide sequence according to SEQ ID NO: 3 and the third miRNA has a nucleotide sequence selected from the group consisting of SEQ ID NO: 2, SEQ ID NO: 4 to SEQ ID NO: 11, and SEQ ID NO: 16 to SEQ ID NO: 18.

It is particularly preferred that the second miRNA has a nucleotide sequence according to SEQ ID NO: 2 (hsa-miR-28-3p) and the third miRNA has a nucleotide sequence according to SEQ ID NO: 3 (hsa-let-7e-5p).

It is most preferred that the at least three miRNAs are comprised in a set selected from the group consisting of:
(a) SEQ ID NO: 1, SEQ ID NO: 2, and SEQ ID NO: 9,
(b) SEQ ID NO: 1, SEQ ID NO: 2, and SEQ ID NO: 4,
(c) SEQ ID NO: 1, SEQ ID NO: 2, and SEQ ID NO: 5,
(d) SEQ ID NO: 1, SEQ ID NO: 2, and SEQ ID NO: 6,
(e) SEQ ID NO: 1, SEQ ID NO: 2, SEQ ID NO: 6, and SEQ ID NO: 9,
(f) SEQ ID NO: 1, SEQ ID NO: 2, SEQ ID NO: 5, and SEQ ID NO: 12,
(g) SEQ ID NO: 1, SEQ ID NO: 2, SEQ ID NO: 3, SEQ ID NO: 6, SEQ ID NO: 8, and SEQ ID NO: 10,
(h) SEQ ID NO: 1, SEQ ID NO: 2, SEQ ID NO: 3, SEQ ID NO: 6, and SEQ ID NO: 8,
(i) SEQ ID NO: 1, SEQ ID NO: 2, SEQ ID NO: 5, SEQ ID NO: 10, SEQ ID NO: 12, and SEQ ID NO: 13,
(j) SEQ ID NO: 1, SEQ ID NO: 2, SEQ ID NO: 3, SEQ ID NO: 5, SEQ ID NO: 6, and SEQ ID NO: 11,
(k) SEQ ID NO: 1, SEQ ID NO: 2, SEQ ID NO: 3, SEQ ID NO: 4, SEQ ID NO: 7, SEQ ID NO: 8, and SEQ ID NO: 11,
(l) SEQ ID NO: 1, SEQ ID NO: 2, SEQ ID NO: 12, and SEQ ID NO: 13,
(m) SEQ ID NO: 1, SEQ ID NO: 2, SEQ ID NO: 5, SEQ ID NO: 9, and SEQ ID NO: 12,
(n) SEQ ID NO: 1, SEQ ID NO: 2, SEQ ID NO: 7, SEQ ID NO: 9, and SEQ ID NO: 12,
(o) SEQ ID NO: 1, SEQ ID NO: 2, SEQ ID NO: 9, SEQ ID NO: 10, and SEQ ID NO: 12,
(p) SEQ ID NO: 1, SEQ ID NO: 2, and SEQ ID NO: 12,
(q) SEQ ID NO: 1, SEQ ID NO: 2, and SEQ ID NO: 14,
(r) SEQ ID NO: 1, SEQ ID NO: 2, SEQ ID NO: 9, and SEQ ID NO: 12,
(s) SEQ ID NO: 1, SEQ ID NO: 2, and SEQ ID NO: 15,
(t) SEQ ID NO: 1, SEQ ID NO: 3, SEQ ID NO: 4, SEQ ID NO: 7, and SEQ ID NO: 11,
(u) SEQ ID NO: 1, SEQ ID NO: 3, SEQ ID NO: 5, SEQ ID NO: 8, SEQ ID NO: 9, and SEQ ID NO: 10,
(v) SEQ ID NO: 1, SEQ ID NO: 3, SEQ ID NO: 4, SEQ ID NO: 5, SEQ ID NO: 7, SEQ ID NO: 11, and SEQ ID NO: 16,
(w) SEQ ID NO: 1, SEQ ID NO: 3, SEQ ID NO: 5, SEQ ID NO: 6, SEQ ID NO: 8, SEQ ID NO: 16, and SEQ ID NO: 17,
(x) SEQ ID NO: 1, SEQ ID NO: 3, SEQ ID NO: 5, SEQ ID NO: 8, SEQ ID NO: 16, SEQ ID NO: 17, and SEQ ID NO: 18, and
(y) SEQ ID NO: 1, SEQ ID NO: 3, SEQ ID NO: 6, SEQ ID NO: 7, SEQ ID NO: 8, SEQ ID NO: 16, and SEQ ID NO: 17.

It should be noted that the at least three miRNAs have nucleotide sequences as shown in the sets according to (a) to (y). For example, the set according to (a) comprises the miRNA having a nucleotide sequence according to SEQ ID NO: 1, the miRNA having a nucleotide sequence according to SEQ ID NO: 2, and the miRNA having a nucleotide sequence according to SEQ ID NO: 9.

The patient who's miRNA level is determined may be a patient suspected of suffering from/having AD or a patient suffering from/having AD. In the latter case, the patient may be retested for AD (e.g. after a period of time).

The reference level may be any level which allows to determine AD in the patient or not. It may be obtained from a (control) subject (i.e. a subject different from the patient to be tested/diagnosed) or from the same patient. In the latter case, the patient may be retested for AD, e.g. in the form of a longitudinal monitoring. It may be determined that the patient is now affected by AD or still not affected by AD.

Preferably, the reference level is the level determined by measuring at least one reference blood sample from

at least one healthy subject, or
at least one subject having Alzheimer's Disease.

It is preferred that the reference level is the level determined by measuring at least one reference blood sample, e.g. at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 250, 300, 400, 500, or 1.000 reference blood sample(s), isolated from at least one (control) subject being healthy, i.e. not suffering from AD, e.g. from at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 250, 300, 400, 500, or 1.000 (control) subject(s) being healthy, i.e. not suffering from AD. It is more preferred that the reference level is the level determined by measuring between 2 and 500 reference blood samples isolated from between 2 and 500 subjects being healthy, i.e. not suffering from AD. It is even more preferred that the reference level is determined by measuring between 50 and 500 reference blood samples isolated from between 50 and 500 subjects being healthy, i.e. not suffering from AD. It is most preferred that the reference level is determined by measuring between 100 and 500 reference blood samples isolated from between 100 and 500 subjects being healthy, i.e. not suffering from AD.

Alternatively, it is preferred that the reference level is the level determined by measuring at least one reference blood sample, e.g. at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 250, 300, 400, 500, or 1.000 reference blood sample(s), isolated from at least one (control) subject having AD, e.g. from at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 250, 300, 400, 500, or 1.000 (control) subject(s) having AD. It is more preferred that the reference level is the level determined by measuring between 2 and 500 reference blood samples isolated from between 2 and 500 subjects having AD. It is even more preferred that the reference level is determined by measuring between 50 and 500 reference blood samples isolated from between 50 and 500 subjects having AD. It is most preferred that the reference level is determined by measuring between 100 and 500 reference blood samples isolated from between 100 and 500 subjects having AD.

It is practicable to take one reference blood sample per subject for analysis. If additional reference blood samples are required, e.g. to determine the reference level in different reference blood samples, the same subject may be (re)tested. Said reference level may be an average reference level. It may be determined by measuring reference levels and calculating the “average” value (e.g. mean, median or modal value) thereof. It is preferred that the reference blood sample is from the same source (e.g. blood cell sample) than the blood sample isolated from the patient. It is further preferred that the reference level is obtained from a subject of the same gender (e.g. female or male) and/or of a similar age/phase of life (e.g. adults or elderly) than the patient to be tested or diagnosed.

In one embodiment, the determination of Alzheimer's Disease (AD) comprises diagnosing whether the patient (suspected of having AD) suffers from AD or not. In this case, the reference level may be any level which allows to determine whether the patient (suspected of having AD) suffers from AD or not. It may be obtained from a (control) subject (i.e. a subject different from the patient to be tested/diagnosed) or from the same patient. In the latter case, the patient may be retested for AD. In particular, the reference level is the level determined by measuring at least one reference blood sample from at least one healthy subject (see above).

Preferably,

the level of the miRNA having a nucleotide sequence selected from the group consisting of SEQ ID NO: 1, SEQ ID NO: 3, and SEQ ID NO: 18 is above the reference level (determined by measuring at least one reference blood sample from at least one healthy subject) which indicates that the patient suffers from Alzheimer's Disease, and/or
the level of the miRNA having a nucleotide sequence selected from the group consisting of SEQ ID NO: 2 and SEQ ID NO: 4 to SEQ ID NO: 17 is below the reference level (determined by measuring at least one reference blood sample from at least one healthy subject) which indicates that the patient suffers from Alzheimer's Disease.

Thus, for example, when the level of the miRNAs comprised in set (a), namely the miRNAs having the nucleotide sequences according to SEQ ID NO: 1, SEQ ID NO: 2, and SEQ ID NO: 9, is determined and when the level of the miRNA having the nucleotide sequence according to SEQ ID NO: 1 is above the reference level (determined by measuring at least one reference blood sample from at least one healthy subject) and the level of the miRNAs having the nucleotide sequences according to SEQ ID NO: 2 and SEQ ID NO: 9 is below the reference level (determined by measuring at least one reference blood sample from at least one healthy subject), this indicates that the patient suffers from AD.

In particular, the level of the miRNA is at least 0.4-fold, at least 0.5-fold, at least 0.6-fold or at least 0.7-fold, preferably at least 0.8-fold or at least 0.9-fold, more preferably at least 1.2-fold or at least 1.5-fold, and even more preferably at least 2.0-fold or at least 3.0-fold below/above the reference level. For example, the level of the miRNA is at least 0.4-fold, at least 0.5-fold, at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

In one another embodiment, the determination of Alzheimer's Disease (AD) comprises determining whether AD is present in the patient (suspected of having AD). In this case, the reference level may be any level which allows to determine whether AD is present in the patient (suspected of having AD). It may be obtained from a (control) subject (i.e. a subject different from the patient to be tested) or from the same patient. In the latter case, the patient may be retested for AD. In particular, the reference level is the level determined by measuring at least one reference blood sample from at least one healthy subject (see above).

Preferably,

the level of the miRNA having a nucleotide sequence selected from the group consisting of SEQ ID NO: 1, SEQ ID NO: 3, and SEQ ID NO: 18 is above the reference level (determined by measuring at least one reference blood sample from at least one healthy subject) which indicates that Alzheimer's Disease is present in the patient, and/or
the level of the miRNA having a nucleotide sequence selected from the group consisting of SEQ ID NO: 2 and SEQ ID NO: 4 to SEQ ID NO: 17 is below the reference level (determined by measuring at least one reference blood sample from at least one healthy subject) which indicates that Alzheimer's Disease is present in the patient.

In particular, the level of the miRNA is at least 0.4-fold, at least 0.5-fold, at least 0.6-fold or at least 0.7-fold, preferably at least 0.8-fold or at least 0.9-fold, more preferably at least 1.2-fold or at least 1.5-fold, and even more preferably at least 2.0-fold or at least 3.0-fold below/above the reference level. For example, the level of the miRNA is at least 0.4-fold, at least 0.5-fold, at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

In one another embodiment, the determination of Alzheimer's Disease (AD) comprises determining whether AD is absent in the patient (suspected of having AD). In this case, the reference level may be any level which allows to determine whether AD is absent in the patient (suspected of having AD). It may be obtained from a (control) subject (i.e. a subject different from the patient to be tested) or from the same patient. In the latter case, the patient may be retested for AD. In particular, the reference level is the level determined by measuring at least one reference blood sample from at least one healthy subject (see above).

Preferably,

the level of the miRNA having a nucleotide sequence selected from the group consisting of SEQ ID NO: 1, SEQ ID NO: 3, and SEQ ID NO: 18 is comparable with the reference level (determined by measuring at least one reference blood sample from at least one healthy subject) which indicates that Alzheimer's Disease is absent in the patient,
the level of the miRNA having a nucleotide sequence selected from the group consisting of SEQ ID NO: 2 and SEQ ID NO: 4 to SEQ ID NO: 17 is comparable with the reference level (determined by measuring at least one reference blood sample from at least one healthy subject) which indicates that Alzheimer's Disease is absent in the patient,
the level of the miRNA having a nucleotide sequence selected from the group consisting of SEQ ID NO: 1, SEQ ID NO: 3, and SEQ ID NO: 18 is below the reference level (determined by measuring at least one reference blood sample from at least one subject having Alzheimer's Disease) which indicates that Alzheimer's Disease is absent in the patient, and/or
the level of the miRNA having a nucleotide sequence selected from the group consisting of SEQ ID NO: 2 and SEQ ID NO: 4 to SEQ ID NO: 17 is above the reference level (determined by measuring at least one reference blood sample from at least one subject having Alzheimer's Disease) which indicates that Alzheimer's Disease is absent in the patient.

A level which is “comparable with” the reference level in this respect means that the level is no more than 15%, preferably no more than 10%, more preferably no more than 5%, above the reference level or the level is no more than 15%, preferably no more than 10%, more preferably no more than 5%, below the reference level.

Alternatively, a level which is comparable with the reference level in this respect means that the detected level variation is within the accuracy of a measurement. The accuracy of a measurement depends on the measurement method used.

In particular, the level of the miRNA is at least 0.4-fold, at least 0.5-fold, at least 0.6-fold or at least 0.7-fold, preferably at least 0.8-fold or at least 0.9-fold, more preferably at least 1.2-fold or at least 1.5-fold, and even more preferably at least 2.0-fold or at least 3.0-fold below/above the reference level. For example, the level of the miRNA is at least 0.4-fold, at least 0.5-fold, at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

In one another embodiment, the determination of Alzheimer's Disease (AD) comprises determining whether the patient is at risk for developing AD. In this case, the reference level may be any level which allows to determine whether the patient is at risk for developing AD or not. It may be obtained from a (control) subject (i.e. a subject different from the patient to be tested) or from the same patient. In the latter case, the patient may be retested for AD. In particular, the reference level is the level determined by measuring at least one reference blood sample from at least one healthy subject (see above). This determination may have the form of a screening method. Said screening method allows an early detection of AD and, thus, the identification of patients having a predisposition or a likelihood (e.g. on the basis of their physical and/or physiological conditions) to develop AD, e.g. in the near future, such as within the next 1, 2, 3, 4, or 5 years.

Preferably,

the level of the miRNA having a nucleotide sequence selected from the group consisting of SEQ ID NO: 1, SEQ ID NO: 3, and SEQ ID NO: 18 is above the reference level (determined by measuring at least one reference blood sample from at least one healthy subject) which indicates that the patient is at risk for developing Alzheimer's Disease, and/or
the level of the miRNA having a nucleotide sequence selected from the group consisting of SEQ ID NO: 2 and SEQ ID NO: 4 to SEQ ID NO: 17 is below the reference level (determined by measuring at least one reference blood sample from at least one healthy subject) which indicates that the patient is at risk for developing Alzheimer's Disease.

In particular, the level of the miRNA is at least 0.4-fold, at least 0.5-fold, at least 0.6-fold or at least 0.7-fold, preferably at least 0.8-fold or at least 0.9-fold, more preferably at least 1.2-fold or at least 1.5-fold, and even more preferably at least 2.0-fold or at least 3.0-fold below/above the reference level. For example, the level of the miRNA is at least 0.4-fold, at least 0.5-fold, at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

In another embodiment, the determination of Alzheimer's Disease (AD) comprises determining the course of AD in the patient (having AD). In this case, the reference level may be any level which allows to determine the course of AD in the patient (having AD). It may be obtained from a (control) subject (i.e. a subject different from the patient to be tested/diagnosed) or from the same patient. In the latter case, the patient may be retested for AD. It may be determined whether AD worsens in the patient, whether AD does not worsen/is stable in the patient, or whether AD improves in the patient. In particular, the reference level is the level determined by measuring at least one reference blood sample from at least one subject having AD (see above).

Especially, said determining comprises determining the level (of each) of the at least three miRNAs comprised in the set in the blood sample at a first point in time and in at least one further blood sample at a later point in time and comparing said levels determined at the different time points.

Preferably,

the level of the miRNA having a nucleotide sequence selected from the group consisting SEQ ID NO: 1, SEQ ID NO: 3, and SEQ ID NO: 18 which
(i) increases over time indicates that Alzheimer's Disease worsens in the patient,
(ii) does not change over time indicates that Alzheimer's Disease does not worsen/is stable in the patient, or
(iii) decreases over time indicates that Alzheimer's Disease improves in the patient,
   and/or
   the level of the miRNA having a nucleotide sequence selected from the group consisting of SEQ ID NO: 2 and SEQ ID NO: 4 to SEQ ID NO: 17 which
(i) decreases over time indicates that Alzheimer's Disease worsens in the patient,
(ii) does not change over time indicates that Alzheimer's Disease does not worsen/is stable in the patient, or
(iii) increases over time indicates that Alzheimer's Disease improves in the patient.

As mentioned above, the detection of a decrease/an increase (dependent on the miRNA detected) of the level over time indicates that AD worsens in the patient. Preferably, said decrease/increase is at least 0.4-fold, at least 0.5-fold, at least 0.6-fold or at least 0.7-fold over time. More preferably, said decrease/increase is at least 0.8-fold or at least 0.9-fold over time. Even more preferably, said decrease/increase is at least 1.2-fold or at least 1.5-fold over time. Most preferably, said decrease/increase is at least 2.0-fold or at least 3.0-fold over time. For example, said decrease/increase may be determined over 1 year (12 months) or over 2 years (24 months).

As mentioned above, a level which does not change over time indicates that AD does not worsen/is stable in the patient. “Does not change over time” in this respect may mean that the level varies over time between 0 and <20%, e.g. 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 19.9, 19.99, or 19.999%. “Does not change over time” in this respect may also mean that the detected level variation is within the accuracy of a measurement. The accuracy of a measurement depends on the measurement method used. Preferably, the level is constant over time.

As mentioned above, the detection of an increase/a decrease (dependent on the miRNA detected) of the level over time indicates that AD improves in the patient. Preferably, said increase/decrease is at least 0.4-fold, at least 0.5-fold, at least 0.6-fold or at least 0.7-fold over time. More preferably, said increase/decrease is at least 0.8-fold or at least 0.9-fold over time. Even more preferably, said increase/decrease is at least 1.2-fold or at least 1.5-fold over time. Most preferably, said increase/decrease is at least 2.0-fold or at least 3.0-fold over time. For example, said increase/decrease may be determined over 1 year (12 months) or over 2 years (24 months).

The time period between the first point in time and the later point(s) in time preferably amounts to at least 1 day, at least 2 days, at least 3 days, at least 4 days, at least 5 days, at least 6 days, at least 7 days (1 week), at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months (1 year), at least 24 months (2 years), at least 3 years, at least 4 years, at least 5 years, at least 6 years, at least 7 years, at least 8 years, at least 9 years, or at least 10 years. For example, the patient may be routinely checked, e.g. once or twice a year. The patient may be (re)tested at 2, 3, 4, 5, 6 7, 8, 9, or 10 time points (first point in time and further point(s) in time).

In addition to the determination of the course of AD, the treatment of this disease can be monitored. It is namely preferred that the patient receives or has received a treatment, in particular therapeutic treatment, of AD during the determination of the course of AD. The treatment of AD may be selected from the group consisting of the administration of a drug, cognitive training, ergotherapy, and psychotherapy. The drug may be selected from the group consisting of antidementives, antidepressants, and neuroleptics.

The patient may receive a treatment during the complete determination/monitoring process (e.g. the administration of a drug) or may receive a treatment before, at, or after a first point in time (e.g. the administration of a drug) and may be retested at a later point in time. In particular, said first point in time may be before the initiation of a treatment and said later point in time may be during the treatment and/or after the treatment. If the treatment encompasses the administration of a drug and the patient responds to said treatment, the drug administration may be continued, the dose of the drug may be reduced, or the drug administration may be stopped. If the treatment encompasses the administration of a drug and the patient does not respond to said treatment, the dose of the drug may be increased, the drug may be changed, or the therapy mode may be changed, e.g. from drug administration to cognitive training, ergotherapy, and/or psychotherapy.

As mentioned above, it is preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for Alzheimer's Disease, and/or determining the course of Alzheimer's Disease in the patient. Thus, in view of the above, the method according to the first aspect of the present invention is preferably designated as a method

for diagnosing/determining whether a patient (suspected to be affected by AD) suffers from AD or not,
for determining the presence of AD in a patient (suspected to be affected by AD),
for determining the absence of AD in a patient (suspected to be affected by AD),
for determining the risk for developing AD in a patient, and/or
for determining the course of AD in a patient (having/suffering from AD).

In a second aspect, the present invention relates to a(n) (in vitro) method for determining Alzheimer's Disease in a patient which comprises the steps of:
(i) determining the level (of each) of at least 3 miRNAs, e.g. at least 3, 4, 5, or 6 miRNAs, or 7 miRNAs, in a blood sample isolated from a patient, wherein the at least three miRNAs are comprised in a set selected from the group consisting of:
   (a) SEQ ID NO: 1, SEQ ID NO: 2, and SEQ ID NO: 9,
   (b) SEQ ID NO: 1, SEQ ID NO: 2, and SEQ ID NO: 4,
   (c) SEQ ID NO: 1, SEQ ID NO: 2, and SEQ ID NO: 5,
   (d) SEQ ID NO: 1, SEQ ID NO: 2, and SEQ ID NO: 6,
   (e) SEQ ID NO: 1, SEQ ID NO: 2, SEQ ID NO: 6, and SEQ ID NO: 9,
   (f) SEQ ID NO: 1, SEQ ID NO: 2, SEQ ID NO: 5, and SEQ ID NO: 12,
   (g) SEQ ID NO: 1, SEQ ID NO: 2, SEQ ID NO: 3, SEQ ID NO: 6, SEQ ID NO: 8, and SEQ ID NO: 10,
   (h) SEQ ID NO: 1, SEQ ID NO: 2, SEQ ID NO: 3, SEQ ID NO: 6, and SEQ ID NO: 8,
   (i) SEQ ID NO: 1, SEQ ID NO: 2, SEQ ID NO: 5, SEQ ID NO: 10, SEQ ID NO: 12, and SEQ ID NO: 13,
   (j) SEQ ID NO: 1, SEQ ID NO: 2, SEQ ID NO: 3, SEQ ID NO: 5, SEQ ID NO: 6, and SEQ ID NO: 11,
   (k) SEQ ID NO: 1, SEQ ID NO: 2, SEQ ID NO: 3, SEQ ID NO: 4, SEQ ID NO: 7, SEQ ID NO: 8, and SEQ ID NO: 11,
   (l) SEQ ID NO: 1, SEQ ID NO: 2, SEQ ID NO: 12, and SEQ ID NO: 13,
   (m) SEQ ID NO: 1, SEQ ID NO: 2, SEQ ID NO: 5, SEQ ID NO: 9, and SEQ ID NO: 12,
   (n) SEQ ID NO: 1, SEQ ID NO: 2, SEQ ID NO: 7, SEQ ID NO: 9, and SEQ ID NO: 12,
   (o) SEQ ID NO: 1, SEQ ID NO: 2, SEQ ID NO: 9, SEQ ID NO: 10, and SEQ ID NO: 12,
   (p) SEQ ID NO: 1, SEQ ID NO: 2, and SEQ ID NO: 12,
   (q) SEQ ID NO: 1, SEQ ID NO: 2, and SEQ ID NO: 14,
   (r) SEQ ID NO: 1, SEQ ID NO: 2, SEQ ID NO: 9, and SEQ ID NO: 12,
   (s) SEQ ID NO: 1, SEQ ID NO: 2, and SEQ ID NO: 15,
   (t) SEQ ID NO: 1, SEQ ID NO: 3, SEQ ID NO: 4, SEQ ID NO: 7, and SEQ ID NO: 11,
   (u) SEQ ID NO: 1, SEQ ID NO: 3, SEQ ID NO: 5, SEQ ID NO: 8, SEQ ID NO: 9, and SEQ ID NO: 10,
   (v) SEQ ID NO: 1, SEQ ID NO: 3, SEQ ID NO: 4, SEQ ID NO: 5, SEQ ID NO: 7, SEQ ID NO: 11, and SEQ ID NO: 16,
   (w) SEQ ID NO: 1, SEQ ID NO: 3, SEQ ID NO: 5, SEQ ID NO: 6, SEQ ID NO: 8, SEQ ID NO: 16, and SEQ ID NO: 17,
   (x) SEQ ID NO: 1, SEQ ID NO: 3, SEQ ID NO: 5, SEQ ID NO: 8, SEQ ID NO: 16, SEQ ID NO: 17, and SEQ ID NO: 18, and
   (y) SEQ ID NO: 1, SEQ ID NO: 3, SEQ ID NO: 6, SEQ ID NO: 7, SEQ ID NO: 8, SEQ ID NO: 16, and SEQ ID NO: 17,
   and
(ii) comparing the level (of each) of the at least 3 miRNAs comprised in the set with a reference level of said at least three miRNAs, wherein the comparison allows to determine Alzheimer's Disease in the patient.

It should be noted that the at least three miRNAs have nucleotide sequences as shown in the sets according to (a) to (y). For example, the set according to (a) comprises the miRNA having a nucleotide sequence according to SEQ ID NO: 1, the miRNA having a nucleotide sequence according to SEQ ID NO: 2, and the miRNA having a nucleotide sequence according to SEQ ID NO: 9.

Alternatively, the nucleotide sequences of the miRNAs according to SEQ ID NO: 1 to SEQ ID NO: 18 comprised in the above mentioned sets are nucleotide sequences having at least 90%, preferably at least 95%, more preferably at least 99%, i.e. at least 90, 91, 92, 93, 94, 95, 96, 97, 98, or 99%, sequence identity thereto.

The patient who's miRNA level is determined may be a patient suspected of suffering from/having AD or a patient suffering from/having AD. In the latter case, the patient may be retested for AD (e.g. after a period of time).

The reference level may be any level which allows to determine AD in the patient or not. It may be obtained from a (control) subject (i.e. a subject different from the patient to be tested/diagnosed) or from the same patient. In the latter case, the patient may be retested for AD, e.g. in the form of a longitudinal monitoring. It may be determined that the patient is now affected by AD or still not affected by AD.

Preferably, the reference level is the level determined by measuring at least one reference blood sample from

at least one healthy subject, or
at least one subject having Alzheimer's Disease.

It is preferred that the reference level is the level determined by measuring at least one reference blood sample, e.g. at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 250, 300, 400, 500, or 1.000 reference blood sample(s), isolated from at least one (control) subject being healthy, i.e. not suffering from AD, e.g. from at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 250, 300, 400, 500, or 1.000 (control) subject(s) being healthy, i.e. not suffering from AD. It is more preferred that the reference level is the level determined by measuring between 2 and 500 reference blood samples isolated from between 2 and 500 subjects being healthy, i.e. not suffering from AD. It is even more preferred that the reference level is determined by measuring between 50 and 500 reference blood samples isolated from between 50 and 500 subjects being healthy, i.e. not suffering from AD. It is most preferred that the reference level is determined by measuring between 100 and 500 reference blood samples isolated from between 100 and 500 subjects being healthy, i.e. not suffering from AD.

Alternatively, it is preferred that the reference level is the level determined by measuring at least one reference blood sample, e.g. at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 250, 300, 400, 500, or 1.000 reference blood sample(s), isolated from at least one (control) subject having AD, e.g. from at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 250, 300, 400, 500, or 1.000 (control) subject(s) having AD. It is more preferred that the reference level is the level determined by measuring between 2 and 500 reference blood samples isolated from between 2 and 500 subjects having AD. It is even more preferred that the reference level is determined by measuring between 50 and 500 reference blood samples isolated from between 50 and 500 subjects having AD. It is most preferred that the reference level is determined by measuring between 100 and 500 reference blood samples isolated from between 100 and 500 subjects having AD.

It is practicable to take one reference blood sample per subject for analysis. If additional reference blood samples are required, e.g. to determine the reference level in different reference blood samples, the same subject may be (re)tested. Said reference level may be an average reference level. It may be determined by measuring reference levels and calculating the “average” value (e.g. mean, median or modal value) thereof. It is preferred that the reference blood sample is from the same source (e.g. blood cell sample) than the blood sample isolated from the patient. It is further preferred that the reference level is obtained from a subject of the same gender (e.g. female or male) and/or of a similar age/phase of life (e.g. adults or elderly) than the patient to be tested or diagnosed.

In one embodiment, the determination of Alzheimer's Disease (AD) comprises diagnosing whether the patient (suspected of having AD) suffers from AD or not. In this case, the reference level may be any level which allows to determine whether the patient (suspected of having AD) suffers from AD or not. It may be obtained from a (control) subject (i.e. a subject different from the patient to be tested/diagnosed) or from the same patient. In the latter case, the patient may be retested for AD. In particular, the reference level is the level determined by measuring at least one reference blood sample from at least one healthy subject (see above).

Preferably,

the level of the miRNA having a nucleotide sequence selected from the group consisting of SEQ ID NO: 1, SEQ ID NO: 3, and SEQ ID NO: 18 is above the reference level (determined by measuring at least one reference blood sample from at least one healthy subject) which indicates that the patient suffers from Alzheimer's Disease, and/or
the level of the miRNA having a nucleotide sequence selected from the group consisting of SEQ ID NO: 2 and SEQ ID NO: 4 to SEQ ID NO: 17 is below the reference level (determined by measuring at least one reference blood sample from at least one healthy subject) which indicates that the patient suffers from Alzheimer's Disease.

In particular, the level of the miRNA is at least 0.4-fold, at least 0.5-fold, at least 0.6-fold or at least 0.7-fold, preferably at least 0.8-fold or at least 0.9-fold, more preferably at least 1.2-fold or at least 1.5-fold, and even more preferably at least 2.0-fold or at least 3.0-fold below/above the reference level. For example, the level of the miRNA is at least 0.4-fold, at least 0.5-fold, at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

In one another embodiment, the determination of Alzheimer's Disease (AD) comprises determining whether AD is present in the patient (suspected of having AD). In this case, the reference level may be any level which allows to determine whether AD is present in the patient. It may be obtained from a (control) subject (i.e. a subject different from the patient to be tested) or from the same patient. In the latter case, the patient may be retested for AD. In particular, the reference level is the level determined by measuring at least one reference blood sample from at least one healthy subject (see above).

Preferably,

the level of the miRNA having a nucleotide sequence selected from the group consisting of SEQ ID NO: 1, SEQ ID NO: 3, and SEQ ID NO: 18 is above the reference level (determined by measuring at least one reference blood sample from at least one healthy subject) which indicates that Alzheimer's Disease is present in the patient, and/or
the level of the miRNA having a nucleotide sequence selected from the group consisting of SEQ ID NO: 2 and SEQ ID NO: 4 to SEQ ID NO: 17 is below the reference level (determined by measuring at least one reference blood sample from at least one healthy subject) which indicates that Alzheimer's Disease is present in the patient.

In particular, the level of the miRNA is at least 0.4-fold, at least 0.5-fold, at least 0.6-fold or at least 0.7-fold, preferably at least 0.8-fold or at least 0.9-fold, more preferably at least 1.2-fold or at least 1.5-fold, and even more preferably at least 2.0-fold or at least 3.0-fold below/above the reference level. For example, the level of the miRNA is at least 0.4-fold, at least 0.5-fold, at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

In one another embodiment, the determination of Alzheimer's Disease (AD) comprises determining whether AD is absent in the patient (suspected of having AD). In this case, the reference level may be any level which allows to determine whether AD is absent in the patient (suspected of having AD). It may be obtained from a (control) subject (i.e. a subject different from the patient to be tested/diagnosed) or from the same patient. In the latter case, the patient may be retested for AD. In particular, the reference level is the level determined by measuring at least one reference blood sample from at least one healthy subject (see above).

Preferably,

the level of the miRNA having a nucleotide sequence selected from the group consisting of SEQ ID NO: 1, SEQ ID NO: 3, and SEQ ID NO: 18 is comparable with the reference level (determined by measuring at least one reference blood sample from at least one healthy subject) which indicates that Alzheimer's Disease is absent in the patient,
the level of the miRNA having a nucleotide sequence selected from the group consisting of SEQ ID NO: 2 and SEQ ID NO: 4 to SEQ ID NO: 17 is comparable with the reference level (determined by measuring at least one reference blood sample from at least one healthy subject) which indicates that Alzheimer's Disease is absent in the patient,
the level of the miRNA having a nucleotide sequence selected from the group consisting of SEQ ID NO: 1, SEQ ID NO: 3, and SEQ ID NO: 18 is below the reference level (determined by measuring at least one reference blood sample from at least one subject having Alzheimer's Disease) which indicates that Alzheimer's Disease is absent in the patient, and/or
the level of the miRNA having a nucleotide sequence selected from the group consisting of SEQ ID NO: 2 and SEQ ID NO: 4 to SEQ ID NO: 17 is above the reference level (determined by measuring at least one reference blood sample from at least one subject having Alzheimer's Disease) which indicates that Alzheimer's Disease is absent in the patient.

A level which is “comparable with” the reference level in this respect means that the level is no more than 15%, preferably no more than 10%, more preferably no more than 5%, above the reference level or the level is no more than 15%, preferably no more than 10%, more preferably no more than 5%, below the reference level.

Alternatively, a level which is comparable with the reference level in this respect means that the detected level variation is within the accuracy of a measurement. The accuracy of a measurement depends on the measurement method used.

In particular, the level of the miRNA is at least 0.4-fold, at least 0.5-fold, at least 0.6-fold or at least 0.7-fold, preferably at least 0.8-fold or at least 0.9-fold, more preferably at least 1.2-fold or at least 1.5-fold, and even more preferably at least 2.0-fold or at least 3.0-fold below/above the reference level. For example, the level of the miRNA is at least 0.4-fold, at least 0.5-fold, at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

In one another embodiment, the determination of Alzheimer's Disease (AD) comprises determining whether the patient is at risk for developing AD. In this case, the reference level may be any level which allows to determine whether the patient is at risk for developing AD or not. It may be obtained from a (control) subject (i.e. a subject different from the patient to be tested) or from the same patient. In the latter case, the patient may be retested for AD. In particular, the reference level is the level determined by measuring at least one reference blood sample from at least one healthy subject (see above). This determination may have the form of a screening method. Said screening method allows an early detection of AD and, thus, the identification of patients having a predisposition or a likelihood (e.g. on the basis of their physical and/or physiological conditions) to develop AD, e.g. in the near future, such as within the next 1, 2, 3, 4, or 5 years.

Preferably,

the level of the miRNA having a nucleotide sequence selected from the group consisting of SEQ ID NO: 1, SEQ ID NO: 3, and SEQ ID NO: 18 is above the reference level (determined by measuring at least one reference blood sample from at least one healthy subject) which indicates that the patient is at risk for developing Alzheimer's Disease, and/or
the level of the miRNA having a nucleotide sequence selected from the group consisting of SEQ ID NO: 2 and SEQ ID NO: 4 to SEQ ID NO: 17 is below the reference level (determined by measuring at least one reference blood sample from at least one healthy subject) which indicates that the patient is at risk for developing Alzheimer's Disease.

In particular, the level of the miRNA is at least 0.4-fold, at least 0.5-fold, at least 0.6-fold or at least 0.7-fold, preferably at least 0.8-fold or at least 0.9-fold, more preferably at least 1.2-fold or at least 1.5-fold, and even more preferably at least 2.0-fold or at least 3.0-fold below/above the reference level. For example, the level of the miRNA is at least 0.4-fold, at least 0.5-fold, at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

In another embodiment, the determination of Alzheimer's Disease (AD) comprises determining the course of AD in the patient (having AD). In this case, the reference level may be any level which allows to determine the course of AD in the patient (having AD). It may be obtained from a (control) subject (i.e. a subject different from the patient to be tested/diagnosed) or from the same patient. In the latter case, the patient may be retested for AD. It may be determined whether AD worsens in the patient, whether AD does not worsen/is stable in the patient, or whether AD improves in the patient. In particular, the reference level is the level determined by measuring at least one reference blood sample from at least one subject having AD (see above).

Especially, said determining comprises determining the level (of each) of the at least three miRNAs comprised in the set in the blood sample at a first point in time and in at least one further blood sample at a later point in time and comparing said levels determined at the different time points.

Preferably,

the level of the miRNA having a nucleotide sequence selected from the group consisting SEQ ID NO: 1, SEQ ID NO: 3, and SEQ ID NO: 18 which
(i) increases over time indicates that Alzheimer's Disease worsens in the patient,
(ii) does not change over time indicates that Alzheimer's Disease does not worsen/is stable in the patient, or
(iii) decreases over time indicates that Alzheimer's Disease improves in the patient,
   and/or
   the level of the miRNA having a nucleotide sequence selected from the group consisting of SEQ ID NO: 2 and SEQ ID NO: 4 to SEQ ID NO: 17 which
(i) decreases over time indicates that Alzheimer's Disease worsens in the patient,
(ii) does not change over time indicates that Alzheimer's Disease does not worsen/is stable in the patient, or
(iii) increases over time indicates that Alzheimer's Disease improves in the patient.

As mentioned above, the detection of a decrease/an increase (dependent on the miRNA detected) of the level over time indicates that AD worsens in the patient. Preferably, said decrease/increase is at least 0.4-fold, at least 0.5-fold, at least 0.6-fold or at least 0.7-fold over time. More preferably, said decrease/increase is at least 0.8-fold or at least 0.9-fold over time. Even more preferably, said decrease/increase is at least 1.2-fold or at least 1.5-fold over time. Most preferably, said decrease/increase is at least 2.0-fold or at least 3.0-fold over time. For example, said decrease/increase may be determined over 1 year (12 months) or over 2 years (24 months).

As mentioned above, a level which does not change over time indicates that AD does not worsen/is stable in the patient. “Does not change over time” in this respect may mean that the level varies over time between 0 and <20%, e.g. 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 19.9, 19.99, or 19.999%. “Does not change over time” in this respect may also mean that the detected level variation is within the accuracy of a measurement. The accuracy of a measurement depends on the measurement method used. Preferably, the level is constant over time.

As mentioned above, the detection of an increase/a decrease (dependent on the miRNA detected) of the level over time indicates that AD improves in the patient. Preferably, said increase/decrease is at least 0.4-fold, at least 0.5-fold, at least 0.6-fold or at least 0.7-fold over time. More preferably, said increase/decrease is at least 0.8-fold or at least 0.9-fold over time. Even more preferably, said increase/decrease is at least 1.2-fold or at least 1.5-fold over time. Most preferably, said increase/decrease is at least 2.0-fold or at least 3.0-fold over time. For example, said increase/decrease may be determined over 1 year (12 months) or over 2 years (24 months).

The time period between the first point in time and the later point(s) in time preferably amounts to at least 1 day, at least 2 days, at least 3 days, at least 4 days, at least 5 days, at least 6 days, at least 7 days (1 week), at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months (1 year), at least 24 months (2 years), at least 3 years, at least 4 years, at least 5 years, at least 6 years, at least 7 years, at least 8 years, at least 9 years, or at least 10 years. For example, the patient may be routinely checked, e.g. once or twice a year. The patient may be (re)tested at 2, 3, 4, 5, 6 7, 8, 9, or 10 time points (first point in time and further point(s) in time).

In addition to the determination of the course of AD, the treatment of this disease can be monitored. It is namely preferred that the patient receives or has received a treatment, in particular therapeutic treatment, of AD during the determination of the course of AD. The treatment of AD may be selected from the group consisting of the administration of a drug, cognitive training, ergotherapy, and psychotherapy. The drug may be selected from the group consisting of antidementives, antidepressants, and neuroleptics.

The patient may receive a treatment during the complete determination/monitoring process (e.g. the administration of a drug) or may receive a treatment before, at, or after a first point in time (e.g. the administration of a drug) and may be retested at a later point in time. In particular, said first point in time may be before the initiation of a treatment and said later point in time may be during the treatment and/or after the treatment. If the treatment encompasses the administration of a drug and the patient responds to said treatment, the drug administration may be continued, the dose of the drug may be reduced, or the drug administration may be stopped. If the treatment encompasses the administration of a drug and the patient does not respond to said treatment, the dose of the drug may be increased, the drug may be changed, or the therapy mode may be changed, e.g. from drug administration to cognitive training, ergotherapy, and/or psychotherapy.

As mentioned above, it is preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for developing Alzheimer's Disease, and/or determining the course of Alzheimer's Disease in the patient. Thus, in view of the above, the method according to the second aspect of the present invention is preferably designated as a method

for diagnosing/determining whether a patient (suspected to be affected by AD) suffers from AD or not,
for determining the presence of AD in a patient (suspected to be affected by AD),
for determining the absence of AD in a patient (suspected to be affected by AD),
for determining the risk for developing AD in a patient and/or
for determining the course of AD in a patient (having/suffering from AD).

In a third aspect, the present invention relates to the (in vitro) use of at least three polynucleotides (probes or primers, in particular primer pairs) for detecting at least three miRNAs, e.g. at least 3, 4, 5, or 6 miRNAs, or 7 miRNAs, comprised in a set in a blood sample isolated from a patient for determining Alzheimer's Disease in the patient, wherein the first miRNA has a nucleotide sequence according to SEQ ID NO: 1 (hsa-miR-363-3p).

It is preferred that the second and third miRNAs have nucleotide sequences selected from the group consisting of SEQ ID NO: 2 to SEQ ID NO: 18.

It is more preferred that the second miRNA has a nucleotide sequence according to SEQ ID NO: 2 (hsa-miR-28-3p) or SEQ ID NO: 3 (hsa-let-7e-5p) and wherein the third miRNA has a nucleotide sequence selected from the group consisting of SEQ ID NO: 3 to SEQ ID NO: 18 under the proviso that the second and third miRNAs are different.

It is even more preferred that the second miRNA has a nucleotide sequence according to SEQ ID NO: 2 and the third miRNA has a nucleotide sequence selected from the group consisting of SEQ ID NO: 3 to SEQ ID NO: 15.

It is also even more preferred that the second miRNA has a nucleotide sequence according to SEQ ID NO: 3 and the third miRNA has a nucleotide sequence selected from the group consisting of SEQ ID NO: 2, SEQ ID NO: 4 to SEQ ID NO: 11, and SEQ ID NO: 16 to SEQ ID NO: 18.

It is particularly preferred that the second miRNA has a nucleotide sequence according to SEQ ID NO: 2 (hsa-miR-28-3p) and the third miRNA has a nucleotide sequence according to SEQ ID NO: 3 (hsa-let-7e-5p).

It is most preferred that the at least three miRNAs are comprised in a set selected from the group consisting of:
(a) SEQ ID NO: 1, SEQ ID NO: 2, and SEQ ID NO: 9,
(b) SEQ ID NO: 1, SEQ ID NO: 2, and SEQ ID NO: 4,
(c) SEQ ID NO: 1, SEQ ID NO: 2, and SEQ ID NO: 5,
(d) SEQ ID NO: 1, SEQ ID NO: 2, and SEQ ID NO: 6,
(e) SEQ ID NO: 1, SEQ ID NO: 2, SEQ ID NO: 6, and SEQ ID NO: 9,
(f) SEQ ID NO: 1, SEQ ID NO: 2, SEQ ID NO: 5, and SEQ ID NO: 12,
(g) SEQ ID NO: 1, SEQ ID NO: 2, SEQ ID NO: 3, SEQ ID NO: 6, SEQ ID NO: 8, and SEQ ID NO: 10,
(h) SEQ ID NO: 1, SEQ ID NO: 2, SEQ ID NO: 3, SEQ ID NO: 6, and SEQ ID NO: 8,
(i) SEQ ID NO: 1, SEQ ID NO: 2, SEQ ID NO: 5, SEQ ID NO: 10, SEQ ID NO: 12, and SEQ ID NO: 13,
(j) SEQ ID NO: 1, SEQ ID NO: 2, SEQ ID NO: 3, SEQ ID NO: 5, SEQ ID NO: 6, and SEQ ID NO: 11,
(k) SEQ ID NO: 1, SEQ ID NO: 2, SEQ ID NO: 3, SEQ ID NO: 4, SEQ ID NO: 7, SEQ ID NO: 8, and SEQ ID NO: 11,
(l) SEQ ID NO: 1, SEQ ID NO: 2, SEQ ID NO: 12, and SEQ ID NO: 13,
(m) SEQ ID NO: 1, SEQ ID NO: 2, SEQ ID NO: 5, SEQ ID NO: 9, and SEQ ID NO: 12,
(n) SEQ ID NO: 1, SEQ ID NO: 2, SEQ ID NO: 7, SEQ ID NO: 9, and SEQ ID NO: 12,
(o) SEQ ID NO: 1, SEQ ID NO: 2, SEQ ID NO: 9, SEQ ID NO: 10, and SEQ ID NO: 12,
(p) SEQ ID NO: 1, SEQ ID NO: 2, and SEQ ID NO: 12,
(q) SEQ ID NO: 1, SEQ ID NO: 2, and SEQ ID NO: 14,
(r) SEQ ID NO: 1, SEQ ID NO: 2, SEQ ID NO: 9, and SEQ ID NO: 12,
(s) SEQ ID NO: 1, SEQ ID NO: 2, and SEQ ID NO: 15,
(t) SEQ ID NO: 1, SEQ ID NO: 3, SEQ ID NO: 4, SEQ ID NO: 7, and SEQ ID NO: 11,
(u) SEQ ID NO: 1, SEQ ID NO: 3, SEQ ID NO: 5, SEQ ID NO: 8, SEQ ID NO: 9, and SEQ ID NO: 10,
(v) SEQ ID NO: 1, SEQ ID NO: 3, SEQ ID NO: 4, SEQ ID NO: 5, SEQ ID NO: 7, SEQ ID NO: 11, and SEQ ID NO: 16,
(w) SEQ ID NO: 1, SEQ ID NO: 3, SEQ ID NO: 5, SEQ ID NO: 6, SEQ ID NO: 8, SEQ ID NO: 16, and SEQ ID NO: 17,
(x) SEQ ID NO: 1, SEQ ID NO: 3, SEQ ID NO: 5, SEQ ID NO: 8, SEQ ID NO: 16, SEQ ID NO: 17, and SEQ ID NO: 18, and
(y) SEQ ID NO: 1, SEQ ID NO: 3, SEQ ID NO: 6, SEQ ID NO: 7, SEQ ID NO: 8, SEQ ID NO: 16, and SEQ ID NO: 17.

It should be noted that the at least three miRNAs have nucleotide sequences as shown in the sets according to (a) to (y). For example the set according to (a) comprises the miRNA having a nucleotide sequence according to SEQ ID NO: 1, the miRNA having a nucleotide sequence according to SEQ ID NO: 2, and the miRNA having a nucleotide sequence according to SEQ ID NO: 9.

The patient who's miRNAs are detected may be a patient suspected of suffering from AD or a patient suffering from AD.

The at least three polynucleotides may be probes or primers, in particular primer pairs. For example, three polynucleotide probes are used for detecting three miRNAs comprised in a set in a blood sample isolated from a patient for determining Alzheimer's Disease in the patient or three primer pairs (polynucleotide pairs) are used for detecting three miRNAs comprised in a set in a blood sample isolated from a patient for determining Alzheimer's Disease in the patient.

Preferably,
(i) the polynucleotides are at least partially (reverse) complementary, preferably (reverse) complementary, to the (respective) miRNAs mentioned above, or
(ii) the polynucleotides have at least 90%, preferably at least 95%, more preferably at least 99%, i.e. at least 90, 91, 92, 93, 94, 95, 96, 97, 98, or 99%, sequence identity to the polynucleotides according to (i).

It is particularly preferred that the polynucleotides as defined in (ii) have at least 90%, preferably at least 95%, more preferably at least 99%, i.e. at least 90, 91, 92, 93, 94, 95, 96, 97, 98, or 99%, sequence identity over a continuous stretch of at least 10, 11, 12, 13, 14, 15, 16, 17, 18, 19, 20, 21, 22, 23, 24, 25, or more nucleotides, preferably over the whole length, to the polynucleotides according to (i).

In addition, the polynucleotides as defined in (ii) (i.e. polynucleotide variants) are only regarded as polynucleotides as defined in (ii) (i.e. polynucleotide variants) within the context of the present invention, if they are still capable of binding to, hybridizing with, or detecting the respective target nucleic acid molecules, i.e. the target nucleic acid molecules comprising a nucleotide sequence selected from the group consisting of SEQ ID NO: 1 to SEQ ID NO: 18, through one or more types of chemical bonds, usually through complementary base pairing, usually through hydrogen bond formation under stringent hybridization conditions. The skilled person can readily assess whether the polynucleotides as defined in (ii) (i.e. polynucleotide variants) are still capable of binding to, hybridizing with, recognizing or detecting the respective target nucleic acid molecules, i.e. the target nucleic acid molecules comprising a nucleotide sequence selected from the group consisting of SEQ ID NO: 1 to SEQ ID NO: 18. Suitable assays to determine whether hybridization under stringent conditions still occurs are well known in the art. However, as an example, a suitable assay to determine whether hybridization still occurs comprises the steps of: (a) incubating the polynucleotides as defined in (ii) attached onto a biochip with the respective target nucleic acid molecules, i.e. the target nucleic acid molecules comprising a nucleotide sequence selected from the group consisting of SEQ ID NO: 1 to SEQ ID NO: 18, (b) washing the biochip to remove unspecific bindings, (c) subjecting the biochip to a detection system, and (d) analyzing whether the polynucleotides can still hybridize with the respective target nucleic acid molecules. As a positive control, the respective non-mutated polynucleotides as defined in (i) may be used. Preferable stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.; or 6×SSPE, 10% formamide, 0.01%, Tween 20, 0.1×TE buffer, 0.5 mg/ml BSA, 0.1 mg/ml herring sperm DNA, incubating at 42° C. with wash in 05×SSPE and 6×SSPE at 45° C. The same applies to variants of miRNAs having a nucleotide sequence selected from the group consisting of SEQ ID NO: 19 to SEQ ID NO: 24.

In one preferred embodiment, the primers for detecting a miRNA comprised in a set in a blood sample isolated from a patient for determining Alzheimer's Disease in the patient encompass:
(i) a miRNA-specific primer for reverse transcription of miRNA (comprised in a total RNA fraction isolated from a blood sample, preferably comprised in a total RNA fraction isolated from a whole blood or a blood cell fraction) in miRNA-specific cDNA, e.g. a stem-loop reverse primer having a nucleotide sequence according to SEQ ID NO: 25 to SEQ ID NO: 35, and/or
(ii) a primer set comprising a forward primer which is specific for the cDNA obtained from the miRNA and an universal reverse primer for amplifying the cDNA obtained from the miRNA via real time polymerase chain reaction (RT-PCR) such as real time quantitative polymerase chain reaction (RT-qPCR), e.g. a primer set comprising a forward primer having a nucleotide sequence according to SEQ ID NO: 36 to SEQ ID NO: 46 and an universal reverse primer having a nucleotide sequence according to SEQ ID NO: 47.

Thus, the primers for detecting

the miRNA having a nucleotide sequence according to SEQ ID NO: 1 preferably encompass the stem-loop reverse primer having a nucleotide sequence according to SEQ ID NO: 25 and/or the forward primer having a nucleotide sequence according to SEQ ID NO: 36 and the universal reverse primer having a nucleotide sequence according to SEQ ID NO: 47,
the miRNA having a nucleotide sequence according to SEQ ID NO: 2 preferably encompass the stem-loop reverse primer having a nucleotide sequence according to SEQ ID NO: 26 and/or the forward primer having a nucleotide sequence according to SEQ ID NO: 37 and the universal reverse primer having a nucleotide sequence according to SEQ ID NO: 47,
the miRNA having a nucleotide sequence according to SEQ ID NO: 3 preferably encompass the stem-loop reverse primer having a nucleotide sequence according to SEQ ID NO: 27 and/or the forward primer having a nucleotide sequence according to SEQ ID NO: 38 and the universal reverse primer having a nucleotide sequence according to SEQ ID NO: 47,
the miRNA having a nucleotide sequence according to SEQ ID NO: 4 preferably encompass the stem-loop reverse primer having a nucleotide sequence according to SEQ ID NO: 28 and/or the forward primer having a nucleotide sequence according to SEQ ID NO: 39 and the universal reverse primer having a nucleotide sequence according to SEQ ID NO: 47,
the miRNA having a nucleotide sequence according to SEQ ID NO: 5 preferably encompass the stem-loop reverse primer having a nucleotide sequence according to SEQ ID NO: 29 and/or the forward primer having a nucleotide sequence according to SEQ ID NO: 40 and the universal reverse primer having a nucleotide sequence according to SEQ ID NO: 47,
the miRNA having a nucleotide sequence according to SEQ ID NO: 6 preferably encompass the stem-loop reverse primer having a nucleotide sequence according to SEQ ID NO: 30 and/or the forward primer having a nucleotide sequence according to SEQ ID NO: 41 and the universal reverse primer having a nucleotide sequence according to SEQ ID NO: 47,
the miRNA having a nucleotide sequence according to SEQ ID NO: 9 preferably encompass the stem-loop reverse primer having a nucleotide sequence according to SEQ ID NO: 31 and/or the forward primer having a nucleotide sequence according to SEQ ID NO: 42 and the universal reverse primer having a nucleotide sequence according to SEQ ID NO: 47,
the miRNA having a nucleotide sequence according to SEQ ID NO: 10 preferably encompass the stem-loop reverse primer having a nucleotide sequence according to SEQ ID NO: 32 and/or the forward primer having a nucleotide sequence according to SEQ ID NO: 43 and the universal reverse primer having a nucleotide sequence according to SEQ ID NO: 47,
the miRNA having a nucleotide sequence according to SEQ ID NO: 12 preferably encompass the stem-loop reverse primer having a nucleotide sequence according to SEQ ID NO: 33 and/or the forward primer having a nucleotide sequence according to SEQ ID NO: 44 and the universal reverse primer having a nucleotide sequence according to SEQ ID NO: 47,
the miRNA having a nucleotide sequence according to SEQ ID NO: 14 preferably encompass the stem-loop reverse primer having a nucleotide sequence according to SEQ ID NO: 34 and/or the forward primer having a nucleotide sequence according to SEQ ID NO: 45 and the universal reverse primer having a nucleotide sequence according to SEQ ID NO: 47, and/or
the miRNA having a nucleotide sequence according to SEQ ID NO: 15 preferably encompass the stem-loop reverse primer having a nucleotide sequence according to SEQ ID NO: 35 and/or the forward primer having a nucleotide sequence according to SEQ ID NO: 46 and the universal reverse primer having a nucleotide sequence according to SEQ ID NO: 47.

In a still further embodiment, the primers for detecting the at least three
a) miRNAs with SEQ ID NO 1, 2 and 9 (Set-No. SA-1) encompass stem-loop reverse primers having a nucleotide sequence according to SEQ ID NO: 25, 26, 31 and forward primers having a nucleotide sequence according to SEQ ID NO: 36, 37, 42 and the universal reverse primer having a nucleotide sequence according to SEQ ID NO: 47,
b) miRNAs with SEQ ID NO 1, 2 and 4 (Set-No. SA-2) encompass stem-loop reverse primers having a nucleotide sequence according to SEQ ID NO: 25, 26, 28 and forward primers having a nucleotide sequence according to SEQ ID NO: 36, 37, 39 and the universal reverse primer having a nucleotide sequence according to SEQ ID NO: 47,
c) miRNAs with SEQ ID NO 1, 2 and 5 (Set-No. SA-3) encompass stem-loop reverse primers having a nucleotide sequence according to SEQ ID NO: 25, 26, 29 and forward primers having a nucleotide sequence according to SEQ ID NO: 36, 37, 40 and the universal reverse primer having a nucleotide sequence according to SEQ ID NO: 47,
d) miRNAs with SEQ ID NO 1, 2 and 6 (Set-No. SA-4) encompass stem-loop reverse primers having a nucleotide sequence according to SEQ ID NO: 25, 26, 30 and forward primers having a nucleotide sequence according to SEQ ID NO: 36, 37, 41 and the universal reverse primer having a nucleotide sequence according to SEQ ID NO: 47,
e) miRNAs with SEQ ID NO 1, 2 and 12 (Set-No. SA-16) encompass stem-loop reverse primers having a nucleotide sequence according to SEQ ID NO: 25, 26, 33 and forward primers having a nucleotide sequence according to SEQ ID NO: 36, 37, 44 and the universal reverse primer having a nucleotide sequence according to SEQ ID NO: 47,
f) miRNAs with SEQ ID NO 1, 2, 6 and 9 (Set-No. SA-5) encompass stem-loop reverse primers having a nucleotide sequence according to SEQ ID NO: 25, 26, 30, 31 and forward primers having a nucleotide sequence according to SEQ ID NO: 36, 37, 41, 42 and the universal reverse primer having a nucleotide sequence according to SEQ ID NO: 47,
g) miRNAs with SEQ ID NO 1, 2, 5 and 12 (Set-No. SA-6) encompass stem-loop reverse primers having a nucleotide sequence according to SEQ ID NO: 25, 26, 29, 33 and forward primers having a nucleotide sequence according to SEQ ID NO: 36, 37, 40, 44 and the universal reverse primer having a nucleotide sequence according to SEQ ID NO: 47,
h) miRNAs with SEQ ID NO 1, 2, 5, 9 and 12 (Set-No. SA-13) encompass stem-loop reverse primers having a nucleotide sequence according to SEQ ID NO: 25, 26, 29, 31, 33 and forward primers having a nucleotide sequence according to SEQ ID NO: 36, 37, 40, 42, 44 and the universal reverse primer having a nucleotide sequence according to SEQ ID NO: 47, and/or
i) miRNAs with SEQ ID NO 1, 2, 9, 10 and 12 (Set-No. SA-15) encompass stem-loop reverse primers having a nucleotide sequence according to SEQ ID NO: 25, 26, 31, 32, 33 and forward primers having a nucleotide sequence according to SEQ ID NO: 36, 37, 42, 43, 44 and the universal reverse primer having a nucleotide sequence according to SEQ ID NO: 47.

The polynucleotides (probes or primers, in particular primer pair) described above are useful for conducting the method according to the first aspect of the present invention.

It is particularly preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for developing Alzheimer's Disease, and/or determining the course of Alzheimer's Disease in the patient. Thus, in view of the above, the use according to the third aspect of the present invention is preferably designated as a use

for diagnosing/determining whether a patient (suspected to be affected by AD) suffers from AD or not,
for determining the presence of AD in a patient (suspected to be affected by AD),
for determining the absence of AD in a patient (suspected to be affected by AD),
for determining the risk for developing AD in a patient, and/or
for determining the course of AD in a patient (having/suffering from AD).

In a fourth aspect, the present invention relates to the (in vitro) use of at least three polynucleotides (probes or primers, in particular primer pairs) for detecting at least three miRNAs, e.g. at least 3, 4, 5, or 6 miRNAs, or 7 miRNAs, in a blood sample isolated from a patient for determining Alzheimer's Disease in the patient, wherein the at least three miRNAs are comprised in a set selected from the group consisting of:
(a) SEQ ID NO: 1, SEQ ID NO: 2, and SEQ ID NO: 9,
(b) SEQ ID NO: 1, SEQ ID NO: 2, and SEQ ID NO: 4,
(c) SEQ ID NO: 1, SEQ ID NO: 2, and SEQ ID NO: 5,
(d) SEQ ID NO: 1, SEQ ID NO: 2, and SEQ ID NO: 6,
(e) SEQ ID NO: 1, SEQ ID NO: 2, SEQ ID NO: 6, and SEQ ID NO: 9,
(f) SEQ ID NO: 1, SEQ ID NO: 2, SEQ ID NO: 5, and SEQ ID NO: 12,
(g) SEQ ID NO: 1, SEQ ID NO: 2, SEQ ID NO: 3, SEQ ID NO: 6, SEQ ID NO: 8, and SEQ ID NO: 10,
(h) SEQ ID NO: 1, SEQ ID NO: 2, SEQ ID NO: 3, SEQ ID NO: 6, and SEQ ID NO: 8,
(i) SEQ ID NO: 1, SEQ ID NO: 2, SEQ ID NO: 5, SEQ ID NO: 10, SEQ ID NO: 12, and SEQ ID NO: 13,
(j) SEQ ID NO: 1, SEQ ID NO: 2, SEQ ID NO: 3, SEQ ID NO: 5, SEQ ID NO: 6, and SEQ ID NO: 11,
(k) SEQ ID NO: 1, SEQ ID NO: 2, SEQ ID NO: 3, SEQ ID NO: 4, SEQ ID NO: 7, SEQ ID NO: 8, and SEQ ID NO: 11,
(l) SEQ ID NO: 1, SEQ ID NO: 2, SEQ ID NO: 12, and SEQ ID NO: 13,
(m) SEQ ID NO: 1, SEQ ID NO: 2, SEQ ID NO: 5, SEQ ID NO: 9, and SEQ ID NO: 12,
(n) SEQ ID NO: 1, SEQ ID NO: 2, SEQ ID NO: 7, SEQ ID NO: 9, and SEQ ID NO: 12,
(o) SEQ ID NO: 1, SEQ ID NO: 2, SEQ ID NO: 9, SEQ ID NO: 10, and SEQ ID NO: 12,
(p) SEQ ID NO: 1, SEQ ID NO: 2, and SEQ ID NO: 12,
(q) SEQ ID NO: 1, SEQ ID NO: 2, and SEQ ID NO: 14,
(r) SEQ ID NO: 1, SEQ ID NO: 2, SEQ ID NO: 9, and SEQ ID NO: 12,
(s) SEQ ID NO: 1, SEQ ID NO: 2, and SEQ ID NO: 15,
(t) SEQ ID NO: 1, SEQ ID NO: 3, SEQ ID NO: 4, SEQ ID NO: 7, and SEQ ID NO: 11,
(u) SEQ ID NO: 1, SEQ ID NO: 3, SEQ ID NO: 5, SEQ ID NO: 8, SEQ ID NO: 9, and SEQ ID NO: 10,
(v) SEQ ID NO: 1, SEQ ID NO: 3, SEQ ID NO: 4, SEQ ID NO: 5, SEQ ID NO: 7, SEQ ID NO: 11, and SEQ ID NO: 16,
(w) SEQ ID NO: 1, SEQ ID NO: 3, SEQ ID NO: 5, SEQ ID NO: 6, SEQ ID NO: 8, SEQ ID NO: 16, and SEQ ID NO: 17,
(x) SEQ ID NO: 1, SEQ ID NO: 3, SEQ ID NO: 5, SEQ ID NO: 8, SEQ ID NO: 16, SEQ ID NO: 17, and SEQ ID NO: 18, and
(y) SEQ ID NO: 1, SEQ ID NO: 3, SEQ ID NO: 6, SEQ ID NO: 7, SEQ ID NO: 8, SEQ ID NO: 16, and SEQ ID NO: 17.

It should be noted that the at least three miRNAs have nucleotide sequences as shown in the sets according to (a) to (y). For example the set according to (a) comprises the miRNA having a nucleotide sequence according to SEQ ID NO: 1, the miRNA having a nucleotide sequence according to SEQ ID NO: 2, and the miRNA having a nucleotide sequence according to SEQ ID NO: 9.

The patient who's miRNAs are detected may be a patient suspected of suffering from AD or a patient suffering from AD.

The at least three polynucleotides may be probes or primers, in particular primer pairs.

Preferably,
(i) the polynucleotides are at least partially (reverse) complementary, preferably (reverse) complementary, to the (respective) miRNAs mentioned above, or
(ii) the polynucleotides have at least 90%, preferably at least 95%, more preferably at least 99%, i.e. at least 90, 91, 92, 93, 94, 95, 96, 97, 98, or 99%, sequence identity to the polynucleotides according to (i).

It is particularly preferred that the polynucleotides as defined in (ii) have at least 90%, preferably at least 95%, more preferably at least 99%, i.e. at least 90, 91, 92, 93, 94, 95, 96, 97, 98, or 99%, sequence identity over a continuous stretch of at least 10, 11, 12, 13, 14, 15, 16, 17, 18, 19, 20, 21, 22, 23, 24, 25, or more nucleotides, preferably over the whole length, to the polynucleotides according to (i).

As to the polynucleotide variants, it is referred to the third aspect of the present invention.

As to the specific primers, in particular primer pairs, e.g. the primers having a nucleotide sequence according to SEQ ID NO: 25 to SEQ ID NO: 47, for detecting the miRNAs comprised in a set in a blood sample isolated from a patient for determining Alzheimer's Disease in the patient, it is referred to the third aspect of the present invention.

The polynucleotides (probes or primers, in particular primer pair) described above are useful for conducting the method according to the second aspect of the present invention.

It is particularly preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for developing Alzheimer's Disease, and/or determining the course of Alzheimer's Disease in the patient. Thus, in view of the above, the use according to the fourth aspect of the present invention is preferably designated as a use

for diagnosing/determining whether a patient (suspected to be affected by AD) suffers from AD or not,
for determining the presence of AD in a patient (suspected to be affected by AD),
for determining the absence of AD in a patient (suspected to be affected by AD),
for determining the risk for developing AD in a patient, and/or
for determining the course of AD in a patient (having/suffering from AD).

In a fifth aspect, the present invention relates to (the use of) a kit for determining Alzheimer's Disease in a patient which comprises:
(i) means for determining the level (of each) of at least three miRNAs, e.g. at least 3, 4, 5, or 6 miRNAs, or 7 miRNAs, comprised in a set in a blood sample isolated from a patient, wherein the first miRNA has a nucleotide sequence according to SEQ ID NO: 1 (hsa-miR-363-3p) or a nucleotide sequence having at least 90%, preferably at least 95%, more preferably at least 99%, i.e. at least 90, 91, 92, 93, 94, 95, 96, 97, 98, or 99%, sequence identity thereto, and
(ii) optionally at least three references.

It is preferred that the second and third miRNAs have nucleotide sequences selected from the group consisting of SEQ ID NO: 2 to SEQ ID NO: 18. Alternatively, the nucleotide sequences of the miRNAs according to SEQ ID NO: 2 to SEQ ID NO: 18 are nucleotide sequences having at least 90%, preferably at least 95%, more preferably at least 99%, i.e. at least 90, 91, 92, 93, 94, 95, 96, 97, 98, or 99%, sequence identity thereto.

It is more preferred that the second miRNA has a nucleotide sequence according to SEQ ID NO: 2 (hsa-miR-28-3p) or SEQ ID NO: 3 (hsa-let-7e-5p) and wherein the third miRNA has a nucleotide sequence selected from the group consisting of SEQ ID NO: 3 to SEQ ID NO: 18 under the proviso that the second and third miRNAs are different.

It is even more preferred that the second miRNA has a nucleotide sequence according to SEQ ID NO: 2 and the third miRNA has a nucleotide sequence selected from the group consisting of SEQ ID NO: 3 to SEQ ID NO: 15.

It is also even more preferred that the second miRNA has a nucleotide sequence according to SEQ ID NO: 3 and the third miRNA has a nucleotide sequence selected from the group consisting of SEQ ID NO: 2, SEQ ID NO: 4 to SEQ ID NO: 11, and SEQ ID NO: 16 to SEQ ID NO: 18.

It is particularly preferred that the second miRNA has a nucleotide sequence according to SEQ ID NO: 2 (hsa-miR-28-3p) and the third miRNA has a nucleotide sequence according to SEQ ID NO: 3 (hsa-let-7e-5p).

It is most preferred that the at least three miRNAs are comprised in a set selected from the group consisting of:
(a) SEQ ID NO: 1, SEQ ID NO: 2, and SEQ ID NO: 9,
(b) SEQ ID NO: 1, SEQ ID NO: 2, and SEQ ID NO: 4,
(c) SEQ ID NO: 1, SEQ ID NO: 2, and SEQ ID NO: 5,
(d) SEQ ID NO: 1, SEQ ID NO: 2, and SEQ ID NO: 6,
(e) SEQ ID NO: 1, SEQ ID NO: 2, SEQ ID NO: 6, and SEQ ID NO: 9,
(f) SEQ ID NO: 1, SEQ ID NO: 2, SEQ ID NO: 5, and SEQ ID NO: 12,
(g) SEQ ID NO: 1, SEQ ID NO: 2, SEQ ID NO: 3, SEQ ID NO: 6, SEQ ID NO: 8, and SEQ ID NO: 10,
(h) SEQ ID NO: 1, SEQ ID NO: 2, SEQ ID NO: 3, SEQ ID NO: 6, and SEQ ID NO: 8,
(i) SEQ ID NO: 1, SEQ ID NO: 2, SEQ ID NO: 5, SEQ ID NO: 10, SEQ ID NO: 12, and SEQ ID NO: 13,
(j) SEQ ID NO: 1, SEQ ID NO: 2, SEQ ID NO: 3, SEQ ID NO: 5, SEQ ID NO: 6, and SEQ ID NO: 11,
(k) SEQ ID NO: 1, SEQ ID NO: 2, SEQ ID NO: 3, SEQ ID NO: 4, SEQ ID NO: 7, SEQ ID NO: 8, and SEQ ID NO: 11,
(l) SEQ ID NO: 1, SEQ ID NO: 2, SEQ ID NO: 12, and SEQ ID NO: 13,
(m) SEQ ID NO: 1, SEQ ID NO: 2, SEQ ID NO: 5, SEQ ID NO: 9, and SEQ ID NO: 12,
(n) SEQ ID NO: 1, SEQ ID NO: 2, SEQ ID NO: 7, SEQ ID NO: 9, and SEQ ID NO: 12,
(o) SEQ ID NO: 1, SEQ ID NO: 2, SEQ ID NO: 9, SEQ ID NO: 10, and SEQ ID NO: 12,
(p) SEQ ID NO: 1, SEQ ID NO: 2, and SEQ ID NO: 12,
(q) SEQ ID NO: 1, SEQ ID NO: 2, and SEQ ID NO: 14,
(r) SEQ ID NO: 1, SEQ ID NO: 2, SEQ ID NO: 9, and SEQ ID NO: 12,
(s) SEQ ID NO: 1, SEQ ID NO: 2, and SEQ ID NO: 15,
(t) SEQ ID NO: 1, SEQ ID NO: 3, SEQ ID NO: 4, SEQ ID NO: 7, and SEQ ID NO: 11,
(u) SEQ ID NO: 1, SEQ ID NO: 3, SEQ ID NO: 5, SEQ ID NO: 8, SEQ ID NO: 9, and SEQ ID NO: 10,
(v) SEQ ID NO: 1, SEQ ID NO: 3, SEQ ID NO: 4, SEQ ID NO: 5, SEQ ID NO: 7, SEQ ID NO: 11, and SEQ ID NO: 16,
(w) SEQ ID NO: 1, SEQ ID NO: 3, SEQ ID NO: 5, SEQ ID NO: 6, SEQ ID NO: 8, SEQ ID NO: 16, and SEQ ID NO: 17,
(x) SEQ ID NO: 1, SEQ ID NO: 3, SEQ ID NO: 5, SEQ ID NO: 8, SEQ ID NO: 16, SEQ ID NO: 17, and SEQ ID NO: 18, and
(y) SEQ ID NO: 1, SEQ ID NO: 3, SEQ ID NO: 6, SEQ ID NO: 7, SEQ ID NO: 8, SEQ ID NO: 16, and SEQ ID NO: 17.

It should be noted that the at least three miRNAs have nucleotide sequences as shown in the sets according to (a) to (y). For example the set according to (a) comprises the miRNA having a nucleotide sequence according to SEQ ID NO: 1, the miRNA having a nucleotide sequence according to SEQ ID NO: 2, and the miRNA having a nucleotide sequence according to SEQ ID NO: 9.

The patient who's miRNA level can be determined may be a patient suspected of suffering from AD or a patient suffering from AD.

Especially, the kit is useful for conducting the method according to the first aspect of the present invention.

It is particularly preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for developing Alzheimer's Disease, and/or determining the course of Alzheimer's Disease in the patient. Thus, in view of the above, the kit according to the fifth aspect of the present invention is preferably designated as a kit

for diagnosing/determining whether a patient (suspected to be affected by AD) suffers from AD or not,
for determining the presence of AD in a patient (suspected to be affected by AD),
for determining the absence of AD in a patient (suspected to be affected by AD),
for determining the risk for developing AD in a patient, and/or
for determining the course of AD in a patient (having/suffering from AD).

The references may be any references which allow to determine AD in the patient. In particular, the references may be any references which allows to diagnose whether a patient (suspected of having AD) suffers from AD or not, to determine whether AD is present in the patient (suspected of having AD), to determine whether AD is absent in the patient (suspected of having AD), to determine whether the patient has a predisposition to develop AD or will likely develop AD (in the (near) future), and/or to determine the course of AD in the patient (having AD). In this respect, it is also referred to the preferred embodiments mentioned in the context of the first aspect of the present invention.

In particular, the means for determining the level (of each) of the at least three miRNAs comprise:

at least three polynucleotides (polynucleotide probes) (for each miRNA to be detected a specific polynucleotide probe), in particular according to the third aspect of the present invention,
at least three primer pairs (for each miRNA to be detected a specific primer pair), and/or
at least three polynucleotides (polynucleotide probes), in particular according to the third aspect of the present invention, and at least three antibodies capable of binding a hybrid of said polynucleotides (polynucleotide probes) and said miRNAs (for each miRNA to be detected a specific polynucleotide probe and an antibody capable of binding a hybrid of said polynucleotide probe and said miRNA).

In one preferred embodiment, the means for determining the level of a (specific) miRNA comprised in a set in a blood sample isolated from a patient encompass:
(i) a miRNA-specific primer for reverse transcription of miRNA (comprised in a total RNA fraction isolated from a blood sample, preferably comprised in a total RNA fraction isolated from a whole blood or a blood cell fraction) in miRNA-specific cDNA, e.g. a stem-loop reverse primer having a nucleotide sequence according to SEQ ID NO: 25 to SEQ ID NO: 35, and/or
(ii) a primer set comprising a forward primer which is specific for the cDNA obtained from the miRNA and an universal reverse primer for amplifying the cDNA obtained from the miRNA via real time polymerase chain reaction (RT-PCR) such as real time quantitative polymerase chain reaction (RT-qPCR), e.g. a primer set comprising a forward primer having a nucleotide sequence according to SEQ ID NO: 36 to SEQ ID NO: 46 and an universal reverse primer having a nucleotide sequence according to SEQ ID NO: 47.

Thus, the primers for detecting

the miRNA having a nucleotide sequence according to SEQ ID NO: 1 preferably encompass the stem-loop reverse primer having a nucleotide sequence according to SEQ ID NO: 25 and/or the forward primer having a nucleotide sequence according to SEQ ID NO: 36 and the universal reverse primer having a nucleotide sequence according to SEQ ID NO: 47,
the miRNA having a nucleotide sequence according to SEQ ID NO: 2 preferably encompass the stem-loop reverse primer having a nucleotide sequence according to SEQ ID NO: 26 and/or the forward primer having a nucleotide sequence according to SEQ ID NO: 37 and the universal reverse primer having a nucleotide sequence according to SEQ ID NO: 47,
the miRNA having a nucleotide sequence according to SEQ ID NO: 3 preferably encompass the stem-loop reverse primer having a nucleotide sequence according to SEQ ID NO: 27 and/or the forward primer having a nucleotide sequence according to SEQ ID NO: 38 and the universal reverse primer having a nucleotide sequence according to SEQ ID NO: 47,
the miRNA having a nucleotide sequence according to SEQ ID NO: 4 preferably encompass the stem-loop reverse primer having a nucleotide sequence according to SEQ ID NO: 28 and/or the forward primer having a nucleotide sequence according to SEQ ID NO: 39 and the universal reverse primer having a nucleotide sequence according to SEQ ID NO: 47,
the miRNA having a nucleotide sequence according to SEQ ID NO: 5 preferably encompass the stem-loop reverse primer having a nucleotide sequence according to SEQ ID NO: 29 and/or the forward primer having a nucleotide sequence according to SEQ ID NO: 40 and the universal reverse primer having a nucleotide sequence according to SEQ ID NO: 47,
the miRNA having a nucleotide sequence according to SEQ ID NO: 6 preferably encompass the stem-loop reverse primer having a nucleotide sequence according to SEQ ID NO: 30 and/or the forward primer having a nucleotide sequence according to SEQ ID NO: 41 and the universal reverse primer having a nucleotide sequence according to SEQ ID NO: 47,
the miRNA having a nucleotide sequence according to SEQ ID NO: 9 preferably encompass the stem-loop reverse primer having a nucleotide sequence according to SEQ ID NO: 31 and/or the forward primer having a nucleotide sequence according to SEQ ID NO: 42 and the universal reverse primer having a nucleotide sequence according to SEQ ID NO: 47,
the miRNA having a nucleotide sequence according to SEQ ID NO: 10 preferably encompass the stem-loop reverse primer having a nucleotide sequence according to SEQ ID NO: 32 and/or the forward primer having a nucleotide sequence according to SEQ ID NO: 43 and the universal reverse primer having a nucleotide sequence according to SEQ ID NO: 47,
the miRNA having a nucleotide sequence according to SEQ ID NO: 12 preferably encompass the stem-loop reverse primer having a nucleotide sequence according to SEQ ID NO: 33 and/or the forward primer having a nucleotide sequence according to SEQ ID NO: 44 and the universal reverse primer having a nucleotide sequence according to SEQ ID NO: 47,
the miRNA having a nucleotide sequence according to SEQ ID NO: 14 preferably encompass the stem-loop reverse primer having a nucleotide sequence according to SEQ ID NO: 34 and/or the forward primer having a nucleotide sequence according to SEQ ID NO: 45 and the universal reverse primer having a nucleotide sequence according to SEQ ID NO: 47, and/or
the miRNA having a nucleotide sequence according to SEQ ID NO: 15 preferably encompass the stem-loop reverse primer having a nucleotide sequence according to SEQ ID NO: 35 and/or
the forward primer having a nucleotide sequence according to SEQ ID NO: 46 and the universal reverse primer having a nucleotide sequence according to SEQ ID NO: 47.

Additionally or alternatively, the kit preferably comprises (a) dual-labelled probe(s) having a nucleotide sequence selected from the group consisting of SEQ ID NO: 48 to SEQ ID NO: 58.

In a still further embodiment, the means comprise primers for detecting the level of the at least three
a) miRNAs with SEQ ID NO 1, 2 and 9 (Set-No. SA-1) encompass stem-loop reverse primers having a nucleotide sequence according to SEQ ID NO: 25, 26, 31 and forward primers having a nucleotide sequence according to SEQ ID NO: 36, 37, 42 and the universal reverse primer having a nucleotide sequence according to SEQ ID NO: 47,
b) miRNAs with SEQ ID NO 1, 2 and 4 (Set-No. SA-2) encompass stem-loop reverse primers having a nucleotide sequence according to SEQ ID NO: 25, 26, 28 and forward primers having a nucleotide sequence according to SEQ ID NO: 36, 37, 39 and the universal reverse primer having a nucleotide sequence according to SEQ ID NO: 47,
c) miRNAs with SEQ ID NO 1, 2 and 5 (Set-No. SA-3) encompass stem-loop reverse primers having a nucleotide sequence according to SEQ ID NO: 25, 26, 29 and forward primers having a nucleotide sequence according to SEQ ID NO: 36, 37, 40 and the universal reverse primer having a nucleotide sequence according to SEQ ID NO: 47,
d) miRNAs with SEQ ID NO 1, 2 and 6 (Set-No. SA-4) encompass stem-loop reverse primers having a nucleotide sequence according to SEQ ID NO: 25, 26, 30 and forward primers having a nucleotide sequence according to SEQ ID NO: 36, 37, 41 and the universal reverse primer having a nucleotide sequence according to SEQ ID NO: 47,
e) miRNAs with SEQ ID NO 1, 2 and 12 (Set-No. SA-16) encompass stem-loop reverse primers having a nucleotide sequence according to SEQ ID NO: 25, 26, 33 and forward primers having a nucleotide sequence according to SEQ ID NO: 36, 37, 44 and the universal reverse primer having a nucleotide sequence according to SEQ ID NO: 47,
f) miRNAs with SEQ ID NO 1, 2, 6 and 9 (Set-No. SA-5) encompass stem-loop reverse primers having a nucleotide sequence according to SEQ ID NO: 25, 26, 30, 31 and forward primers having a nucleotide sequence according to SEQ ID NO: 36, 37, 41, 42 and the universal reverse primer having a nucleotide sequence according to SEQ ID NO: 47,
g) miRNAs with SEQ ID NO 1, 2, 5 and 12 (Set-No. SA-6) encompass stem-loop reverse primers having a nucleotide sequence according to SEQ ID NO: 25, 26, 29, 33 and forward primers having a nucleotide sequence according to SEQ ID NO: 36, 37, 40, 44 and the universal reverse primer having a nucleotide sequence according to SEQ ID NO: 47,
h) miRNAs with SEQ ID NO 1, 2, 5, 9 and 12 (Set-No. SA-13) encompass stem-loop reverse primers having a nucleotide sequence according to SEQ ID NO: 25, 26, 29, 31, 33 and forward primers having a nucleotide sequence according to SEQ ID NO: 36, 37, 40, 42, 44 and the universal reverse primer having a nucleotide sequence according to SEQ ID NO: 47, and/or
i) miRNAs with SEQ ID NO 1, 2, 9, 10 and 12 (Set-No. SA-15) encompass stem-loop reverse primers having a nucleotide sequence according to SEQ ID NO: 25, 26, 31, 32, 33 and forward primers having a nucleotide sequence according to SEQ ID NO: 36, 37, 42, 43, 44 and the universal reverse primer having a nucleotide sequence according to SEQ ID NO: 47.

Said means allow to determine the level (of each) of the at least three miRNAs in a blood sample isolated from a patient and, thus, to determine AD in the patient.

In particular,

it can be determined whether the patient (suspected of having AD) suffers from AD or not,
it can be determined whether AD is present in the patient (suspected of having AD),
it can be determined whether the patient is at risk for developing AD,
it can be determined whether AD is absent in the patient (suspected of having AD), and/or the course of AD in the patient (having AD) can be determined.

The at least three polynucleotides (polynucleotide probes) may be part of a microarray/biochip or may be attached to beads of a beads-based multiplex system.

The at least three polynucleotides (primers, in particular primer pairs) may be part of a RT-PCR system, a PCR-system, or a next generation sequencing system.

Said means may further comprise a microarray, a RT-PCT system, a PCR-system, a flow cytometer, a Luminex system and/or a next generation sequencing system.

The kit may also comprise

(iii) a container, and/or
(iv) a data carrier.

The data carrier may be a non-electronical data carrier, e.g. a graphical data carrier such as an information leaflet, an information sheet, a bar code or an access code, or an electronical data carrier such as a floppy disk, a compact disk (CD), a digital versatile disk (DVD), a microchip or another semiconductor-based electronical data carrier. The access code may allow the access to a database, e.g. an internet database, a centralized, or a decentralized database. The access code may also allow access to an application software that causes a computer to perform tasks for computer users or a mobile app which is a software designed to run on smartphones and other mobile devices.

Said data carrier may further comprise the at least three references, e.g. the reference level (of each) of the at least three miRNAs which level is determined herein. In case that the data carrier comprises an access code which allows the access to a database, said at least three references may be deposited in this database.

The data carrier may also comprise information or instructions on how to carry out the method according to the first aspect of the present invention.

Said kit may also comprise materials desirable from a commercial and user standpoint including a buffer(s), a reagent(s) and/or a diluent(s) for determining the level mentioned above.

In a sixth aspect, the present invention relates to a kit for determining Alzheimer's Disease in a patient which comprises:
(i) means for determining the level (of each) of at least three miRNAs, e.g. at least 3, 4, 5, or 6 miRNAs, or 7 miRNAs, in a blood sample isolated from a patient, wherein the at least three miRNAs are comprised in a set selected from the group consisting of:
   (a) SEQ ID NO: 1, SEQ ID NO: 2, and SEQ ID NO: 9,
   (b) SEQ ID NO: 1, SEQ ID NO: 2, and SEQ ID NO: 4,
   (c) SEQ ID NO: 1, SEQ ID NO: 2, and SEQ ID NO: 5,
   (d) SEQ ID NO: 1, SEQ ID NO: 2, and SEQ ID NO: 6,
   (e) SEQ ID NO: 1, SEQ ID NO: 2, SEQ ID NO: 6, and SEQ ID NO: 9,
   (f) SEQ ID NO: 1, SEQ ID NO: 2, SEQ ID NO: 5, and SEQ ID NO: 12,
   (g) SEQ ID NO: 1, SEQ ID NO: 2, SEQ ID NO: 3, SEQ ID NO: 6, SEQ ID NO: 8, and SEQ ID NO: 10,
   (h) SEQ ID NO: 1, SEQ ID NO: 2, SEQ ID NO: 3, SEQ ID NO: 6, and SEQ ID NO: 8,
   (i) SEQ ID NO: 1, SEQ ID NO: 2, SEQ ID NO: 5, SEQ ID NO: 10, SEQ ID NO: 12, and SEQ ID NO: 13,
   (j) SEQ ID NO: 1, SEQ ID NO: 2, SEQ ID NO: 3, SEQ ID NO: 5, SEQ ID NO: 6, and SEQ ID NO: 11,
   (k) SEQ ID NO: 1, SEQ ID NO: 2, SEQ ID NO: 3, SEQ ID NO: 4, SEQ ID NO: 7, SEQ ID NO: 8, and SEQ ID NO: 11,
   (l) SEQ ID NO: 1, SEQ ID NO: 2, SEQ ID NO: 12, and SEQ ID NO: 13,
   (m) SEQ ID NO: 1, SEQ ID NO: 2, SEQ ID NO: 5, SEQ ID NO: 9, and SEQ ID NO: 12,
   (n) SEQ ID NO: 1, SEQ ID NO: 2, SEQ ID NO: 7, SEQ ID NO: 9, and SEQ ID NO: 12,
   (o) SEQ ID NO: 1, SEQ ID NO: 2, SEQ ID NO: 9, SEQ ID NO: 10, and SEQ ID NO: 12,
   (p) SEQ ID NO: 1, SEQ ID NO: 2, and SEQ ID NO: 12,
   (q) SEQ ID NO: 1, SEQ ID NO: 2, and SEQ ID NO: 14,
   (r) SEQ ID NO: 1, SEQ ID NO: 2, SEQ ID NO: 9, and SEQ ID NO: 12,
   (s) SEQ ID NO: 1, SEQ ID NO: 2, and SEQ ID NO: 15,
   (t) SEQ ID NO: 1, SEQ ID NO: 3, SEQ ID NO: 4, SEQ ID NO: 7, and SEQ ID NO: 11,
   (u) SEQ ID NO: 1, SEQ ID NO: 3, SEQ ID NO: 5, SEQ ID NO: 8, SEQ ID NO: 9, and SEQ ID NO: 10,
   (v) SEQ ID NO: 1, SEQ ID NO: 3, SEQ ID NO: 4, SEQ ID NO: 5, SEQ ID NO: 7, SEQ ID NO: 11, and SEQ ID NO: 16,
   (w) SEQ ID NO: 1, SEQ ID NO: 3, SEQ ID NO: 5, SEQ ID NO: 6, SEQ ID NO: 8, SEQ ID NO: 16, and SEQ ID NO: 17,
   (x) SEQ ID NO: 1, SEQ ID NO: 3, SEQ ID NO: 5, SEQ ID NO: 8, SEQ ID NO: 16, SEQ ID NO: 17, and SEQ ID NO: 18, and
   (y) SEQ ID NO: 1, SEQ ID NO: 3, SEQ ID NO: 6, SEQ ID NO: 7, SEQ ID NO: 8, SEQ ID NO: 16, and SEQ ID NO: 17,
   and
(ii) optionally at least three references.

It should be noted that the at least three miRNAs have nucleotide sequences as shown in the sets according to (a) to (y). For example the set according to (a) comprises the miRNA having a nucleotide sequence according to SEQ ID NO: 1, the miRNA having a nucleotide sequence according to SEQ ID NO: 2, and the miRNA having a nucleotide sequence according to SEQ ID NO: 9.

The patient who's miRNA level can be determined may be a patient suspected of suffering from AD or a patient suffering from AD.

Especially, the kit is useful for conducting the method according to the second aspect of the present invention.

It is particularly preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for developing Alzheimer's Disease, and/or determining the course of Alzheimer's Disease in the patient. Thus, in view of the above, the kit according to the sixth aspect of the present invention is preferably designated as a kit

for diagnosing/determining whether a patient (suspected to be affected by AD) suffers from AD or not,
for determining the presence of AD in a patient (suspected to be affected by AD),
for determining the absence of AD in a patient (suspected to be affected by AD),
for determining the risk for developing AD in the patient, and/or
for determining the course of AD in a patient (having/suffering from AD).

The references may be any references which allow to determine AD in the patient. In particular, the references may be any references which allows to diagnose whether a patient (suspected of having AD) suffers from AD or not, to determine whether AD is present in the patient (suspected of having AD), to determine whether AD is absent in the patient (suspected of having AD), to determine whether the patient has a predisposition to develop AD or will likely develop AD (in the (near) future), and/or to determine the course of AD in the patient (having AD). In this respect, it is also referred to the preferred embodiments mentioned in the context of the second aspect of the present invention.

In particular, the means for determining the level (of each) of the at least three miRNAs comprise:

at least three polynucleotides (polynucleotide probes) (for each miRNA to be detected a specific polynucleotide probe), in particular according to the fourth aspect of the present invention,
at least three primer pairs (for each miRNA to be detected a specific primer pair), and/or
at least three polynucleotides (polynucleotide probes), in particular according to the fourth aspect of the present invention, and at least three antibodies capable of binding a hybrid of said polynucleotides (polynucleotide probes) and said miRNAs (for each miRNA to be detected a specific polynucleotide probe and an antibody capable of binding a hybrid of said polynucleotide probe and said miRNA).

As to the specific primers, in particular primer pairs, e.g. the primers having a nucleotide sequence according to SEQ ID NO: 25 to SEQ ID NO: 47, for detecting the level of the miRNAs comprised in a set in a blood sample isolated from a patient for determining Alzheimer's Disease in the patient, it is referred to the fifth aspect of the present invention. As to the dual-labelled probes having a nucleotide sequence according to SEQ ID NO: 48 to SEQ ID NO: 58, it is also referred to the fifth aspect of the present invention.

Said means allow to determine the level (of each) of the at least three miRNAs in a blood sample isolated from a patient and, thus, to determine AD in the patient.

In particular,

it can be determined whether the patient (suspected of having AD) suffers from AD or not,
it can be determined whether AD is present in the patient (suspected of having AD),
it can be determined whether AD is absent in the patient (suspected of having AD),
it can be determined whether the patient is at risk for developing AD, and/or the course of AD in the patient (having AD) can be determined.

The at least three polynucleotides (polynucleotide probes) may be part of a microarray/biochip or may be attached to beads of a beads-based multiplex system.

The at least three polynucleotides (primers, in particular primer pairs) may be part of a RT-PCR system, a PCR-system, or a next generation sequencing system.

Said means may further comprise a microarray, a RT-PCT system, a PCR-system, a flow cytometer, a Luminex system and/or a next generation sequencing system.

The kit may also comprise

(iii) a container, and/or
(iv) a data carrier.

The data carrier may be a non-electronical data carrier, e.g. a graphical data carrier such as an information leaflet, an information sheet, a bar code or an access code, or an electronical data carrier such as a floppy disk, a compact disk (CD), a digital versatile disk (DVD), a microchip or another semiconductor-based electronical data carrier. The access code may allow the access to a database, e.g. an internet database, a centralized, or a decentralized database. The access code may also allow access to an application software that causes a computer to perform tasks for computer users or a mobile app which is a software designed to run on smartphones and other mobile devices.

Said data carrier may further comprise the at least three references, e.g. the reference level (of each) of the at least three miRNAs which level is determined herein. In case that the data carrier comprises an access code which allows the access to a database, said at least three references may be deposited in this database.

The data carrier may also comprise information or instructions on how to carry out the method according to the second aspect of the present invention.

Said kit may also comprise materials desirable from a commercial and user standpoint including a buffer(s), a reagent(s) and/or a diluent(s) for determining the level mentioned above.

In a seventh aspect, the present invention relates to a(n) (in vitro) method for determining Alzheimer's Disease in a patient which comprises the steps of:
(i) determining the level (of each) of at least 3 miRNAs, e.g. at least 3, 4 or 5 miRNAs, or 6 miRNAs, comprised in a set in a blood sample isolated from a patient, wherein the first miRNA has a nucleotide sequence according to SEQ ID NO: 8 (hsa-miR-3909) or a nucleotide sequence having at least 90%, preferably at least 95%, more preferably at least 99%, i.e. at least 90, 91, 92, 93, 94, 95, 96, 97, 98, or 99%, sequence identity thereto, and
(ii) comparing the level (of each) of the at least 3 miRNAs comprised in the set with a reference level of said at least three miRNAs, wherein the comparison allows to determine Alzheimer's Disease in the patient.

It is preferred that the second and third miRNAs have nucleotide sequences selected from the group consisting of SEQ ID NO: 3, SEQ ID NO: 4, SEQ ID NO: 6, SEQ ID NO: 10, SEQ ID NO: 11, SEQ ID NO: 18, SEQ ID NO: 19, and SEQ ID NO: 20. Alternatively, the nucleotide sequences of the miRNAs according to SEQ ID NO: 3, SEQ ID NO: 4, SEQ ID NO: 6, SEQ ID NO: 10, SEQ ID NO: 11, SEQ ID NO: 18, SEQ ID NO: 19, or SEQ ID NO: 20 are nucleotide sequences having at least 90%, preferably at least 95%, more preferably at least 99%, i.e. at least 90, 91, 92, 93, 94, 95, 96, 97, 98, or 99%, sequence identity thereto.

It is more preferred the at least three miRNAs are comprised in a set selected from the group consisting of:
(a) SEQ ID NO: 3, SEQ ID NO: 4, SEQ ID NO: 8, SEQ ID NO: 10, SEQ ID NO: 11, and SEQ ID NO: 18,
(b) SEQ ID NO: 4, SEQ ID NO: 8, SEQ ID NO: 19, and SEQ ID NO: 20,
(c) SEQ ID NO: 8, SEQ ID NO: 18, and SEQ ID NO: 19, and
(d) SEQ ID NO: 3, SEQ ID NO: 6, and SEQ ID NO: 8.

It should be noted that the at least three miRNAs have nucleotide sequences as shown in the sets according to (a) to (d). For example the set according to (d) comprises the miRNA having a nucleotide sequence according to SEQ ID NO: 3, the miRNA having a nucleotide sequence according to SEQ ID NO: 6, and the miRNA having a nucleotide sequence according to SEQ ID NO: 8.

The patient who's miRNA level is determined may be a patient suspected of suffering from/having AD or a patient suffering from/having AD. In the latter case, the patient may be retested for AD (e.g. after a period of time).

The reference level may be any level which allows to determine AD in the patient or not. It may be obtained from a (control) subject (i.e. a subject different from the patient to be tested/diagnosed) or from the same patient. In the latter case, the patient may be retested for AD, e.g. in the form of a longitudinal monitoring. It may be determined that the patient is now affected by AD or still not affected by AD.

Preferably, the reference level is the level determined by measuring at least one reference blood sample from

at least one healthy subject, or
at least one subject having Alzheimer's Disease.

It is practicable to take one reference blood sample per subject for analysis. If additional reference blood samples are required, e.g. to determine the reference level in different reference blood samples, the same subject may be (re)tested. Said reference level may be an average reference level. It may be determined by measuring reference levels and calculating the “average” value (e.g. mean, median or modal value) thereof. It is preferred that the reference blood sample is from the same source (e.g. blood cell sample) than the blood sample isolated from the patient. It is further preferred that the reference level is obtained from a subject of the same gender (e.g. female or male) and/or of a similar age/phase of life (e.g. adults or elderly) than the patient to be tested or diagnosed.

In one embodiment, the determination of Alzheimer's Disease (AD) comprises diagnosing whether the patient (suspected of having AD) suffers from AD or not. In this case, the reference level may be any level which allows to determine whether the patient (suspected of having AD) suffers from AD or not. It may be obtained from a (control) subject (i.e. a subject different from the patient to be tested/diagnosed) or from the same patient. In the latter case, the patient may be retested for AD. In particular, the reference level is the level determined by measuring at least one reference blood sample from at least one healthy subject (see above).

Preferably,

the level of the miRNA having a nucleotide sequence selected from the group consisting of SEQ ID NO: 3 and SEQ ID NO: 18 to SEQ ID NO: 20 is above the reference level (determined by measuring at least one reference blood sample from at least one healthy subject) which indicates that the patient suffers from Alzheimer's Disease, and/or
the level of the miRNA having a nucleotide sequence selected from the group consisting of SEQ ID NO: 4, SEQ ID NO: 6, SEQ ID NO: 8, SEQ ID NO: 10, and SEQ ID NO: 11 is below the reference level (determined by measuring at least one reference blood sample from at least one healthy subject) which indicates that the patient suffers from Alzheimer's Disease.

In particular, the level of the miRNA is at least 0.4-fold, at least 0.5-fold, at least 0.6-fold or at least 0.7-fold, preferably at least 0.8-fold or at least 0.9-fold, more preferably at least 1.2-fold or at least 1.5-fold, and even more preferably at least 2.0-fold or at least 3.0-fold below/above the reference level. For example, the level of the miRNA is at least 0.4-fold, at least 0.5-fold, at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

In one another embodiment, the determination of Alzheimer's Disease (AD) comprises determining whether AD is present in the patient (suspected of having AD). In this case, the reference level may be any level which allows to determine whether AD is present in the patient (suspected of having AD). It may be obtained from a (control) subject (i.e. a subject different from the patient to be tested) or from the same patient. In the latter case, the patient may be retested for AD. In particular, the reference level is the level determined by measuring at least one reference blood sample from at least one healthy subject (see above).

Preferably,

the level of the miRNA having a nucleotide sequence selected from the group consisting of SEQ ID NO: 3 and SEQ ID NO: 18 to SEQ ID NO: 20 is above the reference level (determined by measuring at least one reference blood sample from at least one healthy subject) which indicates that Alzheimer's Disease is present in the patient, and/or
the level of the miRNA having a nucleotide sequence selected from the group consisting of SEQ ID NO: 4, SEQ ID NO: 6, SEQ ID NO: 8, SEQ ID NO: 10, and SEQ ID NO: 11 is below the reference level (determined by measuring at least one reference blood sample from at least one healthy subject) which indicates that Alzheimer's Disease is present in the patient.

In particular, the level of the miRNA is at least 0.4-fold, at least 0.5-fold, at least 0.6-fold or at least 0.7-fold, preferably at least 0.8-fold or at least 0.9-fold, more preferably at least 1.2-fold or at least 1.5-fold, and even more preferably at least 2.0-fold or at least 3.0-fold below/above the reference level. For example, the level of the miRNA is at least 0.4-fold, at least 0.5-fold, at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

In one another embodiment, the determination of Alzheimer's Disease (AD) comprises determining whether AD is absent in the patient (suspected of having AD). In this case, the reference level may be any level which allows to determine whether AD is absent in the patient (suspected of having AD). It may be obtained from a (control) subject (i.e. a subject different from the patient to be tested) or from the same patient. In the latter case, the patient may be retested for AD. In particular, the reference level is the level determined by measuring at least one reference blood sample from at least one healthy subject (see above).

Preferably,

the level of the miRNA having a nucleotide sequence selected from the group consisting of SEQ ID NO: 3 and SEQ ID NO: 18 to SEQ ID NO: 20 is comparable with the reference level (determined by measuring at least one reference blood sample from at least one healthy subject) which indicates that Alzheimer's Disease is absent in the patient,
the level of the miRNA having a nucleotide sequence selected from the group consisting of SEQ ID NO: 4, SEQ ID NO: 6, SEQ ID NO: 8, SEQ ID NO: 10, and SEQ ID NO: 11 is comparable with the reference level (determined by measuring at least one reference blood sample from at least one healthy subject) which indicates that Alzheimer's Disease is absent in the patient,
the level of the miRNA having a nucleotide sequence selected from the group consisting of SEQ ID NO: 3 and SEQ ID NO: 18 to SEQ ID NO: 20 is below the reference level (determined by measuring at least one reference blood sample from at least one subject having Alzheimer's Disease) which indicates that Alzheimer's Disease is absent in the patient, and/or
the level of the miRNA having a nucleotide sequence selected from the group consisting of SEQ ID NO: 4, SEQ ID NO: 6, SEQ ID NO: 8, SEQ ID NO: 10, and SEQ ID NO: 11 is above the reference level (determined by measuring at least one reference blood sample from at least one subject having Alzheimer's Disease) which indicates that Alzheimer's Disease is absent in the patient.

A level which is “comparable with” the reference level in this respect means that the level is no more than 15%, preferably no more than 10%, more preferably no more than 5%, above the reference level or the level is no more than 15%, preferably no more than 10%, more preferably no more than 5%, below the reference level.

Alternatively, a level which is comparable with the reference level in this respect means that the detected level variation is within the accuracy of a measurement. The accuracy of a measurement depends on the measurement method used.

In particular, the level of the miRNA is at least 0.4-fold, at least 0.5-fold, at least 0.6-fold or at least 0.7-fold, preferably at least 0.8-fold or at least 0.9-fold, more preferably at least 1.2-fold or at least 1.5-fold, and even more preferably at least 2.0-fold or at least 3.0-fold below/above the reference level. For example, the level of the miRNA is at least 0.4-fold, at least 0.5-fold, at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

In one another embodiment, the determination of Alzheimer's Disease (AD) comprises determining whether the patient is at risk for developing AD. In this case, the reference level may be any level which allows to determine whether the patient is at risk for developing AD or not. It may be obtained from a (control) subject (i.e. a subject different from the patient to be tested) or from the same patient. In the latter case, the patient may be retested for AD. In particular, the reference level is the level determined by measuring at least one reference blood sample from at least one healthy subject (see above). This determination may have the form of a screening method. Said screening method allows an early detection of AD and, thus, the identification of patients having a predisposition or a likelihood (e.g. on the basis of their physical and/or physiological conditions) to develop AD, e.g. in the near future, such as within the next 1, 2, 3, 4, or 5 years.

Preferably,

the level of the miRNA having a nucleotide sequence selected from the group consisting of SEQ ID NO: 3 and SEQ ID NO: 18 to SEQ ID NO: 20 is above the reference level (determined by measuring at least one reference blood sample from at least one healthy subject) which indicates that the patient is at risk for developing Alzheimer's Disease, and/or
the level of the miRNA having a nucleotide sequence selected from the group consisting of SEQ ID NO: 4, SEQ ID NO: 6, SEQ ID NO: 8, SEQ ID NO: 10, and SEQ ID NO: 11 is below the reference level (determined by measuring at least one reference blood sample from at least one healthy subject) which indicates that the patient is at risk for developing Alzheimer's Disease.

In particular, the level of the miRNA is at least 0.4-fold, at least 0.5-fold, at least 0.6-fold or at least 0.7-fold, preferably at least 0.8-fold or at least 0.9-fold, more preferably at least 1.2-fold or at least 1.5-fold, and even more preferably at least 2.0-fold or at least 3.0-fold below/above the reference level. For example, the level of the miRNA is at least 0.4-fold, at least 0.5-fold, at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

In another embodiment, the determination of Alzheimer's Disease (AD) comprises determining the course of AD in the patient (having AD). In this case, the reference level may be any level which allows to determine the course of AD in the patient (having AD). It may be obtained from a (control) subject (i.e. a subject different from the patient to be tested/diagnosed) or from the same patient. In the latter case, the patient may be retested for AD. It may be determined whether AD worsens in the patient, whether AD does not worsen/is stable in the patient, or whether AD improves in the patient. In particular, the reference level is the level determined by measuring at least one reference blood sample from at least one subject having AD (see above).

Especially, said determining comprises determining the level (of each) of the at least three miRNAs comprised in the set in the blood sample at a first point in time and in at least one further blood sample at a later point in time and comparing said levels determined at the different time points.

Preferably,

the level of the miRNA having a nucleotide sequence selected from the group consisting of SEQ ID NO: 3 and SEQ ID NO: 18 to SEQ ID NO: 20 which
(i) increases over time indicates that Alzheimer's Disease worsens in the patient,
(ii) does not change over time indicates that Alzheimer's Disease does not worsen/is stable in the patient, or
(iii) decreases over time indicates that Alzheimer's Disease improves in the patient, and/or
   the level of the miRNA having a nucleotide sequence selected from the group consisting of SEQ ID NO: 4, SEQ ID NO: 6, SEQ ID NO: 8, SEQ ID NO: 10, and SEQ ID NO: 11 which
(i) decreases over time indicates that Alzheimer's Disease worsens in the patient,
(ii) does not change over time indicates that Alzheimer's Disease does not worsen/is stable in the patient, or
(iii) increases over time indicates that Alzheimer's Disease improves in the patient.

As mentioned above, the detection of a decrease/an increase (dependent on the miRNA detected) of the level over time indicates that AD worsens in the patient. Preferably, said decrease/increase is at least 0.4-fold, at least 0.5-fold, at least 0.6-fold or at least 0.7-fold over time. More preferably, said decrease/increase is at least 0.8-fold or at least 0.9-fold over time. Even more preferably, said decrease/increase is at least 1.2-fold or at least 1.5-fold over time. Most preferably, said decrease/increase is at least 2.0-fold or at least 3.0-fold over time. For example, said decrease/increase may be determined over 1 year (12 months) or over 2 years (24 months).

As mentioned above, a level which does not change over time indicates that AD does not worsen/is stable in the patient. “Does not change over time” in this respect may mean that the level varies over time between 0 and <20%, e.g. 0, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 19.9, 19.99, or 19.999%. “Does not change over time” in this respect may also mean that the detected level variation is within the accuracy of a measurement. The accuracy of a measurement depends on the measurement method used. Preferably, the level is constant over time.

As mentioned above, the detection of an increase/a decrease (dependent on the miRNA detected) of the level over time indicates that AD improves in the patient. Preferably, said increase/decrease is at least 0.4-fold, at least 0.5-fold, at least 0.6-fold or at least 0.7-fold over time. More preferably, said increase/decrease is at least 0.8-fold or at least 0.9-fold over time. Even more preferably, said increase/decrease is at least 1.2-fold or at least 1.5-fold over time. Most preferably, said increase/decrease is at least 2.0-fold or at least 3.0-fold over time. For example, said increase/decrease may be determined over 1 year (12 months) or over 2 years (24 months).

The time period between the first point in time and the later point(s) in time preferably amounts to at least 1 day, at least 2 days, at least 3 days, at least 4 days, at least 5 days, at least 6 days, at least 7 days (1 week), at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months (1 year), at least 24 months (2 years), at least 3 years, at least 4 years, at least 5 years, at least 6 years, at least 7 years, at least 8 years, at least 9 years, or at least 10 years. For example, the patient may be routinely checked, e.g. once or twice a year. The patient may be (re)tested at 2, 3, 4, 5, 6 7, 8, 9, or 10 time points (first point in time and further point(s) in time). In addition to the determination of the course of AD, the treatment of this disease can be monitored. It is namely preferred that the patient receives or has received a treatment, in particular therapeutic treatment, of AD during the determination of the course of AD. The treatment of AD may be selected from the group consisting of the administration of a drug, cognitive training, ergotherapy, and psychotherapy. The drug may be selected from the group consisting of antidementives, antidepressants, and neuroleptics.

The patient may receive a treatment during the complete determination/monitoring process (e.g. the administration of a drug) or may receive a treatment before, at, or after a first point in time (e.g. the administration of a drug) and may be retested at a later point in time. In particular, said first point in time may be before the initiation of a treatment and said later point in time may be during the treatment and/or after the treatment. If the treatment encompasses the administration of a drug and the patient responds to said treatment, the drug administration may be continued, the dose of the drug may be reduced, or the drug administration may be stopped. If the treatment encompasses the administration of a drug and the patient does not respond to said treatment, the dose of the drug may be increased, the drug may be changed, or the therapy mode may be changed, e.g. from drug administration to cognitive training, ergotherapy, and/or psychotherapy.

As mentioned above, it is preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for developing Alzheimer's Disease, and/or determining the course of Alzheimer's Disease in the patient. Thus, in view of the above, the method according to the second aspect of the present invention is preferably designated as a method

for diagnosing/determining whether a patient (suspected to be affected by AD) suffers from AD or not,
for determining the presence of AD in a patient (suspected to be affected by AD),
for determining the absence of AD in a patient (suspected to be affected by AD),
for determining the risk for developing AD in a patient, and/or
for determining the course of AD in a patient (having/suffering from AD).

In an eight aspect, the present invention relates to a(n) (in vitro) method for determining Alzheimer's Disease in a patient comprising the steps of:
(i) determining the level (of each) of at least 3 miRNAs, e.g. at least 3, 4 or 5 miRNAs, or 6 miRNAs, in a blood sample isolated from a patient, wherein the at least three miRNAs are comprised in a set selected from the group consisting of:
   (a) SEQ ID NO: 3, SEQ ID NO: 4, SEQ ID NO: 8, SEQ ID NO: 10, SEQ ID NO: 11, and SEQ ID NO: 18,
   (b) SEQ ID NO: 4, SEQ ID NO: 8, SEQ ID NO: 19, and SEQ ID NO: 20,
   (c) SEQ ID NO: 8, SEQ ID NO: 18, and SEQ ID NO: 19, and
   (d) SEQ ID NO: 3, SEQ ID NO: 6, and SEQ ID NO: 8, and
(ii) comparing the level (of each) of the at least 3 miRNAs comprised in the set with a reference level of said at least three miRNAs, wherein the comparison allows to determine Alzheimer's Disease in the patient.

The patient who's miRNA level is determined may be a patient suspected of suffering from/having AD or a patient suffering from/having AD. In the latter case, the patient may be retested for AD (e.g. after a period of time).

The reference level may be any level which allows to determine AD in the patient or not. It may be obtained from a (control) subject (i.e. a subject different from the patient to be tested/diagnosed) or from the same patient. In the latter case, the patient may be retested for AD, e.g. in the form of a longitudinal monitoring. It may be determined that the patient is now affected by AD or still not affected by AD.

Preferably, the reference level is the level determined by measuring at least one reference blood sample from

at least one healthy subject, or
at least one subject having Alzheimer's Disease.

It is practicable to take one reference blood sample per subject for analysis. If additional reference blood samples are required, e.g. to determine the reference level in different reference blood samples, the same subject may be (re)tested. Said reference level may be an average reference level. It may be determined by measuring reference levels and calculating the “average” value (e.g. mean, median or modal value) thereof. It is preferred that the reference blood sample is from the same source (e.g. blood cell sample) than the blood sample isolated from the patient. It is further preferred that the reference level is obtained from a subject of the same gender (e.g. female or male) and/or of a similar age/phase of life (e.g. adults or elderly) than the patient to be tested or diagnosed.

It is preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for developing Alzheimer's Disease, and/or determining the course of Alzheimer's Disease in the patient. Thus, in view of the above, the method according to the eight aspect of the present invention is preferably designated as a method

for diagnosing/determining whether a patient (suspected to be affected by AD) suffers from AD or not,
for determining the presence of AD in a patient (suspected to be affected by AD),
for determining the absence of AD in a patient (suspected to be affected by AD),
for determining the risk for developing AD in a patient, and/or
for determining the course of AD in a patient (having (suffering from AD).

As to the preferred embodiments with respect to diagnosing whether the patient suffers from Alzheimer's Disease, determining whether Alzheimer's Disease is present in the patient, determining whether Alzheimer's Disease is absent in the patient, and/or determining the course of Alzheimer's Disease in the patient, it is referred to the seventh aspect of the present invention.

In a ninth aspect, the present invention relates to the (in vitro) use at least three polynucleotides (probes or primers, in particular primer pairs) for detecting at least three miRNAs, e.g. at least 3, 4 or 5 miRNAs, or 6 miRNAs, comprised in a set in a blood sample isolated from a patient for determining Alzheimer's Disease in the patient, wherein the first miRNA has a nucleotide sequence according to SEQ ID NO: 8 (hsa-miR-3909).

It is preferred that the second and third miRNAs have nucleotide sequences selected from the group consisting of SEQ ID NO: 3, SEQ ID NO: 4, SEQ ID NO: 6, SEQ ID NO: 10, SEQ ID NO: 11, SEQ ID NO: 18, SEQ ID NO: 19, and SEQ ID NO: 20. Alternatively, the nucleotide sequences of the miRNAs according to SEQ ID NO: 3, SEQ ID NO: 4, SEQ ID NO: 6, SEQ ID NO: 10, SEQ ID NO: 11, SEQ ID NO: 18, SEQ ID NO: 19, or SEQ ID NO: 20 are nucleotide sequences having at least 90%, preferably at least 95%, more preferably at least 99%, i.e. at least 90, 91, 92, 93, 94, 95, 96, 97, 98, or 99%, sequence identity thereto. It is more preferred the at least three miRNAs are comprised in a set selected from the group consisting of:
(a) SEQ ID NO: 3, SEQ ID NO: 4, SEQ ID NO: 8, SEQ ID NO: 10, SEQ ID NO: 11, and SEQ ID NO: 18,
(b) SEQ ID NO: 4, SEQ ID NO: 8, SEQ ID NO: 19, and SEQ ID NO: 20,
(c) SEQ ID NO: 8, SEQ ID NO: 18, and SEQ ID NO: 19, and
(d) SEQ ID NO: 3, SEQ ID NO: 6, and SEQ ID NO: 8.

The patient who's miRNAs are detected may be a patient suspected of suffering from AD or a patient suffering from AD.

The at least three polynucleotides may be probes or primers, in particular primer pairs.

Preferably,
(i) the polynucleotides are at least partially (reverse) complementary, preferably (reverse) complementary, to the (respective) miRNAs mentioned above, or
(ii) the polynucleotides have at least 90%, preferably at least 95%, more preferably at least 99%, i.e. at least 90, 91, 92, 93, 94, 95, 96, 97, 98, or 99%, sequence identity to the polynucleotides according to (i).

It is particularly preferred that the polynucleotides as defined in (ii) have at least 90%, preferably at least 95%, more preferably at least 99%, i.e. at least 90, 91, 92, 93, 94, 95, 96, 97, 98, or 99%, sequence identity over a continuous stretch of at least 10, 11, 12, 13, 14, 15, 16, 17, 18, 19, 20, 21, 22, 23, 24, 25, or more nucleotides, preferably over the whole length, to the polynucleotides according to (i).

As to the polynucleotide variants, it is referred to the third aspect of the present invention.

The polynucleotides (probes or primers, in particular primer pair) described above are useful for conducting the method according to the seventh aspect of the present invention.

It is particularly preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for developing Alzheimer's Disease, and/or determining the course of Alzheimer's Disease in the patient.

In a tenth aspect, the present invention the present invention relates to the (in vitro) use of at least three polynucleotides (probes or primers, in particular primer pairs) for detecting at least three miRNAs, e.g. at least 3, 4 or 5 miRNAs, or 6 miRNAs, in a blood sample isolated from a patient for determining Alzheimer's Disease in the patient, wherein the at least three miRNAs are comprised in a set selected from the group consisting of:
(a) SEQ ID NO: 3, SEQ ID NO: 4, SEQ ID NO: 8, SEQ ID NO: 10, SEQ ID NO: 11, and SEQ ID NO: 18,
(b) SEQ ID NO: 4, SEQ ID NO: 8, SEQ ID NO: 19, and SEQ ID NO: 20,
(c) SEQ ID NO: 8, SEQ ID NO: 18, and SEQ ID NO: 19, and
(d) SEQ ID NO: 3, SEQ ID NO: 6, and SEQ ID NO: 8.

The patient who's miRNAs are detected may be a patient suspected of suffering from AD or a patient suffering from AD.

The at least three polynucleotides may be probes or primers, in particular primer pairs.

Preferably,
(i) the polynucleotides are at least partially (reverse) complementary, preferably (reverse) complementary, to the (respective) miRNAs mentioned above, or
(ii) the polynucleotides have at least 90%, preferably at least 95%, more preferably at least 99%, i.e. at least 90, 91, 92, 93, 94, 95, 96, 97, 98, or 99%, sequence identity to the polynucleotides according to (i).

It is particularly preferred that the polynucleotides as defined in (ii) have at least 90%, preferably at least 95%, more preferably at least 99%, i.e. at least 90, 91, 92, 93, 94, 95, 96, 97, 98, or 99%, sequence identity over a continuous stretch of at least 10, 11, 12, 13, 14, 15, 16, 17, 18, 19, 20, 21, 22, 23, 24, 25, or more nucleotides, preferably over the whole length, to the polynucleotides according to (i).

As to the polynucleotide variants, it is referred to the third aspect of the present invention.

The polynucleotides (probes or primers, in particular primer pair) described above are useful for conducting the method according to the eight aspect of the present invention.

It is particularly preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for developing Alzheimer's Disease, and/or determining the course of Alzheimer's Disease in the patient.

In an eleventh aspect, the present invention relates to (the use of) a kit for determining Alzheimer's Disease in a patient comprising:
(i) means for determining the level (of each) of at least three miRNAs, e.g. at least 3, 4 or 5 miRNAs, or 6 miRNAs, comprised in a set in a blood sample isolated from a patient, wherein the first miRNA has a nucleotide sequence according to SEQ ID NO: 8 (hsa-miR-3909) or a nucleotide sequence having at least 90%, preferably at least 95%, more preferably at least 99%, i.e. at least 90, 91, 92, 93, 94, 95, 96, 97, 98, or 99%, sequence identity thereto, and
(ii) optionally at least three references.

It is preferred that the second and third miRNAs have nucleotide sequences selected from the group consisting of SEQ ID NO: 3, SEQ ID NO: 4, SEQ ID NO: 6, SEQ ID NO: 10, SEQ ID NO: 11, SEQ ID NO: 18, SEQ ID NO: 19, and SEQ ID NO: 20. Alternatively, the nucleotide sequences of the miRNAs according to SEQ ID NO: 3, SEQ ID NO: 4, SEQ ID NO: 6, SEQ ID NO: 10, SEQ ID NO: 11, SEQ ID NO: 18, SEQ ID NO: 19, or SEQ ID NO: 20 are nucleotide sequences having at least 90%, preferably at least 95%, more preferably at least 99%, i.e. at least 90, 91, 92, 93, 94, 95, 96, 97, 98, or 99%, sequence identity thereto.

It is more preferred the at least three miRNAs are comprised in a set selected from the group consisting of:
(a) SEQ ID NO: 3, SEQ ID NO: 4, SEQ ID NO: 8, SEQ ID NO: 10, SEQ ID NO: 11, and SEQ ID NO: 18,
(b) SEQ ID NO: 4, SEQ ID NO: 8, SEQ ID NO: 19, and SEQ ID NO: 20,
(c) SEQ ID NO: 8, SEQ ID NO: 18, and SEQ ID NO: 19, and
(d) SEQ ID NO: 3, SEQ ID NO: 6, and SEQ ID NO: 8.

The patient who's miRNA level is determined may be a patient suspected of suffering from AD or a patient suffering from AD.

Especially, the kit is useful for conducting the method according to the seventh aspect of the present invention.

It is particularly preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for developing Alzheimer's Disease, and/or determining the course of Alzheimer's Disease in the patient.

The references may be any references which allow to determine AD in the patient. In particular, the references may be any references which allows to diagnose whether a patient (suspected of having AD) suffers from AD or not, to determine whether AD is present in the patient (suspected of having AD), to determine whether AD is absent in the patient (suspected of having AD), to determine whether the patient has a predisposition to develop AD or will likely develop AD (in the (near) future), and/or to determine the course of AD in the patient (having AD). In this respect, it is also referred to the preferred embodiments mentioned in the context of the seventh aspect of the present invention.

In particular, the means for determining the level (of each) of the at least three miRNAs comprise:

at least three polynucleotides (polynucleotide probes) (for each miRNA to be detected a specific polynucleotide probe), in particular according to the ninth aspect of the present invention,
at least three primer pairs (for each miRNA to be detected a specific primer pair), and/or
at least three polynucleotides (polynucleotide probes), in particular according to the ninth aspect of the present invention, and at least three antibodies capable of binding a hybrid of said polynucleotides (polynucleotide probes) and said miRNAs (for each miRNA to be detected a specific polynucleotide probe and an antibody capable of binding a hybrid of said polynucleotide probe and said miRNA).

Said means allow to determine the level (of each) of the at least three miRNAs in a blood sample isolated from a patient and, thus, to determine AD in the patient.

In particular,

it can be determined whether the patient (suspected of having AD) suffers from AD or not,
it can be determined whether AD is present in the patient (suspected of having AD),
it can be determined whether AD is absent in the patient (suspected of having AD),
it can be determined whether the patient is at risk for developing AD, and/or the course of AD in the patient (having AD) can be determined.

The at least three polynucleotides (polynucleotide probes) may be part of a microarray/biochip or may be attached to beads of a beads-based multiplex system.

The at least three polynucleotides (primers, in particular primer pairs) may be part of a RT-PCR system, a PCR-system, or a next generation sequencing system.

Said means may further comprise a microarray, a RT-PCT system, a PCR-system, a flow cytometer, a Luminex system and/or a next generation sequencing system.

The kit may also comprise
(iii) a container, and/or
(iv) a data carrier.

The data carrier may be a non-electronical data carrier, e.g. a graphical data carrier such as an information leaflet, an information sheet, a bar code or an access code, or an electronical data carrier such as a floppy disk, a compact disk (CD), a digital versatile disk (DVD), a microchip or another semiconductor-based electronical data carrier. The access code may allow the access to a database, e.g. an internet database, a centralized, or a decentralized database. The access code may also allow access to an application software that causes a computer to perform tasks for computer users or a mobile app which is a software designed to run on smartphones and other mobile devices.

Said data carrier may further comprise the at least three references, e.g. the reference level (of each) of the level of the at least three miRNAs determined herein. In case that the data carrier comprises an access code which allows the access to a database, said at least three references may be deposited in this database.

The data carrier may also comprise information or instructions on how to carry out the method according to the seventh aspect of the present invention.

Said kit may also comprise materials desirable from a commercial and user standpoint including a buffer(s), a reagent(s) and/or a diluent(s) for determining the level mentioned above.

In a twelfth aspect, the present invention relates to (the use of) a kit for determining Alzheimer's Disease in a patient comprising:
(i) means for determining the level (of each) of at least three miRNAs, e.g. at least 3, 4 or 5 miRNAs, or 6 miRNAs, in a blood sample isolated from a patient, wherein the at least three miRNAs are comprised in a set selected from the group consisting of:
   (a) SEQ ID NO: 3, SEQ ID NO: 4, SEQ ID NO: 8, SEQ ID NO: 10, SEQ ID NO: 11, and SEQ ID NO: 18,
   (b) SEQ ID NO: 4, SEQ ID NO: 8, SEQ ID NO: 19, and SEQ ID NO: 20,
   (c) SEQ ID NO: 8, SEQ ID NO: 18, and SEQ ID NO: 19, and
   (d) SEQ ID NO: 3, SEQ ID NO: 6, and SEQ ID NO: 8, and
(ii) optionally at least three references.

The patient who's miRNA level is determined may be a patient suspected of suffering from AD or a patient suffering from AD.

Especially, the kit is useful for conducting the method according to the eight aspect of the present invention.

It is particularly preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for developing Alzheimer's Disease, and/or determining the course of Alzheimer's Disease in the patient.

In particular, the means for determining the level (of each) of the at least three miRNAs comprise:

at least three polynucleotides (polynucleotide probes) (for each miRNA to be detected a specific polynucleotide probe), in particular according to the tenth aspect of the present invention,
at least three primer pairs (for each miRNA to be detected a specific primer pair), and/or
at least three polynucleotides (polynucleotide probes), in particular according to the tenth aspect of the present invention, and at least three antibodies capable of binding a hybrid of said polynucleotides (polynucleotide probes) and said miRNAs (for each miRNA to be detected a specific polynucleotide probe and an antibody capable of binding a hybrid of said polynucleotide probe and said miRNA).

Said means allow to determine the level (of each) of the at least three miRNAs in a blood sample isolated from a patient and, thus, to determine AD in the patient.

In particular,

it can be determined whether the patient (suspected of having AD) suffers from AD or not,
it can be determined whether AD is present in the patient (suspected of having AD),
it can be determined whether AD is absent in the patient (suspected of having AD),
it can be determined whether the patient is at risk for developing AD, and/or the course of AD in the patient (having AD) can be determined.

The at least three polynucleotides (polynucleotide probes) may be part of a microarray/biochip or may be attached to beads of a beads-based multiplex system.

The at least three polynucleotides (primers, in particular primer pairs) may be part of a RT-PCR system, a PCR-system, or a next generation sequencing system.

Said means may further comprise a microarray, a RT-PCT system, a PCR-system, a flow cytometer, a Luminex system and/or a next generation sequencing system.

The kit may also comprise

(iii) a container, and/or
(iv) a data carrier.

The data carrier may comprise information or instructions on how to carry out the method according to the eight aspect of the present invention.

In a thirteenth aspect, the present invention relates to a(n) (in vitro) method for determining Alzheimer's Disease in a patient comprising the steps of:
(i) determining the level (of each) of at least 3 miRNAs, e.g. at least 3, 4 or 5 miRNAs, or 6 miRNAs, in a blood sample isolated from a patient, wherein the at least three miRNAs are comprised in a set selected from the group consisting of:
   (a) SEQ ID NO: 2, SEQ ID NO: 3, and SEQ ID NO: 10, and
   (b) SEQ ID NO: 2, SEQ ID NO: 9, SEQ ID NO: 21, SEQ ID NO: 22, SEQ ID NO: 23, and SEQ ID NO: 24
   and
(ii) comparing the level (of each) of the at least 3 miRNAs comprised in the set with a reference level of said at least three miRNAs, wherein the comparison allows to determine Alzheimer's Disease in the patient.

It should be noted that the at least three miRNAs have nucleotide sequences as shown in the sets according to (a) and (b).

Alternatively, the nucleotide sequences of the miRNAs according to SEQ ID NO: 2, SEQ ID NO: 3, SEQ ID NO: 9, SEQ ID NO: 10, SEQ ID NO: 21, SEQ ID NO: 22, SEQ ID NO: 23, or SEQ ID NO: 24 are nucleotide sequences having at least 90%, preferably at least 95%, more preferably at least 99%, i.e. at least 90, 91, 92, 93, 94, 95, 96, 97, 98, or 99%, sequence identity thereto.

The patient who's miRNA level is determined may be a patient suspected of suffering from/having AD or a patient suffering from/having AD. In the latter case, the patient may be retested for AD (e.g. after a period of time).

Preferably, the reference level is the level determined by measuring at least one reference blood sample from

at least one healthy subject, or
at least one subject having Alzheimer's Disease.

In one embodiment, the determination of Alzheimer's Disease (AD) comprises diagnosing whether the patient (suspected of having AD) suffers from AD or not.

Preferably,

the level of the miRNA having a nucleotide sequence selected from the group consisting of SEQ ID NO: 3 and SEQ ID NO: 22 is above the reference level (determined by measuring at least one reference blood sample from at least one healthy subject) which indicates that the patient suffers from Alzheimer's Disease, and/or
the level of the miRNA having a nucleotide sequence selected from the group consisting of SEQ ID NO: 2, SEQ ID NO: 9, SEQ ID NO: 10, SEQ ID NO: 21, SEQ ID NO: 23, and SEQ ID NO: 24 is below the reference level (determined by measuring at least one reference blood sample from at least one healthy subject) which indicates that the patient suffers from Alzheimer's Disease.

In one another embodiment, the determination of Alzheimer's Disease (AD) comprises determining whether AD is present in the patient (suspected of having AD).

Preferably,

the level of the miRNA having a nucleotide sequence selected from the group consisting of SEQ ID NO: 3 and SEQ ID NO: 22 is above the reference level (determined by measuring at least one reference blood sample from at least one healthy subject) which indicates that Alzheimer's Disease is present in the patient, and/or
the level of the miRNA having a nucleotide sequence selected from the group consisting of SEQ ID NO: 2, SEQ ID NO: 9, SEQ ID NO: 10, SEQ ID NO: 21, SEQ ID NO: 23, and SEQ ID NO: 24 is below the reference level (determined by measuring at least one reference blood sample from at least one healthy subject) which indicates that Alzheimer's Disease is present in the patient.

In one another embodiment, the determination of Alzheimer's Disease (AD) comprises determining whether AD is absent in the patient (suspected of having AD).

Preferably,

the level of the miRNA having a nucleotide sequence selected from the group consisting of SEQ ID NO: 3 and SEQ ID NO: 22 is comparable with the reference level (determined by measuring at least one reference blood sample from at least one healthy subject) which indicates that Alzheimer's Disease is absent in the patient,
the level of the miRNA having a nucleotide sequence selected from the group consisting of SEQ ID NO: 2, SEQ ID NO: 9, SEQ ID NO: 10, SEQ ID NO: 21, SEQ ID NO: 23, and SEQ ID NO: 24 is comparable with the reference level (determined by measuring at least one reference blood sample from at least one healthy subject) which indicates that Alzheimer's Disease is absent in the patient,
the level of the miRNA having a nucleotide sequence selected from the group consisting of SEQ ID NO: 3 and SEQ ID NO: 22 is below the reference level (determined by measuring at least one reference blood sample from at least one subject having Alzheimer's Disease) which indicates that Alzheimer's Disease is absent in the patient, and/or
the level of the miRNA having a nucleotide sequence selected from the group consisting of SEQ ID NO: 2, SEQ ID NO: 9, SEQ ID NO: 10, SEQ ID NO: 21, SEQ ID NO: 23, and SEQ ID NO: 24 is above the reference level (determined by measuring at least one reference blood sample from at least one subject having Alzheimer's Disease) which indicates that Alzheimer's Disease is absent in the patient.

In one another embodiment, the determination of Alzheimer's Disease (AD) comprises determining whether the patient is at risk for developing AD.

Preferably,

the level of the miRNA having a nucleotide sequence selected from the group consisting of SEQ ID NO: 3 and SEQ ID NO: 22 is above the reference level (determined by measuring at least one reference blood sample from at least one healthy subject) which indicates that the patient is at risk for developing Alzheimer's Disease, and/or
the level of the miRNA having a nucleotide sequence selected from the group consisting of SEQ ID NO: 2, SEQ ID NO: 9, SEQ ID NO: 10, SEQ ID NO: 21, SEQ ID NO: 23, and SEQ ID NO: 24 is below the reference level (determined by measuring at least one reference blood sample from at least one healthy subject) which indicates that the patient is at risk for developing Alzheimer's Disease.

The meaning of the term “comparable with” is described above, for example in the context of the first aspect of the present invention.

In particular, the level of the miRNA is at least 0.4-fold, at least 0.5-fold, at least 0.6-fold or at least 0.7-fold, preferably at least 0.8-fold or at least 0.9-fold, more preferably at least 1.2-fold or at least 1.5-fold, and even more preferably at least 2.0-fold or at least 3.0-fold below/above the reference level. For example, the level of the miRNA is at least 0.4-fold, at least 0.5-fold, at least 0.6-fold, at least 0.7-fold, at least 0.8-fold, at least 0.9-fold, at least 1.0-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, or at least 3.0-fold below/above the reference level.

In another embodiment, the determination of Alzheimer's Disease (AD) comprises determining the course of AD in the patient (having AD).

Especially, said determining comprises determining the level (of each) of the at least three miRNAs comprised in the set in the blood sample at a first point in time and in at least one further blood sample at a later point in time and comparing said levels determined at the different time points.

Preferably,

the level of the miRNA having a nucleotide sequence selected from the group consisting of SEQ ID NO: 3 and SEQ ID NO: 22 which
(i) increases over time indicates that Alzheimer's Disease worsens in the patient,
(ii) does not change over time indicates that Alzheimer's Disease does not worsen/is stable in the patient, or
(iii) decreases over time indicates that Alzheimer's Disease improves in the patient,
   and/or
   the level of the miRNA having a nucleotide sequence selected from the group consisting of SEQ ID NO: 2, SEQ ID NO: 9, SEQ ID NO: 10, SEQ ID NO: 21, SEQ ID NO: 23, and SEQ ID NO: 24 which
(i) decreases over time indicates that Alzheimer's Disease worsens in the patient,
(ii) does not change over time indicates that Alzheimer's Disease does not worsen/is stable in the patient, or
(iii) increases over time indicates that Alzheimer's Disease improves in the patient.

As mentioned above, the detection of a decrease/an increase (dependent on the miRNA detected) of the level over time indicates that AD worsens in the patient. Preferably, said decrease/increase is at least 0.4-fold, at least 0.5-fold, at least 0.6-fold or at least 0.7-fold over time. More preferably, said decrease/increase is at least 0.8-fold or at least 0.9-fold over time. Even more preferably, said decrease/increase is at least 1.2-fold or at least 1.5-fold over time. Most preferably, said decrease/increase is at least 2.0-fold or at least 3.0-fold over time. For example, said decrease/increase may be determined over 1 year (12 months) or over 2 years (24 months).

The meaning of the term “does not change over time” is described above, for example in the context of the first aspect of the present invention.

As mentioned above, the detection of an increase/a decrease (dependent on the miRNA detected) of the level over time indicates that AD improves in the patient. Preferably, said increase/decrease is at least 0.4-fold, at least 0.5-fold, at least 0.6-fold or at least 0.7-fold over time. More preferably, said increase/decrease is at least 0.8-fold or at least 0.9-fold over time. Even more preferably, said increase/decrease is at least 1.2-fold or at least 1.5-fold over time. Most preferably, said increase/decrease is at least 2.0-fold or at least 3.0-fold over time. For example, said increase/decrease may be determined over 1 year (12 months) or over 2 years (24 months).

The time period between the first point in time and the later point(s) in time preferably amounts to at least 1 day, at least 2 days, at least 3 days, at least 4 days, at least 5 days, at least 6 days, at least 7 days (1 week), at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months (1 year), at least 24 months (2 years), at least 3 years, at least 4 years, at least 5 years, at least 6 years, at least 7 years, at least 8 years, at least 9 years, or at least 10 years. For example, the patient may be routinely checked, e.g. once or twice a year. The patient may be (re)tested at 2, 3, 4, 5, 6 7, 8, 9, or 10 time points (first point in time and further point(s) in time).

In addition to the determination of the course of AD, the treatment of this disease can be monitored. It is namely preferred that the patient receives or has received a treatment, in particular therapeutic treatment, of AD during the determination of the course of AD. The treatment of AD may be selected from the group consisting of the administration of a drug, cognitive training, ergotherapy, and psychotherapy. The drug may be selected from the group consisting of antidementives, antidepressants, and neuroleptics.

The patient may receive a treatment during the complete determination/monitoring process (e.g. the administration of a drug) or may receive a treatment before, at, or after a first point in time (e.g. the administration of a drug) and may be retested at a later point in time. In particular, said first point in time may be before the initiation of a treatment and said later point in time may be during the treatment and/or after the treatment. If the treatment encompasses the administration of a drug and the patient responds to said treatment, the drug administration may be continued, the dose of the drug may be reduced, or the drug administration may be stopped. If the treatment encompasses the administration of a drug and the patient does not respond to said treatment, the dose of the drug may be increased, the drug may be changed, or the therapy mode may be changed, e.g. from drug administration to cognitive training, ergotherapy, and/or psychotherapy.

In a fourteenth aspect, the present invention relates to the (in vitro) use of at least three polynucleotides (probes or primers, in particular primer pairs) for detecting at least three miRNAs, e.g. at least 3, 4 or 5 miRNAs, or 6 miRNAs, in a blood sample isolated from a patient for determining Alzheimer's Disease in the patient, wherein the at least three miRNAs are comprised in a set selected from the group consisting of:
(a) SEQ ID NO: 2, SEQ ID NO: 3, and SEQ ID NO: 10, and
(b) SEQ ID NO: 2, SEQ ID NO: 9, SEQ ID NO: 21, SEQ ID NO: 22, SEQ ID NO: 23, and SEQ ID NO: 24

The patient who's miRNAs are detected may be a patient suspected of suffering from AD or a patient suffering from AD.

The at least three polynucleotides may be probes or primers, in particular primer pairs.

Preferably,
(i) the polynucleotides are at least partially (reverse) complementary, preferably (reverse) complementary, to the (respective) miRNAs mentioned above, or
(ii) the polynucleotides have at least 90%, preferably at least 95%, more preferably at least 99%, i.e. at least 90, 91, 92, 93, 94, 95, 96, 97, 98, or 99%, sequence identity to the polynucleotides according to (i).

As to the polynucleotide variants, it is referred to the third aspect of the present invention.

The polynucleotides (probes or primers, in particular primer pair) described above are useful for conducting the method according to the thirteenth aspect of the present invention.

It is particularly preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for developing Alzheimer's Disease, and/or determining the course of Alzheimer's Disease in the patient.

In a fifteenth aspect, the present invention relates to (the use of) a kit for determining Alzheimer's Disease in a patient comprising:
(i) means for determining the level (of each) of at least three miRNAs, e.g. at least 3, 4 or 5 miRNAs, or 6 miRNAs, in a blood sample isolated from a patient, wherein the at least three miRNAs are comprised in a set selected from the group consisting of:
   (a) SEQ ID NO: 2, SEQ ID NO: 3, and SEQ ID NO: 10, and
   (b) SEQ ID NO: 2, SEQ ID NO: 9, SEQ ID NO: 21, SEQ ID NO: 22, SEQ ID NO: 23, and SEQ ID NO: 24
   and
(ii) optionally at least three references.

The patient who's miRNA level is determined may be a patient suspected of suffering from AD or a patient suffering from AD.

Especially, the kit is useful for conducting the method according to the thirteenth aspect of the present invention.

It is particularly preferred that the determination of Alzheimer's Disease comprises diagnosing whether the patient suffers from Alzheimer's Disease, determining whether Alzheimer's Disease is present in the patient, determining whether Alzheimer's Disease is absent in the patient, determining whether the patient is at risk for developing Alzheimer's Disease, and/or determining the course of Alzheimer's Disease in the patient.

In particular, the means for determining the level (of each) of the at least three miRNAs comprise:

at least three polynucleotides (polynucleotide probes) (for each miRNA to be detected a specific polynucleotide probe), in particular according to the fourteenth aspect of the present invention,
at least three primer pairs (for each miRNA to be detected a specific primer pair), and/or
at least three polynucleotides (polynucleotide probes), in particular according to the fourteenth aspect of the present invention, and at least three antibodies capable of binding a hybrid of said polynucleotides (polynucleotide probes) and said miRNAs (for each miRNA to be detected a specific polynucleotide probe and an antibody capable of binding a hybrid of said polynucleotide probe and said miRNA).

Said means allow to determine the level (of each) of the at least three miRNAs in a blood sample isolated from a patient and, thus, to determine AD in the patient.

In particular,

it can be determined whether the patient (suspected of having AD) suffers from AD or not,
it can be determined whether AD is present in the patient (suspected of having AD),
it can be determined whether AD is absent in the patient (suspected of having AD),
it can be determined whether the patient is at risk for developing AD, and/or
the course of AD in the patient (having AD) can be determined.

The at least three polynucleotides (polynucleotide probes) may be part of a microarray/biochip or may be attached to beads of a beads-based multiplex system.

The at least three polynucleotides (primers, in particular primer pairs) may be part of a RT-PCR system, a PCR-system, or a next generation sequencing system.

Said means may further comprise a microarray, a RT-PCT system, a PCR-system, a flow cytometer, a Luminex system and/or a next generation sequencing system.

The kit may also comprise
(iii) a container, and/or
(iv) a data carrier.

The data carrier may comprise information or instructions on how to carry out the method according to the thirteenth aspect of the present invention.

The determination of the level (of each) of at least three miRNAs according to the first, second, seventh, eighth, or thirteenth aspect of the present invention may be carried out by any convenient means for determining the level of a nucleotide sequence such as miRNA. For this purpose, qualitative, semi-quantitative and quantitative detection methods can be used. Quantitative detection methods are preferred. A variety of techniques are well known to the person skilled in the art. For example, the level (of each) of at least three miRNAs can be determined in the methods according to the first, second, seventh, eighth, or thirteenth aspect of the present invention by nucleic acid hybridization, nucleic acid amplification, polymerase extension, sequencing, mass spectroscopy, an immunochemical method, or any combination thereof.

Preferably,
(i) the nucleic acid hybridization is performed using a microarray/biochip, or using in situ hybridization,
(ii) the nucleic acid amplification is performed using real-time PCR (RT-PCR) or real-time quantitative PCR (RT-qPCR),
(iii) the sequencing is next generation sequencing, or
(iv) the immunochemical method is an enzyme linked immunosorbent assay (ELISA).

Nucleic acid amplification, for example, may be performed using real time polymerase chain reaction (RT-PCR) such as real time quantitative PCR (RT-qPCR). The real time polymerase chain reaction (RT-PCR) may include the following steps: (i) extracting total RNA from the blood sample isolated from the patient, (ii) obtaining cDNA samples by RNA reverse transcription (RT) reaction using miRNA-specific primers, (iii) designing miRNA-specific cDNA forward primers and providing universal reverse primers to amplify the cDNA via polymerase chain reaction (PCR), (iv) adding a fluorescent probe to conduct PCR, and (v) detecting and comparing the variation in levels of miRNAs in the blood sample isolated from the patient relative to those of miRNAs in a reference blood sample isolated from a (control) subject.

A variety of kits and protocols to determine the miRNA level by real time polymerase chain reaction (RT-PCR) such as real time quantitative PCR (RT-qPCR) are available. For example, reverse transcription of miRNAs may be performed using the TaqMan MicroRNA Reverse Transcription Kit (Applied Biosystems) according to manufacturer's recommendations.

Nucleic acid hybridization, for example, may be performed using a microarray/biochip or in situ hybridization. For nucleic acid hybridization, for example, the polynucleotides (probes) described herein with complementarity to the corresponding miRNAs to be detected are attached to a solid phase to generate a microarray/biochip. Said microarray/biochip is then incubated with miRNAs, isolated (e.g. extracted) from the blood sample, which may be labelled or unlabelled. Upon hybridization of the labelled miRNAs to the complementary polynucleotide sequences on the microarray/biochip, the success of hybridisation may be controlled and the intensity of hybridization may be determined via the hybridisation signal of the label in order to determine the level of each tested miRNA in said blood sample.

Alternatively, the miRNA level may be determined using an immunochemical method, e.g. using an ELISA. Said method may include the following steps: (i) isolating miRNAs from a blood sample, (ii) hybridizing polynucleotide probes (complementary) to the miRNAs to obtain hybrids of said polynucleotides probes and said miRNAs, and (iii) binding said hybrids to antibodies capable of specifically binding hybrids of said polynucleotide probes and said miRNAs, and (iv) detecting the antibody-bound hybrids.

In the methods according to the first, second, seventh, eighth, or thirteenth aspect of the present invention, it is preferred that the level of the miRNA is the expression level of said miRNA.

In the methods according to the first, second, seventh, eighth, or thirteenth aspect of the present invention, it is further preferred that the patient is a mammal, preferably a human.

In the methods according to the first, second, seventh, eighth, or thirteenth aspect of the present invention, it is also preferred that the blood sample is a whole blood sample or a blood fraction. It is more preferred that the blood fraction is serum, plasma, or a blood cell/cellular fraction. It is even more preferred that the blood fraction is a blood cell/cellular fraction. It is most preferred that the blood cell/cellular fraction comprises/essentially consists of/consists of erythrocytes, leukocytes, and/or thrombocytes, e.g. erythrocytes, leukocytes, and thrombocytes.

In the use according to the third, fourth, ninth, tenth, or fourteenth aspect of the present invention, it is preferred that the blood sample is a whole blood sample or a blood fraction. It is more preferred that the blood fraction is serum, plasma, or a blood cell/cellular fraction. It is even more preferred that the blood fraction is a blood cell/cellular fraction. It is most preferred that the blood cell/cellular fraction comprises/essentially consists of/consists of erythrocytes, leukocytes, and/or thrombocytes, e.g. erythrocytes, leukocytes, and thrombocytes.

In the kits according to the fifth, sixth, eleventh, twelfth, or fifteenth aspect of the present invention, it is preferred that the means are for determining the level (of each) of at least three miRNAs in a whole blood sample or a blood fraction. It is more preferred that the blood fraction is serum, plasma, or a blood cell/cellular fraction. It is even more preferred that the blood fraction is a blood cell/cellular fraction. It is most preferred that the blood cell/cellular fraction comprises/essentially consists of/consists of erythrocytes, leukocytes, and/or thrombocytes, e.g. erythrocytes, leukocytes, and thrombocytes.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURES

The following Figures are merely illustrative of the present invention and should not be construed to limit the scope of the invention as indicated by the appended claims in any way.

FIG. 1: Listing of miRNAs for determination of Alzheimer's Disease (AD) in a patient. Experimental details: SEQ ID NO: sequence identification number, miRNA: identifier of the miRNA according to miRBase, nucleotide sequence: sequence of the respective miRNA

FIG. 2: Performance of markers in the signatures. Listed are the current (V21) miRBase identification followed by the SEQ ID and the significance value in the first cohort (samples from US patients), the AUC in the first cohort, the p-value in the second cohort (samples from German patients) and the AUC in the second cohort. AUC values >0.5 are considered to represent up-regulated miRNAs, AUC values <0.5 are considered to represent down-regulated miRNAs. Two-tailed t-test p-values below the alpha level of 0.05 are considered significant.

FIG. 3: Performance of the signatures. Provided is the list of all miRNAs in the signatures, the SEQ ID of all miRNAs in the signatures, the signature complexity (number of miRNAs in the signature) and the worst case estimation of the performance. The results have been determined in a manner that the three performance values of specificity, sensitivity and accuracy are not significantly different (i.e. specificity=sensitivity=accuracy).

FIG. 4: Stem-loop reverse primer for reverse transcription. In order to determine the level of a miRNA referred to herein, the miRNA may be first transcribed into miRNA-specific complementary DNA (cDNA) using a stem-loop reverse primer having a nucleotide sequence selected from the group consisting of SEQ ID NO: 25 to SEQ ID NO: 35 by reverse transcriptase from total RNA. The miRNA-specific cDNA is then used as a template for real time polymerase chain reaction (RT-PCR), in particular real time quantitative polymerase chain reaction (RT-qPCR) (see FIG. 5 below). Experimental details: SEQ ID NO: sequence identification number of the respective miRNA, miRNA: identifier of the miRNA according to miRBase, SEQ ID NO: sequence identification number of the stem-loop reverse primer, nucleotide sequence of the stem-loop reverse primer.

FIG. 5: Forward primer for RT-PCR, in particular RT-qPCR. The produced miRNA-specific cDNA may then be used as a template for RT-PCR, in particular RT-qPCR. Suitable forward primers having a nucleotide sequence selected from the group consisting of SEQ ID NO: 36 to SEQ ID NO: 46 are shown. Experimental details: SEQ ID NO: sequence identification number of the miRNA, miRNA: identifier of the miRNA according to miRBase, SEQ ID NO: sequence identification number of the forward primer, nucleotide sequence of the forward primer.

FIG. 6: Reverse primer for RT-PCR, in particular RT-qPCR. The produced miRNA-specific cDNA may then be used as a template for RT-PCR, in particular RT-qPCR. A suitable universal reverse primer having a nucleotide sequence according to SEQ ID NO: 47 is shown. For example, for amplification of the miRNA having a nucleotide sequence according to SEQ ID NO: 1, the forward primer having a nucleotide sequence according to SEQ ID NO: 36 and the reverse primer having a nucleotide sequence according to SEQ ID NO: 47 may be used.

FIG. 7: Dual labelled probes are shown. A Dual-Labeled Probe is a single-stranded oligonucleotide labeled with two different dyes. A reporter dye is located at the 5′ end and a quencher molecule is located at the 3′ end. The quencher molecule inhibits the natural fluorescence emission of the reporter by fluorescence resonance energy transfer (FRET). The primer is elongated by the polymerase and the probe binds to the specific nucleotide template. Hydrolysis releases the reporter from the probe/target hybrid, causing an increase in fluorescence. The measured fluorescence signal is directly proportional to the amount of target miRNA. Experimental details: SEQ ID NO: sequence identification number of the miRNA, miRNA: identifier of the miRNA according to miRBase, SEQ ID NO: sequence identification number of the dual-labelled probe, Dual-labelled probe: sequence of the dual-labelled probe, with 56-FAM=5′ 6-FAM (Fluorescein), with 3IABLFQ=lowa black fluorescein quencher.

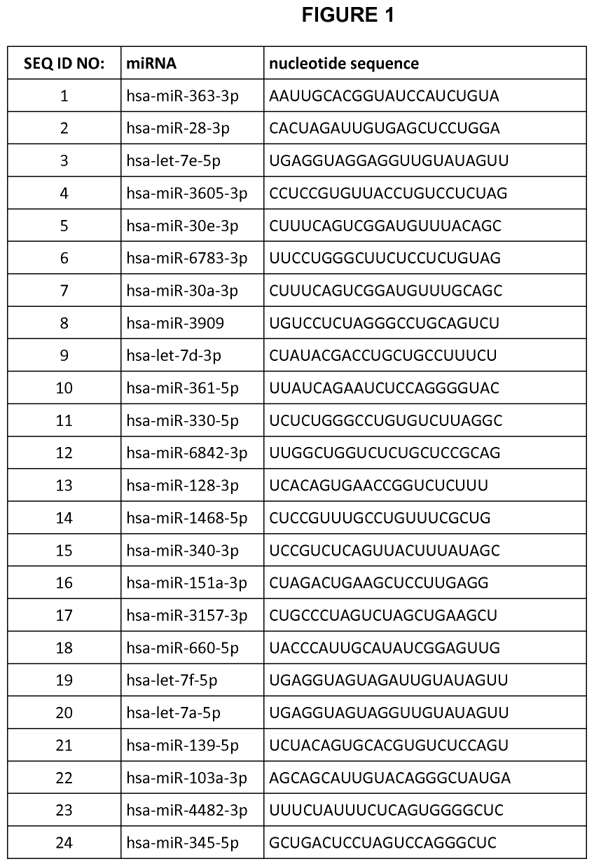

### EXAMPLES

The examples given below are for illustrative purposes only and do not limit the invention described above in any way.

### Example 1: US Cohort Analysis

### Patient Details

We analyzed the expression of miRNAs in peripheral blood of a total of 215 patients and healthy controls, either by NGS or by RT-qPCR or by both methods. In detail, we obtained 2.5 mL blood collected in PAXgene Blood RNA tubes (PreAnalytiX) from patients with AD (n=106) and from healthy controls (C) (n=22). Samples from patients with AD stem from the Biorepository and Tissue Bank PrecisionMed (San Diego, Calif., USA) (n=97) and the University Clinic of Erlangen (Germany) (n=9), samples from healthy controls stem from PrecisionMed (San Diego, Calif., USA). AD patients were diagnosed by using state of the art criteria. In detail, in order to be included in the probable AD group, patients fulfilled the following criteria of the NINCDS-ADRDA (National Institute of Neurological and Communicative Disorders and Stroke and the Alzheimer disease and Related Disorders Association): MISE >14 and <26, deficit in two or more areas of cognition, progressive worsening of memory and other cognitive functions, no disturbance of consciousness, onset between the ages of 40 and 90 years, most often after 65 years, and absence of systemic disorders or other brain diseases that could account for the progressive deterioration in cognition. Furthermore, MRI or CT reports that were compatible with AD are available. The median MISE score for the AD patients was 18.9 (3.4).

### RNA Isolation

The whole blood sample comprised in PAXgene Blood RNA tubes was centrifuged in order to separate the cellular fraction from the extracellular fraction (serum and plasma). The extracellular fraction was discarded. Total RNA including miRNA was isolated from blood cells using the PAXgene Blood miRNA Kit (Qiagen) following the manufacturer's recommendations. Isolated RNA was stored at −80° C. until use. RNA integrity was analyzed using Bioanalyzer 2100 (Agilent) and concentration and purity were measured using NanoDrop 2000 (Thermo Scientific).

### Library Preparation and Next-Generation Sequencing

We first analyzed samples from AD patients (n=48) and healthy controls (n=22) by Next Generation Sequencing (NGS). For the library preparation, 200 ng of total RNA was used per sample, as determined with a RNA 6000 Nano Chip on the Bioanalyzer 2100 (Agilent). Preparation was performed following the protocol of the TruSeq Small RNA Sample Prep Kit (Illumina). Concentration of the ready prepped libraries was measured on the Bioanalyzer using the DNA 1000 Chip. Libraries were then pooled in batches of six samples in equal amounts and clustered with a concentration of 9 pmol in one lane each of a single read flowcell using the cBot (Illumina). Sequencing of 50 cycles was performed on a HiSeq 2000 (Illumina). Demultiplexing of the raw sequencing data and generation of the fastq files was done using CASAVA v.1.8.2.

### NGS Data Analysis

The raw Illumina reads were first preprocessed by cutting the 3′ adapter sequence using the program fastx_clipper from the FASTX-Toolkit. Reads shorter than 18 nts after clipping were removed. The remaining reads are reduced to unique reads and their frequency per sample to make the mapping steps more time efficient. For the remaining steps, we used the miRDeep2 pipeline (Friedländer M R, Mackowiak S D, Li N, Chen W, Rajewsky N: miRDeep2 accurately identifies known and hundreds of novel microRNA genes in seven animal clades. Nucleic Acids Res 2012, 40:37-52). These steps consist of mapping the reads against the genome (hg19), mapping the reads against miRNA precursor sequences from miRBase release v18, summarizing the counts for the samples, and the prediction of novel miRNAs. Since the miRDeep2 pipeline predicts in our case the novel miRNAs per sample, we merged the miRNAs afterwards as follows: first, we extract the novel miRNAs per sample that have a signal-to-noise ratio >10. Subsequently, we merge only those novel miRNAs that are located on the same chromosome, and both their mature forms share an overlap of at least 11 nucleotides. The remaining putative novel miRNAs were mapped with BLAST (v 2.2.24, Altschul S F, Gish W, Miller W, Myers E W, Lipman D J: Basic local alignment search tool. J Molecular Biol 1990, 215:403-410) against known ncRNA and miRNA sequences from diverse sources (miRBase v18, Kozomara A, Griffiths-Jones S: miRBase: integrating microRNA annotation and deep-sequencing data. Nucleic Acids Res 2011, 39:D152-15), snoRNA-LBME-db (Lestrade L, Weber M J: snoRNA-LBME-db, a comprehensive database of human H/ACA and C/D box snoRNAs. Nucleic Acids Res 2006, 34:D158-162), ncRNAs from Ensembl ‘Homo_sapiens.GRCh37.67. ncrna.fa’, NONCODE v3.0 (Bu D, Yu K, Sun S, Xie C, Skogerbo G, Miao R, Xiao H, Liao Q, Luo H, Zhao G, Zhao H, Liu Z, Liu C, Chen R, Zhao Y: NONCODE v3.0: integrative annotation of long noncoding RNAs. Nucleic Acids Res 2012, 40:D210-215). We excluded sequences that aligned with >90% of their length (allowing 1 mismatch) to any of the ncRNA sequences. All NGS data are publicly available in GEO database (GSE46579).

### Bioinformatics Analysis

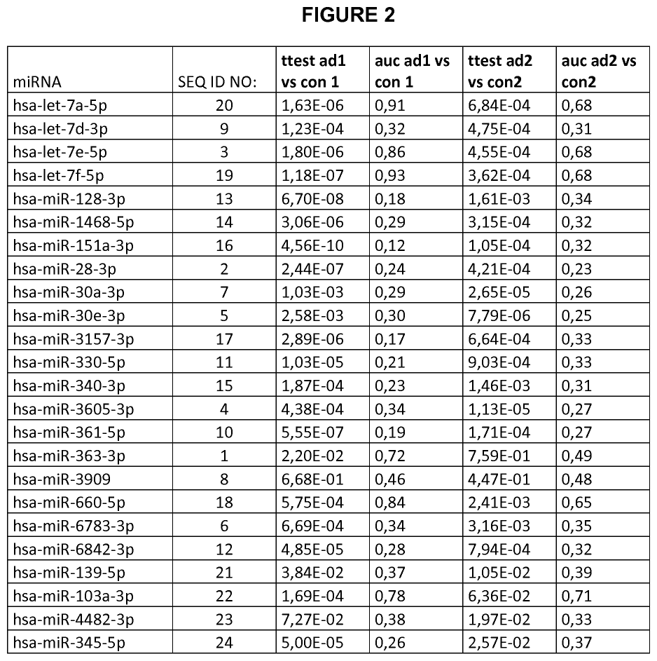

For the NGS analysis, we excluded miRNAs with <50 read counts summed up across all samples of each group (AD or control), since these were considered lowly abundant. We normalized the read counts using standard quantile normalization. Next, we calculated for each miRNA the area under the receiver operator characteristic curve (AUC), the fold-change, and the significance value (P value) using Wilcoxon-Mann-Whitney (WMW) test. All significance values were adjusted for multiple testing using the Benjamini-Hochberg approach (Benjamini Y, Drai D, Elmer G, Kafkafi N, Golani I: Controlling the false discovery rate in behavior genetics research. Behav Brain Res 2001, 125:279-284; Hochberg Y: A sharper bonferroni procedure for multiple tests of significance. Biometrica 1988, 75:185-193). The bioinformatics analyses have been carried out using the freely available tool R (Team R: R: A Languageand Environment for Statistical Computing. Vienna: R Foundation for Statistical Computing 2008). For classification purposes, we used support vector machines (SVM) from the R package e1071. If not stated otherwise, we computed the group-wise classifications using linear kernels in 10-fold cross-validations with 100 repetitions. In addition, we computed the classification of permuted class labels with the same parameters as control. If group sizes were unbalanced, we randomly selected samples from the bigger group to match the sample sizes in the smaller group in each repetition. The results are shown in FIG. 2 (ttest ad1 vs con1, auc ad1 vs con1).

### Example 2: German Cohort Analysis

Patients and miRNA Profiling

We collected 2.5-mL blood from AD patients (n=49) and healthy controls (n=55) in PAXgene Blood RNA (PreAnalytiX) tubes. The analytical procedure was performed as follows: In brief, we centrifuged the whole blood sample comprised in PAXgene Blood RNA tubes in order to separate the cellular fraction from the extracellular fraction (serum and plasma). We discarded the extracellular fraction. From the tubes, total RNA was isolated from blood cells using the PAXgene Blood miRNA Kit (Qiagen) following the manufacturer's instruction. For sequencing library preparation, 200 ng of total RNA was used (quantified by RNA 6000 Nano Chip using Bioanalyzer 2100 [Agilent]). Preparation was performed according to the protocol of the TruSeq Small RNA Sample Prep Kit (IIlumina). Concentration of the ready prepped libraries was measured by using the Bioanalyzer (DNA 1000 Chip). Libraries were then clustered with a concentration of 9 pmol with six samples in one lane. Sequencing of 50 cycles was performed on a HiSeq 2000 instrument (Illumina) and demultiplexing of the raw sequencing data was done using CASAVA version 1.8.2.

### Statistical Analysis

All 290 samples were processed by miRDeep2 as described above before downstream analysis in R (version 3.0.2) had been carried out. For all samples together, quantile normalization was performed and all miRNAs with, 5 reads in less than five samples were excluded to minimize noise. This procedure resulted in a set of 580 miRNAs that were further investigated. Where applicable, P values were adjusted for multiple testing using Benjamini-Hochberg correction. For hypothesis testing, we calculated unpaired two-tailed t tests. Because not all miRNAs were normally distributed, we also calculated nonparametric Wilcoxon Mann-Whitney (WMW) tests (unpaired, two tailed). Beyond the hypothesis tests, the area under the receiver operator characteristic curves (AUC) was calculated for each miRNA. For correlating AUCs in both cohorts, AUCs were provided in an interval between 0 and 1. miRNAs with higher expression in AD have AUC, 0.5 and miRNAs with higher expression in controls 0.0.5, miRNAs that are equally abundant have AUCs of around 0.5. To calculate confidence intervals (CIs) for the AUC, 1000 bootstrap samples have been performed using the pROC package. As further statistical approaches, we performed hierarchical clustering as implemented in the Heatplus R package (read counts were transformed to z-scores and complete linkage clustering relying on the Euclidian distance was done). We also carried out principal component analysis (PCA) as implemented in the prcomp R package and showed the first versus second principal component as scatter plot. Finally, analysis of variance (ANOVA) has been applied to the two groups: AD and unaffected controls.

To combine the predictive power of multiple miRNAs, machine learning has been performed. In detail, support vector machines using a radial basis function as kernel were trained and evaluated using fivefold cross validation on the complete data set. To account for variations between different cross-validation runs, the procedure has been repeated with 20 random partitions in test and training data. To select most informative miRNAs with respect to AD, a stepwise forward feature selection based on the P values has been carried out. Here, in each iteration, the k features (k was varied between two and 500 features) with lowest P values in the training part of the cross validation were selected and subsequently evaluated on the test sample part. To check for potential over training, 20 repetitions of permutation tests have been performed. Here, the complete subset selection step as well as the classification was carried out with randomly permuted class labels. The results are shown in FIG. 2 (ttest ad2 vs con2, auc ad2 vs con2).

### Example 3: Combinatory Analysis of US and German Cohort

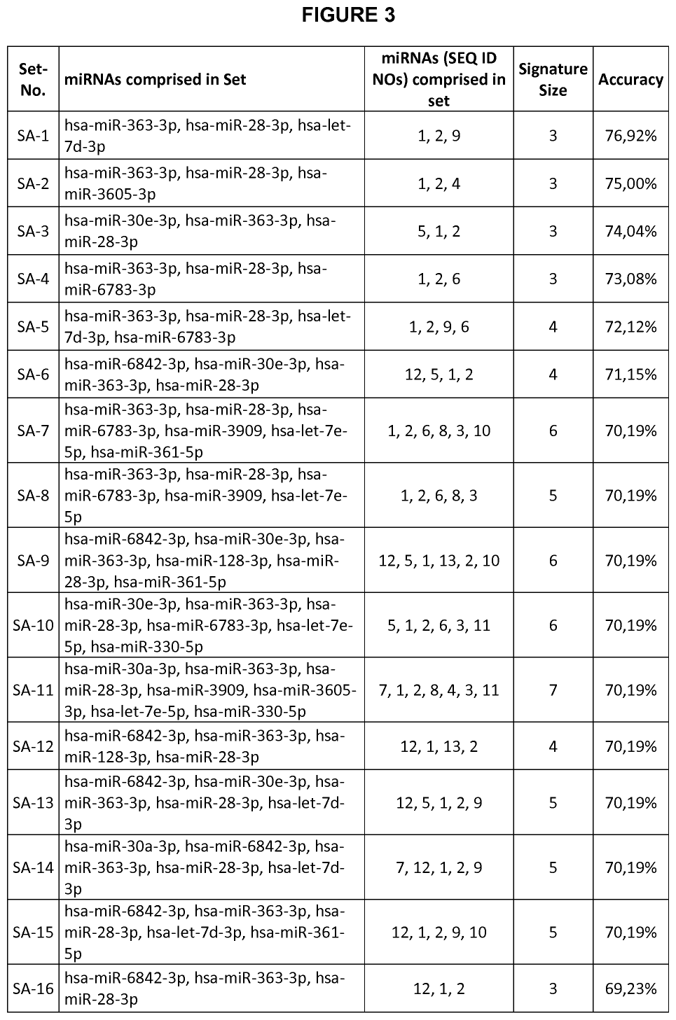

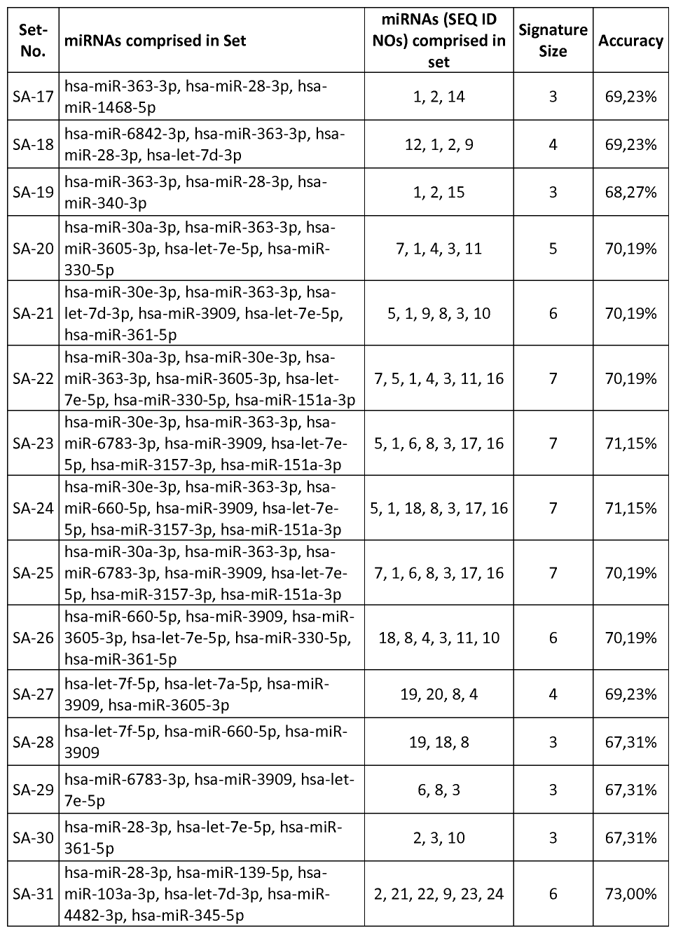

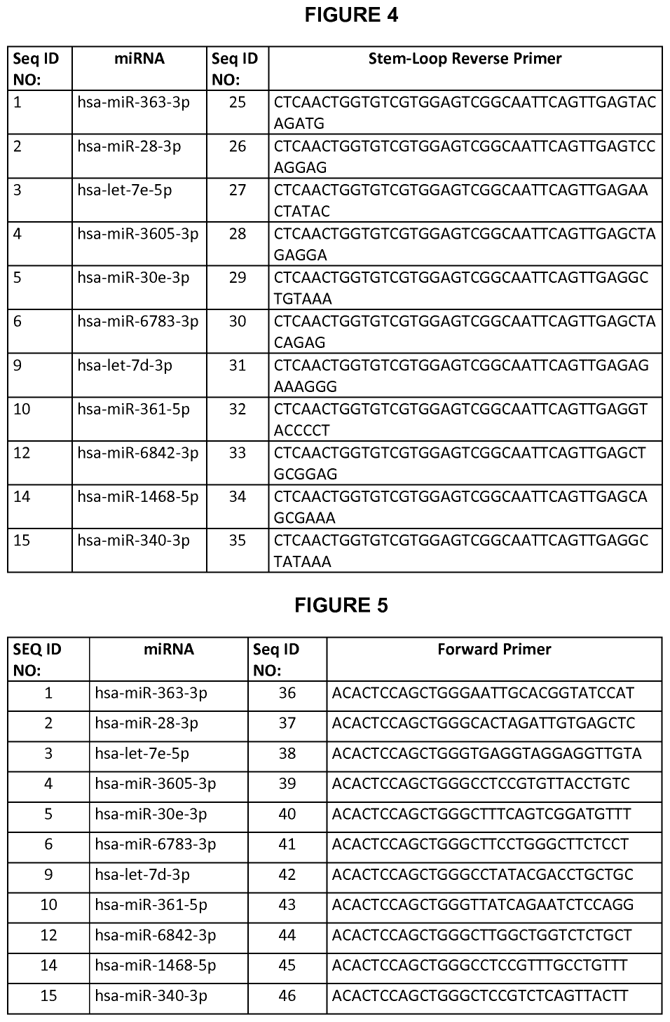

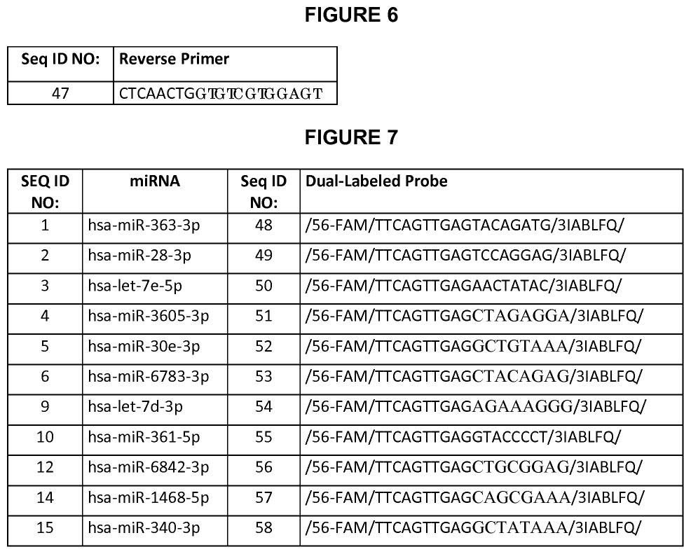

In the present invention signatures derived from two cohorts of Alzheimer's Disease (AD) patients and unaffected individuals (UI) were developed. For AD samples and UI samples, whole blood miRNA profiles were generated. The AD samples and UIs were sequenced and pre-processed as described above (US Cohort: Example 1; German Cohort: Example 2). Aim of the study was to compute signatures with an adequate complexity. To this end, not only the cross-validation (re-sampling) error has been calculated but also the blind test error where models based on signatures obtained in the United States have been applied to predict the German cohort. Since a naive brute-fore implementation that considers all potential signatures would correspond to an overtraining and would anyhow not been feasible (for 816 miRNAs over 18 Billion 4-miRNA signatures exist) a straightforward genetic algorithm has been applied. The algorithm was allowed to run maximal 10,000 iterations or to stop after convergence. As classification approach radial basis function support vector machines with class weights have been used. For each signature the re-sampling error and the blind test performance has been computed. For the best signatures consisting of 3 to 7 markers, the respective performance rates are presented in FIG. 3. The cross-validation and blind test error showed a reasonable correlation of 0.55 but at the same time highlighted that the signatures with maximal performance in terms of re-sampling accuracy not necessarily were best in the blind test accuracy. The best signature in this regard consisting of hsa-let-7a-5p, hsa-miR-6783-3p and hsa-miR-151a-3p had a resampling accuracy of 94.7% while the blind test accuracy was 57.7% only. The best accuracy resulting from the genetic algorithm had a resampling accuracy of 81.6% and a blind test accuracy of 76.9% (see SA-1).

Our new signatures are, thus, the first stable Alzheimer signatures that hold in different countries and for groups with different ethnical background.

1. A method for determining Alzheimer's Disease in a patient comprising the steps of:
(i) determining the level (of each) of at least 3 miRNAs comprised in a set in a blood sample isolated from a patient, wherein the first miRNA has a nucleotide sequence according to SEQ ID NO: 1 (hsa-miR-363-3p), and
(ii) comparing the level (of each) of the at least 3 miRNAs comprised in the set with a reference level of said at least three miRNAs, wherein the comparison allows to determine Alzheimer's Disease in the patient.

2. The method of claim 1, wherein
a) the second and third miRNAs have nucleotide sequences selected from the group consisting of SEQ ID NO: 2 to SEQ ID NO: 18,
(b) the second miRNA has a nucleotide sequence according to SEQ ID NO: 2 (hsa-miR-28-3p) or SEQ ID NO: 3 (hsa-let-7e-5p) and the third miRNA has a nucleotide sequence selected from the group consisting of SEQ ID NO: 3 to SEQ ID NO: 18 under the proviso that the second and third miRNAs are different,
(c) the second miRNA has nucleotide sequence according to SEQ ID NO: 2 and the third miRNA has a nucleotide sequence selected from the group consisting of SEQ ID NO: 3 to SEQ ID NO: 15,
(d) the second miRNA has a nucleotide sequence according to SEQ ID NO: 3 and the third miRNA has a nucleotide sequence selected from the group consisting of SEQ ID NO: 2, SEQ ID NO: 4 to SEQ ID NO: 11, and SEQ ID NO: 16 to SEQ ID NO: 18, or
(e) the second miRNA has a nucleotide sequence according to SEQ ID NO: 2 (hsa-miR-28-3p) and the third miRNA has a nucleotide sequence according to SEQ ID NO: 3 (hsa-let-7e-5p).

3-6. (canceled)

7. The method of claim 1, wherein the at least three miRNAs are comprised in a set selected from the group consisting of:
(a) SEQ ID NO: 1, SEQ ID NO: 2, and SEQ ID NO: 9,
(b) SEQ ID NO: 1, SEQ ID NO: 2, and SEQ ID NO: 4,
(c) SEQ ID NO: 1, SEQ ID NO: 2, and SEQ ID NO: 5,
(d) SEQ ID NO: 1, SEQ ID NO: 2, and SEQ ID NO: 6,
(e) SEQ ID NO: 1, SEQ ID NO: 2, SEQ ID NO: 6, and SEQ ID NO: 9,
(f) SEQ ID NO: 1, SEQ ID NO: 2, SEQ ID NO: 5, and SEQ ID NO: 12,
(g) SEQ ID NO: 1, SEQ ID NO: 2, SEQ ID NO: 3, SEQ ID NO: 6, SEQ ID NO: 8, and SEQ ID NO: 10,
(h) SEQ ID NO: 1, SEQ ID NO: 2, SEQ ID NO: 3, SEQ ID NO: 6, and SEQ ID NO: 8,
(i) SEQ ID NO: 1, SEQ ID NO: 2, SEQ ID NO: 5, SEQ ID NO: 10, SEQ ID NO: 12, and SEQ ID NO: 13,
(j) SEQ ID NO: 1, SEQ ID NO: 2, SEQ ID NO: 3, SEQ ID NO: 5, SEQ ID NO: 6, and SEQ ID NO: 11,
(k) SEQ ID NO: 1, SEQ ID NO: 2, SEQ ID NO: 3, SEQ ID NO: 4, SEQ ID NO: 7, SEQ ID NO: 8, and SEQ ID NO: 11,
(l) SEQ ID NO: 1, SEQ ID NO: 2, SEQ ID NO: 12, and SEQ ID NO: 13,
(m) SEQ ID NO: 1, SEQ ID NO: 2, SEQ ID NO: 5, SEQ ID NO: 9, and SEQ ID NO: 12,
(n) SEQ ID NO: 1, SEQ ID NO: 2, SEQ ID NO: 7, SEQ ID NO: 9, and SEQ ID NO: 12,
(o) SEQ ID NO: 1, SEQ ID NO: 2, SEQ ID NO: 9, SEQ ID NO: 10, and SEQ ID NO: 12,
(p) SEQ ID NO: 1, SEQ ID NO: 2, and SEQ ID NO: 12,
(q) SEQ ID NO: 1, SEQ ID NO: 2, and SEQ ID NO: 14,
(r) SEQ ID NO: 1, SEQ ID NO: 2, SEQ ID NO: 9, and SEQ ID NO: 12,
(s) SEQ ID NO: 1, SEQ ID NO: 2, and SEQ ID NO: 15,
(t) SEQ ID NO: 1, SEQ ID NO: 3, SEQ ID NO: 4, SEQ ID NO: 7, and SEQ ID NO: 11,
(u) SEQ ID NO: 1, SEQ ID NO: 3, SEQ ID NO: 5, SEQ ID NO: 8, SEQ ID NO: 9, and SEQ ID NO: 10,
(v) SEQ ID NO: 1, SEQ ID NO: 3, SEQ ID NO: 4, SEQ ID NO: 5, SEQ ID NO: 7, SEQ ID NO: 11, and SEQ ID NO: 16,
(w) SEQ ID NO: 1, SEQ ID NO: 3, SEQ ID NO: 5, SEQ ID NO: 6, SEQ ID NO: 8, SEQ ID NO: 16, and SEQ ID NO: 17,
(x) SEQ ID NO: 1, SEQ ID NO: 3, SEQ ID NO: 5, SEQ ID NO: 8, SEQ ID NO: 16, SEQ ID NO: 17, and SEQ ID NO: 18, and
(y) SEQ ID NO: 1, SEQ ID NO: 3, SEQ ID NO: 6, SEQ ID NO: 7, SEQ ID NO: 8, SEQ ID NO: 16, and SEQ ID NO: 17.

8. The method of claim 1, wherein the reference level is the level determined by measuring at least one reference blood sample from
at least one healthy subject,
wherein the determination of Alzheimer's Disease comprises diagnosing whether the patient suffers from Alzheimer's Disease, and
wherein the level of the miRNA having a nucleotide sequence selected from the group consisting of SEQ ID NO: 1, SEQ ID NO: 3, and SEQ ID NO: 18 above the reference level determined by measuring at least one reference blood sample from at least one healthy subject indicates that the patient suffers from Alzheimer's Disease, and/or
the level of the miRNA having a nucleotide sequence selected from the group consisting of SEQ ID NO: 2 and SEQ ID NO: 4 to SEQ ID NO: 17 below the reference level determined by measuring at least one reference blood sample from at least one healthy subject indicates that the patient suffers from Alzheimer's Disease.

9-10. (canceled)

11. The method of claim 1,
wherein the reference level is the level determined by measuring at least one reference blood sample from at least one healthy subject,
wherein the determination of Alzheimer's Disease comprises determining whether Alzheimer's Disease is present in the patient, and
wherein the level of the miRNA having a nucleotide sequence selected from the group consisting of SEQ ID NO: 1, SEQ ID NO: 3, and SEQ ID NO: 18 above the reference level determined by measuring at least one reference blood sample from at least one healthy subject indicates that Alzheimer's Disease is present in the patient, and/or
the level of the miRNA having a nucleotide sequence selected from the group consisting of SEQ ID NO: 2 and SEQ ID NO: 4 to SEQ ID NO: 17 below the reference level determined by measuring at least one reference blood sample from at least one healthy subject indicates that Alzheimer's Disease is present in the patient.

12. (canceled)

13. The method of claim 1,
wherein the reference level is the level determined by measuring at least one reference blood sample from at least one healthy subject,
wherein the determination of Alzheimer's Disease comprises determining whether Alzheimer's Disease is absent in the patient, and
wherein the level of the miRNA having a nucleotide sequence selected from the group consisting of SEQ ID NO: 1, SEQ ID NO: 3, and SEQ ID NO: 18 comparable with the reference level determined by measuring at least one reference blood sample from at least one healthy subject indicates that Alzheimer's Disease is absent in the patient,
the level of the miRNA having a nucleotide sequence selected from the group consisting of SEQ ID NO: 2 and SEQ ID NO: 4 to SEQ ID NO: 17 comparable with the reference level determined by measuring at least one reference blood sample from at least one healthy subject indicates that Alzheimer's Disease is absent in the patient,
the level of the miRNA having a nucleotide sequence selected from the group consisting of SEQ ID NO: 1, SEQ ID NO: 3, and SEQ ID NO: 18 below the reference level determined by measuring at least one reference blood sample from at least one subject having Alzheimer's Disease indicates that Alzheimer's Disease is absent in the patient, and/or
the level of the miRNA having a nucleotide sequence selected from the group consisting of SEQ ID NO: 2 and SEQ ID NO: 4 to SEQ ID NO: 17 above the reference level determined by measuring at least one reference blood sample from at least one subject having Alzheimer's Disease indicates that Alzheimer's Disease is absent in the patient.

14. (canceled)

15. The method of claim 1,
wherein the reference level is the level determined by measuring at least one reference blood sample from at least one healthy subject,
wherein the determination of Alzheimer's Disease comprises determining whether the patient is at risk for developing Alzheimer's Disease, and
wherein the level of the miRNA having a nucleotide sequence selected from the group consisting of SEQ ID NO: 1, SEQ ID NO: 3, and SEQ ID NO: 18 above the reference level determined by measuring at least one reference blood sample from at least one healthy subject indicates that the patient is at risk for developing Alzheimer's Disease, and/or
the level of the miRNA having a nucleotide sequence selected from the group consisting of SEQ ID NO: 2 and SEQ ID NO: 4 to SEQ ID NO: 17 below the reference level determined by measuring at least one reference blood sample from at least one healthy subject indicates that the patient is at risk for developing Alzheimer's Disease.

16. (canceled)

17. The method of claim 1,
wherein the determination comprises determining the course of Alzheimer's Disease in the patient,
wherein said determining comprises determining the level (of each) of the at least three miRNAs comprised in the set in the blood sample at a first point in time and in at least one further blood sample at a later point in time and comparing said levels determined at the different time points, and
wherein the level of the miRNA having a nucleotide sequence selected from the group consisting SEQ ID NO: 1, SEQ ID NO: 3, and SEQ ID NO: 18 which
(i) increases over time indicates that Alzheimer's Disease worsens in the patient,
(ii) does not change over time indicates that Alzheimer's Disease does not worsen/is stable in the patient, or
(iii) decreases over time indicates that Alzheimer's Disease improves in the patient, and/or
the level of the miRNA having a nucleotide sequence selected from the group consisting of SEQ ID NO: 2 and SEQ ID NO: 4 to SEQ ID NO: 17 which
(i) decreases over time indicates that Alzheimer's Disease worsens in the patient,
(ii) does not change over time indicates that Alzheimer's Disease does not worsen/is stable in the patient, or
(iii) increases over time indicates that Alzheimer's Disease improves in the patient.

18-19. (canceled)

20. The method of claim 17, wherein the patient receives or has received a treatment of Alzheimer's Disease.

21. The method of claim 20, wherein the treatment of Alzheimer's Disease is selected from the group consisting of the administration of a drug, cognitive training, ergotherapy, and psychotherapy.

22. The method of claim 1, wherein the blood sample is selected from the group consisting of whole blood and a blood cellular fraction.

23-24. (canceled)

25. A method for determining Alzheimer's Disease in a patient comprising the steps of:
(i) determining the level (of each) of at least 3 miRNAs in a blood sample isolated from a patient, wherein the at least three miRNAs are comprised in a set selected from the group consisting of:
(a) SEQ ID NO: 1, SEQ ID NO: 2, and SEQ ID NO: 9,
(b) SEQ ID NO: 1, SEQ ID NO: 2, and SEQ ID NO: 4,
(c) SEQ ID NO: 1, SEQ ID NO: 2, and SEQ ID NO: 5,
(d) SEQ ID NO: 1, SEQ ID NO: 2, and SEQ ID NO: 6,
(e) SEQ ID NO: 1, SEQ ID NO: 2, SEQ ID NO: 6, and SEQ ID NO: 9,
(f) SEQ ID NO: 1, SEQ ID NO: 2, SEQ ID NO: 5, and SEQ ID NO: 12,
(g) SEQ ID NO: 1, SEQ ID NO: 2, SEQ ID NO: 3, SEQ ID NO: 6, SEQ ID NO: 8, and SEQ ID NO: 10,
(h) SEQ ID NO: 1, SEQ ID NO: 2, SEQ ID NO: 3, SEQ ID NO: 6, and SEQ ID NO: 8,
(i) SEQ ID NO: 1, SEQ ID NO: 2, SEQ ID NO: 5, SEQ ID NO: 10, SEQ ID NO: 12, and SEQ ID NO: 13,
(j) SEQ ID NO: 1, SEQ ID NO: 2, SEQ ID NO: 3, SEQ ID NO: 5, SEQ ID NO: 6, and SEQ ID NO: 11,
(k) SEQ ID NO: 1, SEQ ID NO: 2, SEQ ID NO: 3, SEQ ID NO: 4, SEQ ID NO: 7, SEQ ID NO: 8, and SEQ ID NO: 11,
(l) SEQ ID NO: 1, SEQ ID NO: 2, SEQ ID NO: 12, and SEQ ID NO: 13,
(m) SEQ ID NO: 1, SEQ ID NO: 2, SEQ ID NO: 5, SEQ ID NO: 9, and SEQ ID NO: 12,
(n) SEQ ID NO: 1, SEQ ID NO: 2, SEQ ID NO: 7, SEQ ID NO: 9, and SEQ ID NO: 12,
(o) SEQ ID NO: 1, SEQ ID NO: 2, SEQ ID NO: 9, SEQ ID NO: 10, and SEQ ID NO: 12,
(p) SEQ ID NO: 1, SEQ ID NO: 2, and SEQ ID NO: 12,
(q) SEQ ID NO: 1, SEQ ID NO: 2, and SEQ ID NO: 14,
(r) SEQ ID NO: 1, SEQ ID NO: 2, SEQ ID NO: 9, and SEQ ID NO: 12,
(s) SEQ ID NO: 1, SEQ ID NO: 2, and SEQ ID NO: 15,
(t) SEQ ID NO: 1, SEQ ID NO: 3, SEQ ID NO: 4, SEQ ID NO: 7, and SEQ ID NO: 11,
(u) SEQ ID NO: 1, SEQ ID NO: 3, SEQ ID NO: 5, SEQ ID NO: 8, SEQ ID NO: 9, and SEQ ID NO: 10,
(v) SEQ ID NO: 1, SEQ ID NO: 3, SEQ ID NO: 4, SEQ ID NO: 5, SEQ ID NO: 7, SEQ ID NO: 11, and SEQ ID NO: 16,
(w) SEQ ID NO: 1, SEQ ID NO: 3, SEQ ID NO: 5, SEQ ID NO: 6, SEQ ID NO: 8, SEQ ID NO: 16, and SEQ ID NO: 17,
(x) SEQ ID NO: 1, SEQ ID NO: 3, SEQ ID NO: 5, SEQ ID NO: 8, SEQ ID NO: 16, SEQ ID NO: 17, and SEQ ID NO: 18, and
(y) SEQ ID NO: 1, SEQ ID NO: 3, SEQ ID NO: 6, SEQ ID NO: 7, SEQ ID NO: 8, SEQ ID NO: 16, and SEQ ID NO: 17,
and
(ii) comparing the level (of each) of the at least 3 miRNAs comprised in the set with a reference level of said at least three miRNAs, wherein the comparison allows to determine Alzheimer's Disease in the patient.

26-36. (canceled)

37. A kit for determining Alzheimer's Disease in a patient comprising:
(i) means for determining the level (of each) of at least three miRNAs comprised in a set in a blood sample isolated from a patient,
wherein the first miRNA has a nucleotide sequence according to SEQ ID NO: 1 (hsa-miR-363-3p), and
(ii) optionally at least three references.

38. The kit of claim 37, wherein
(a) the second and third miRNAs have nucleotide sequences selected from the group consisting of SEQ ID NO: 2 to SEQ ID NO: 18,
(b) the second miRNA has a nucleotide sequence according to SEQ ID NO: 2 (hsa-miR-28-3p) or SEQ ID NO: 3 (hsa-let-7e-5p) and the third miRNA has a nucleotide sequence selected from the group consisting of SEQ ID NO: 3 to SEQ ID NO: 18 under the proviso that the second and third miRNAs are different,
(c) the second miRNA has nucleotide sequence according to SEQ ID NO: 2 and the third miRNA has a nucleotide sequence selected from the group consisting of SEQ ID NO: 3 to SEQ ID NO: 15,
(d) the second miRNA has a nucleotide sequence according to SEQ ID NO: 3 and the third miRNA has a nucleotide sequence selected from the group consisting of SEQ ID NO: 2, SEQ ID NO: 4 to SEQ ID NO: 11, and SEQ ID NO: 16 to SEQ ID NO: 18, or
(e) the second miRNA has a nucleotide sequence according to SEQ ID NO: 2 (hsa-miR-28-3p) and the third miRNA has a nucleotide sequence according to SEQ ID NO: 3 (hsa-let-7e-5p).

39-42. (canceled)

43. The kit of claim 37, wherein the at least three miRNAs are comprised in a set selected from the group consisting of:
(a) SEQ ID NO: 1, SEQ ID NO: 2, and SEQ ID NO: 9,
(b) SEQ ID NO: 1, SEQ ID NO: 2, and SEQ ID NO: 4,
(c) SEQ ID NO: 1, SEQ ID NO: 2, and SEQ ID NO: 5,
(d) SEQ ID NO: 1, SEQ ID NO: 2, and SEQ ID NO: 6,
(e) SEQ ID NO: 1, SEQ ID NO: 2, SEQ ID NO: 6, and SEQ ID NO: 9,
(f) SEQ ID NO: 1, SEQ ID NO: 2, SEQ ID NO: 5, and SEQ ID NO: 12,
(g) SEQ ID NO: 1, SEQ ID NO: 2, SEQ ID NO: 3, SEQ ID NO: 6, SEQ ID NO: 8, and SEQ ID NO: 10,
(h) SEQ ID NO: 1, SEQ ID NO: 2, SEQ ID NO: 3, SEQ ID NO: 6, and SEQ ID NO: 8,
(i) SEQ ID NO: 1, SEQ ID NO: 2, SEQ ID NO: 5, SEQ ID NO: 10, SEQ ID NO: 12, and SEQ ID NO: 13,
(j) SEQ ID NO: 1, SEQ ID NO: 2, SEQ ID NO: 3, SEQ ID NO: 5, SEQ ID NO: 6, and SEQ ID NO: 11,
(k) SEQ ID NO: 1, SEQ ID NO: 2, SEQ ID NO: 3, SEQ ID NO: 4, SEQ ID NO: 7, SEQ ID NO: 8, and SEQ ID NO: 11,
(l) SEQ ID NO: 1, SEQ ID NO: 2, SEQ ID NO: 12, and SEQ ID NO: 13,
(m) SEQ ID NO: 1, SEQ ID NO: 2, SEQ ID NO: 5, SEQ ID NO: 9, and SEQ ID NO: 12,
(n) SEQ ID NO: 1, SEQ ID NO: 2, SEQ ID NO: 7, SEQ ID NO: 9, and SEQ ID NO: 12,
(o) SEQ ID NO: 1, SEQ ID NO: 2, SEQ ID NO: 9, SEQ ID NO: 10, and SEQ ID NO: 12,
(p) SEQ ID NO: 1, SEQ ID NO: 2, and SEQ ID NO: 12,
(q) SEQ ID NO: 1, SEQ ID NO: 2, and SEQ ID NO: 14,
(r) SEQ ID NO: 1, SEQ ID NO: 2, SEQ ID NO: 9, and SEQ ID NO: 12,
(s) SEQ ID NO: 1, SEQ ID NO: 2, and SEQ ID NO: 15,
(t) SEQ ID NO: 1, SEQ ID NO: 3, SEQ ID NO: 4, SEQ ID NO: 7, and SEQ ID NO: 11,
(u) SEQ ID NO: 1, SEQ ID NO: 3, SEQ ID NO: 5, SEQ ID NO: 8, SEQ ID NO: 9, and SEQ ID NO: 10,
(v) SEQ ID NO: 1, SEQ ID NO: 3, SEQ ID NO: 4, SEQ ID NO: 5, SEQ ID NO: 7, SEQ ID NO: 11, and SEQ ID NO: 16,
(w) SEQ ID NO: 1, SEQ ID NO: 3, SEQ ID NO: 5, SEQ ID NO: 6, SEQ ID NO: 8, SEQ ID NO: 16, and SEQ ID NO: 17,
(x) SEQ ID NO: 1, SEQ ID NO: 3, SEQ ID NO: 5, SEQ ID NO: 8, SEQ ID NO: 16, SEQ ID NO: 17, and SEQ ID NO: 18, and
(y) SEQ ID NO: 1, SEQ ID NO: 3, SEQ ID NO: 6, SEQ ID NO: 7, SEQ ID NO: 8, SEQ ID NO: 16, and SEQ ID NO: 17.

44. The kit of claim 37, wherein the determination of Alzheimer's Disease comprises diagnosing whether the patient suffers from Alzheimer's Disease, determining whether Alzheimer's Disease is present in the patient, determining whether Alzheimer's Disease is absent in the patient, and/or determining the course of Alzheimer's Disease in the patient.

45. The kit of claim 37, wherein the means for determining the level (of each) of the at least three miRNA in the blood sample from the patient comprise at least three polynucleotides for detecting miRNAs.

46. (canceled)

47. The kit of claim 37, wherein the kit further comprises
(iii) a container, and/or
(iv) a data carrier.

48. The kit of claim 47, wherein the data carrier comprises instructions on how to use the kit.

49. (canceled)
